(19) United States
(12) Patent Application Publication
(10) Pub. No.: US 2026/0022352 A1
(43) Pub. Date: Jan. 22, 2026

(54) COMPOSITIONS AND METHODS FOR PURIFYING LENTIVIRUS PARTICLES

(71) Applicant: North Carolina State University, Raleigh, NC (US)

(72) Inventors: Stefano Menegatti, Raleigh, NC (US); Eduardo Barbieri, Raleigh, NC (US)

(21) Appl. No.: 19/348,346

(22) Filed: Oct. 2, 2025

Related U.S. Application Data
(63) Continuation of application No. PCT/US2024/041320, filed on Aug. 7, 2024.
(60) Provisional application No. 63/518,035, filed on Aug. 7, 2023.

(51) Int. Cl. C12N 7/02; B01J 20/26; C07K 17/00

(52) U.S. Cl. CPC C12N 7/025; B01J 20/262; C07K 17/00; C12N 2740/15052

(57) ABSTRACT

The present disclosure provides materials and methods related to the purification of viral vectors. In particular, the present disclosure provides peptides, compositions, adsorbents, and related methods, capable of removing process-related impurities (e.g., host cell proteins, nucleic acids, and media components) and product-related impurities (e.g., product fragments, product aggregates, and inactive forms derived from product degradation by or association with other species in the cell culture harvest) from samples during the production and purification of lentivirus particles.

### CROSS REFERENCE TO RELATED APPLICATIONS

This application is a continuation of PCT International Application No. PCT/US2024/041320, filed Aug. 7, 2024, which claims priority to and the benefit of U.S. Provisional Patent Application No. 63/518,035, filed Aug. 7, 2023, both of which are incorporated herein by reference in their entireties for all purposes.

### INCORPORATION-BY-REFERENCE OF MATERIAL SUBMITTED ELECTRONICALLY

Incorporation by reference in its entirety herein is a computer-readable nucleotide/amino acid sequence listing submitted concurrently herewith and identified as follows: One 100,535 Byte file named “NCSU_42204_601_SequenceListing.xml” created on Aug. 5, 2024.

### FIELD

The present disclosure provides materials and methods related to the purification of viral vectors. In particular, the present disclosure provides peptide ligands, compositions, adsorbents, and related methods, capable of removing process-related impurities and product-related impurities from samples during the production and purification of lentivirus.

### BACKGROUND

The recent swath of FDA approvals of ex vivo cell therapies, ABECMA™ and CARVYKTI™ for multiple myeloma, BREYANZI™ for B-cell lymphoma, SKYSONA™ for cerebral adrenoleukodystrophy, and ZYNTEGLO™ for 0-thalassemia, has turned the spotlight on lentiviruses as a replication-defective viral vector for gene and cell therapy. Lentiviral vectors (LVVs) comprise a single strand RNA of 8.5 to 9 kb in length, packed inside a capsid and enveloped by a coat with 80-100 nm diameter. The envelope displays many copies of vesicular stomatitis virus glycoproteins G (VSV-G), which influence virus stability and cellular tropism. The VSV-G protein interacts with the low-density lipoprotein receptor (LDL-R) on the membrane of the target cell and mediates virus infection by membrane fusion.

Despite their clinical relevance, LVVs do not yet benefit from an established biomanufacturing platform. In particular, the downstream segment of bioprocessing represents a critical bottleneck in the industry, causing high waiting times and cost to patients (up to $2.8 million per treatment). LVVs are traditionally produced by transfection of packing plasmids and a transfer plasmid with the gene of interest in adherent or suspension HEK293 cells. Following virus production, mammalian cells and debris are removed from the cell culture fluid (CCF) by centrifugation and/or microfiltration. After clarification, different purification processes are employed, including tangential flow filtration (TFF), size exclusion chromatography (SEC), ultracentrifugation, PEG precipitation, and anion exchange chromatography (AEX) on membranes, nanofibers, monoliths, or resins. These unit operations, however, suffer from long process time and low scalability as well as limited recovery of LVV particles and host cell proteins (HCPs) removal.

The recent consolidation of an industrial layout for manufacturing of adeno-associated viruses (AAV), inspired by the downstream process platform established for monoclonal antibodies, highlights the role of affinity chromatography as the keystone in the purification of viral vectors for cell and gene therapies. For AAVs, a number of affinity adsorbents are available including pan-selective resins as well as serotype-specific resins. These resins offer excellent product yield (50-60%) and purity, but require harsh elution conditions (pH˜2-2.5) that can cause product degradation and aggregation, resulting in a loss of transduction activity. Conversely, the commercial processes for LVV purification are less developed. Heparin has been applied as a pseudo-affinity ligand with reported values of recovery ˜20-50% and 95% removal of HCPs. However, heparin is extracted from animal tissues, which poses concerns of contamination, thus discouraging its use in Good Manufacturing Processes (GMP) processes; moreover, heparin-based adsorbents are not compatible with typical cleaning in place (CIP) procedures used in biomanufacturing, as they rapidly lose selectivity and over 50% of their binding capacity after the first caustic cleaning. Other affinity strategies described in the literature rely on the expression of affinity tags on the LVV envelope. Histidine-tagged LVV particles enable purification via immobilized metal affinity chromatography: monoliths functionalized with sodium iminodiacetate and nickel showed 69% elution efficiency, but modest binding capacity (6.7×10⁸ cell-transducing LVV units per mL of adsorbent, TU/mL). However, the use of imidazole in the elution buffer causes virus inactivation. In another approach, streptavidin-functionalized magnetics beads have been used for purification of LVV particles displaying a cTag8, obtaining yields >60%, and 3-log and 2-log reduction of host cell DNA and HCPs, respectively; however, yield reduced to 20% when the ligands were transferred onto monoliths. More recently, a library of single-domain camelid antibodies (V_{H}H) was used to identify ligands targeting the Vesicular Stomatitis Virus G (VSV-G) protein. The ligands, now commercialized in a chromatographic resin format (CaptureSelect™ Lenti VSVG Affinity Matrix), provide a binding capacity of ˜10¹¹ viral particles per mL (vp/mL) of resin and afford good product purity; however, they require elution conditions (0.8 M arginine) that potentially denature the product and cannot withstand limited caustic (10-25 mM aqueous NaOH) CIP processes.

### SUMMARY

Embodiments of the present disclosure include peptides for purifying a lentivirus from a sample. In some embodiments, the peptides comprise at least one cationic amino acid and/or at least one anionic amino acid; at least one aromatic amino acid; and one or more aliphatic amino acids.

In some embodiments, the peptide is 5 to 20 amino acids in length. In some embodiments the peptide is at least 8 amino acids in length. In some embodiments, the peptide is 8 amino acids in length. In some embodiments, the peptide is 12-20 amino acids in length.

In some embodiments, the peptide comprises at least one cationic amino acid and at least one anionic amino acid. In some embodiments, a cationic amino acid is adjacent to an anionic amino acid.

In some embodiments, each of the at least one cationic amino acid is individually selected from histidine, lysine, and arginine. In some embodiments, the at least one cationic amino acid is lysine.

In some embodiments, each of the at least one anionic amino acid is individually selected from aspartic acid and glutamic acid. In some embodiments, the at least one anionic amino acid is glutamic acid.

In some embodiments, each of the at least one aromatic amino acid is individually selected from histidine, phenylalanine, tyrosine, and tryptophan. In some embodiments, the at least one aromatic amino acid is phenylalanine. In some embodiments, the at least one aromatic amino acid is histidine.

In some embodiments, the one or more aliphatic amino acids are selected from alanine, glycine, isoleucine, leucine, proline, and valine.

In some embodiments, the peptide does not comprise asparagine, glutamine, and/or tryptophan.

In some embodiments, the peptide is a cyclic peptide. In some embodiments, the cyclic peptide is cyclized via a disulfide bond between two cysteine residues.

In some embodiments, the peptide comprises an amino acid sequence having at least 80% sequence identity with one of SEQ ID NOs: 1-86. In some embodiments, the peptide comprises an amino acid sequence having 1, 2, 3, 4, or 5 substitutions as compared with one of SEQ ID NOs: 1-86. In some embodiments, the peptide comprises an amino acid sequence of any of SEQ ID NOs: 1-86.

In some embodiments, the peptide further comprises a linker. In some embodiments, the linker is bound to the C-terminus of the peptide, and wherein the linker is a glycine-rich linker.

In some embodiments, the lentivirus is a Vesicular Stomatitis Virus glycoprotein (VSV-G)-pseudo-typed lentivirus.

In some embodiments, the peptide binds the VSV-G envelope protein. In some embodiments, the peptide targets a binding site of the low-density lipoprotein receptor LDL-R on VSV-G.

Embodiments of the present disclosure also include compositions for purifying a lentivirus from a sample comprising at least one peptide as disclosed herein.

In some embodiments, the at least one peptide is bound to a solid support. In some embodiments, the solid support comprises a non-porous or porous particle, a membrane, a plastic surface, a fiber or a woven or non-woven fibermat, a hydrogel, a microplate, a monolith, and/or a microfluidic device. In some embodiments, the solid support comprises polymethacrylate, polyolefin, polyester, polystyrene, polysaccharide, polyvinyl ether, iron oxide, silica, titania, and/or zirconia.

Further embodiments of the present disclosure provide adsorbents comprising at least one peptide or a composition as disclosed herein. In some embodiments, the adsorbent has a binding capacity of at least 10⁸ cell-transducing LVV units per mL of adsorbent (TU/mL). In some embodiments, the adsorbent has a binding capacity of at least 10⁹ cell-transducing LVV units per mL of adsorbent (TU/mL).

In some embodiments, the adsorbent affords a productivity of at least 10⁸ cell-transducing LVV units per mL of adsorbent per minute of purification time (TU/mL·min). In some embodiments, the adsorbent affords a productivity of at least 10⁹ cell-transducing LVV units per mL of adsorbent per minute of purification time (TU/mL·min).

Additional embodiments of the present disclosure include methods for purifying a lentivirus from a sample. In some embodiments, the methods comprise contacting at least one peptide, a composition, or an adsorbent as disclosed herein, with a sample comprising the lentivirus, wherein the at least one peptide binds the lentivirus; and eluting the lentivirus from the at least one peptide.

In some embodiments, the sample is a biological fluid. In some embodiments, the biological fluid is a cell culture fluid. In some embodiments, the biological fluid comprises a supernatant and/or a cellular lysate. In some embodiments, the biological fluid is derived from a virus production cell line.

In some embodiments, the lentivirus is a Vesicular Stomatitis Virus glycoprotein (VSV-G)-pseudo-typed lentivirus.

In some embodiments, the elution is performed at a pH from about 6.0 to about 8.0. In some embodiments, the method further comprises a washing step before eluting the lentivirus.

In some embodiments, the method produces at least a 100-fold reduction in host cell proteins.

In some embodiments, the method results in at least a 35% yield for cell-transducing LVV units.

In some embodiments, the method results in a productivity of at least 10⁸ cell-transducing LVV units per each milliliter of adsorbent used and per minute of purification time.

Embodiments of the present disclosure also include a lentivirus purified using any of the methods described herein. In some embodiments, the lentivirus exhibits at least 35% transduction activity.

### BRIEF DESCRIPTION OF THE DRAWINGS

FIGS. 1A-1K: Identification of candidate LV-binding peptide sequences. (A) A screening mix was initially formulated containing red-fluorescently labeled HEK293 HCPs at the titer of 0.3 mg/mL and either green-fluorescently labeled LVVs at the titer of ˜10⁹ TU/mL or green-fluorescently labeled VSV-G proteins (^{E}VSV-G and ^{FL}VSV-G) at the titer of 0.2 mg/mL; (B) aliquots of 100 μL of 8-mer peptide-ChemMatrix library beads equilibrated in 20 mM PBS with 75 mM NaCl at pH 6.5 were incubated with 250 μL of screening mix for 30 minutes at room temperature; (C) the beads were washed and individually fed to a microfluid bead sorting device connected to a fluorescent microscope, wherein (D) each bead that displays high green fluorescence emission and green-to-red signal ratio was retained, whole all other beads were discarded; (E) every retained bead was washed with 20 mM citrate buffer with 0.5 M MgCl₂ pH 6.0 for 5 minutes and imaged again; (F) beads that did not show loss of green fluorescence emission were discarded, whereas (G) beads that lost fluorescence signal were collected in 96-well plates; (H) after washing with 0.1 M glycine pH 2.5, water and 30% acetonitrile in water (v/v), the collected beads were individually analyzed by Edman degradation on PPSQ-33A protein sequencer (Shimadzu, Kyoto, Japan) to identify the peptide sequences carried thereupon; finally, (I) the sequences identified by screening the library against full LVV particles, ^{E}VSV-G ectodomain, and ^{FL}VSV-G FL were grouped in homology plots via Weblogo.

FIG. 2: Values of LVV recovery measured via p24 ELISA (total particles), qPCR (total encapsidated transgenes), and transduction assay in HT1080 cells (cell-transducing LVV units), and clearance of HEK293 HCPs (orange histogram) obtained by purifying LVVs from a HEK293 CCF (LVV titer˜10¹⁰ vp/mL, corresponding to ˜10⁸ TU/mL; HCP titer˜0.3 mg/mL) using GKEAAFAA-Poros (SEQ ID NO: 3) resins; the loading step was operated at the RT of 1 minute. The values of yield were measured via p24 ELISA (green histogram), RT-qPCR (blue histogram), and transduction activity on HT1080 cells (teal histogram).

FIGS. 3A-3H: Complexes formed by VSV-G with peptides (A)C-cyclo[GSRQFVADSDRD]C-GSG (red; SEQ ID NO: 87), (B)C-cyclo[GSRSFVGDSDRD]C-GSG (magenta; SEQ ID NO: 88), (C) FEKISAAE-GSG (green; SEQ ID NO: 89), (D) FEKISNAE-GSG (blue; SEQ ID NO: 90), (E) GKEAAFAA-GSG (teal; SEQ ID NO: 91), (F) SRQFVCDSDRD-GSG (yellow; SEQ ID NO: 93), (G) SRTFVCDSDRD-GSG (orange; SEQ ID NO: 95), and (H) SVFRIGLSD-GSG (mustard; SEQ ID NO: 97). The VSV-G and LDL-R are presented as grey and wheat cartoons, respectively; the interacting amino acids on VSV-G and LDL-R are in dark blue and red, respectively. The putative binding sites identified via druggability study of the solvent-accessible surface of VSV-G are presented as wheat, light blue, light green, and light brown surfaces.

FIGS. 4A-4D: Breakthrough curves obtained by loading a HEK293 CCCF (LVV titer˜10¹⁰ vp/mL, corresponding to ˜10⁸ TU/mL; HCP titer˜0.3 mg/mL) on (A) FEKISNAE- (SEQ ID NO: 1), (B) GKEAAFAA- (SEQ ID NO: 3), (C) SRAFVGDADRD- (SEQ ID NO: 4), (D) SFVRIGLSD-Poros (SEQ ID NO: 5) resins at the residence time of either 1 or 2 min. The LVV titer in the effluent was measured via real time qPCR (vg/mL) and transduction assay (TU/mL).

FIGS. 5A-5D: Caustic stability study of (A) GKEAAFAA- (SEQ ID NO: 3), (B) FEKISAAE- (SEQ ID NO: 2), (C) SRAFVGDADRD- (SEQ ID NO: 4), (D) SFVRIGLSD-Poros (SEQ ID NO: 5) resins conducted as consecutive cycles of LVV purification from the HEK293 CCF with intermediate CIP with 0.5 M NaOH (15 CVs at the RT of 1 min followed by 30 minutes of static contact time).

FIG. 6: Relative infectivity of LVV samples in chromatographic buffers. LVV particles were incubated at the initial titer of 5.0 10⁷ TU/mL in citrate-, phosphate-, and histidine-based buffers with different added salts (NaCl or MgCl₂ at different concentrations) and pH for 30 minutes at 25° C. The relative infectious titers are calculated as the ratio of the transduction activity in HT1080 cells of the LVV samples relative to that of LVV particles in DMEM medium.

FIG. 7: Calibration curve for the determination of the surface density of primary amine groups on modified Poros resin. The data points were obtained by analyzing aqueous solutions of ethanolamine at different concentrations via Kaiser (ninhydrin) test.

FIGS. 8A-8E: LVV purification from a clarified HEK293 cell culture harvests. Chromatograms obtained by purifying LVV particles in bind-and-elute mode from a HEK293 CCCF (LVV titer˜10¹⁰ vp/mL, corresponding to ˜10⁸ TU/mL; HCP titer˜0.3 mg/mL) using peptide-based adsorbents (A) GKEAAFAA- (SEQ ID NO: 3), (B) EHFEHWSE- (SEQ ID NO: 12), (C) EWKAAFIW- (SEQ ID NO: 8), (D) FEKISNAE- (SEQ ID NO: 1), and (E) SKSAAEHE-Poros (SEQ ID NO: 6) resins. The equilibration and washing steps were conducted using 50 mM PIPES buffer with 100 mM NaCl at pH 7.4 (RT: 1 min); elution was conducted using a 50 mM PIPES buffer with 0.65 M NaCl at pH 7.4 (RT: 1 min).

FIGS. 9A-9B: Complexes formed by VSV-G with the (A) CR2 and (B) CR3 domains of LDL-R (PDB IDs: 5OYL and 5OY9, respectively). The VSV-G and LDL-R are presented as light blue and wheat cartoons, respectively; the interacting amino acids on VSV-G and LDL-R are in dark blue and red, respectively.

FIG. 10: AKTA chromatograms obtained by loading clarified HEK293 cell culture fluids containing Cas9/GFP-LVV (LVV titer: 2.02·10⁶ TU/mL; HCP titer: 0.05 mg/mL) on GKEAAFAA-Poros™ (SEQ ID NO: 3) resin. The resins were packed in 1 mL column, equilibrated with 100 mM NaCl in 50 mM PIPES buffer at pH 7.4, and loaded with 150 mL of fluid at the RT of 1 min; following washing, LVV elution was conducted using 0.650 M NaCl in 50 mM PIPES at pH 7.4 at RT of 1 min. The UV absorbance of the effluent was monitored at 280 nm.

FIGS. 11A-11B: Caustic stability study of GKEAAFAA-Poros™ (SEQ ID NO: 3) resins conducted as consecutive cycles of LVV purification from the HEK293 CCF with intermediate CIP with 0.5 M NaOH (15 CVs at the RT of 1 min followed by 15 min of static contact time). (A) amount of virus bound to resin and HCP removal; (B) process yield determined by transduction assay with HT1080 cells and RT-qPCR.

FIGS. 12A-12B: Titer of cell-transducing LVV units (TU/mL) measured by transduction assay (histograms) and HCP titer (red dots) measured via ELISA obtained via transient transfection of HEK293 cells cultured in three cell culture media—namely Peak Expression, BalanCD, and LV-Max—using GOI plasmids (A) pALD-LentiEGFP-K and (B) dCAS9-VP64-GFP.

FIG. 13: Comparison of three transfection reagents—namely, TransIT, PEIpro, and LV-Max transfection—for producing LVVs via transient transfection of plasmids pALD-LentiEGFP-K, pALD-Rev-K, pALD-VSV-G-K, and pALD-GagPol-K in HEK293F cells cultured in BalanCD media. Cells were transfected with TransIT and LV-Max upon reaching the density of 4·10⁶ cells/mL (viability >98%), whereas cells were transfected with PEIpro upon reaching the density of 2.5·10⁶ cells/mL (viability >98%). The plasmid:transfection reagent ratio (m/v) and total plasmid concentration per mL of cell culture were (A) 1:3 and 1 μg/mL; (B) 1:1.85 and 1.6 μg/mL; (C) 1:3 and 3 μg/mL; (D) 1:3 and 1.6 μg/mL; (E) 1:3 and 2.5 μg/mL; (F) 1:1 and 2.5 μg/mL; and (G) 1:2.5 and 2.5 μg/mL.

FIG. 14: Confocal images of GKEAAFAA-Poros™ (SEQ ID NO: 3) beads extracted from the front end of a column loaded with pure LVV particles labeled with Syto 13 green, fluorescent dye.

### DETAILED DESCRIPTION

Lentiviral vectors are rapidly becoming an essential tool for producing lifesaving cell therapies. Their manufacturing technology, however, is in its infancy and can afford limited product volumes, thus limiting the application of these therapies to a small group of patients living in advanced economies. While access to healthcare relies on many factors, introducing biomanufacturing technologies that are productive and robust as well as affordable and scalable is critical towards bringing advanced therapies to fruition to a broader patient population worldwide. Disclosed herein is an ensemble of peptide ligands for the purification of LVVs via affinity chromatography. By integrating criteria of affinity, selectivity, and stability of the peptide sequences under different user-defined conditions, ligands with a unique combination of high binding capacity, clearance of impurities, yield of cell-transducing LVV units, and lifetime were elucidated.

As described further herein, these criteria were applied towards experimental identification or suitable peptides as well as the in silico discovery of VSV-G-pseudo typed targeting peptides. Among the sequences identified via library screening, GKEAAFAA (SEQ ID NO: 3) affords a binding capacity of 5·10 TU per mL of resin, a 60-70% yield of cell-transducing LVV units, and a reduction of HCPs above 200-fold, while also demonstrating stability to caustic cleaning. Similarly, among the sequences designed in silico, alkaline-stable SRAFVGDADRD (SEQ ID NO: 4) and SFVRIGLSD (SEQ ID NO: 5) showed a binding capacity of ˜6.24·10⁹ TU/mL, 38-45% yield, and >200-fold HCP clearance. As short peptides, these ligands can be affordably produced at scale: recent studies indicate that, when manufactured at the 10 kg scale or above, the cost of 8-mer peptides can be as little as $60 per gram. Given that ˜25 grams of the proposed peptides are required to functionalize a liter of resin, the cost-of-goods of the peptide-functionalized resins would range between $7.5-9K per liter, thus providing a competitive alternative to affinity resins that rely on protein ligands.

Additionally, experiments were conducted to optimize the parameters governing the purification performance of the peptide ligands, including the material composition and morphology of the chromatographic matrix, and the ligand density and display. Accordingly, the design of affinity adsorbents constructed were optimized using the lead peptides GKEAAFAA (SEQ ID NO: 3), FEKISNAE (SEQ ID NO: 1), and SRAFVGDADRD (SEQ ID NO: 4) in combination with an ensemble of matrices. Specifically, resins of different materials (i.e., polystyrene divinylbenzene, polymethyl methacrylate, polyvinyl ether, and agarose), particle diameters (45-90 m) pore sizes (40-1000 nm), and functional density (0.02-0.1 mmol of peptide per mL of resin) were sourced. Additionally, membranes of different material (cellulose grafted with functionalized with polyethylenimine (PEI) and Natrix®, an inert polymer web impregnated with a porous polyacrylamide hydrogel), pore diameters (˜0.3 m), and functional density (˜0.1 mmol of peptide per mL of membrane) were also tested. The resultant adsorbents varied widely in terms of binding capacity, and LVV yield and purity. A well-performing peptide-functionalized resin and membrane featured a high capacity (respectively, 5·10⁹ and 7·10⁸ transducing LVV units per mL of adsorbent, TU/mL) and productivity (respectively, 2.9·10⁹ and 4.5·10⁸ TU/mL-min), and afforded a substantial enrichment of cell-transducing LVV units and a reduction of contaminants (110-to-170-fold) in the eluates. As described further herein, GKEAAFAA- Poros™ (SEQ ID NO: 3) resin was integrated in an LVV purification process in 4 steps: (i) clarification and nuclease treatment, (ii) affinity-based capture in bind-and-elute mode, (iii) polishing in flow-through mode, and (iv) formulation via ultra/dia-filtration and sterile filtration. The processes afforded a yield of 33%, residual HCP level of 200 ng per mL, and productivity of 1.25·10¹⁴ active LVV particles per hr and liter of resin. The results provided herein demonstrate the use of this technology for large-scale LVV manufacturing.

### 1. Definitions

Unless otherwise defined, all technical and scientific terms used herein have the same meaning as commonly understood by one of ordinary skill in the art. In case of conflict, the present document, including definitions, will be controlled. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate (Asp, D), glutamate (Glu, E)); basic (e.g., lysine (Lys, K), arginine (Arg, R), histidine (His, H)); non-polar (e.g., alanine (Ala, A), valine (Val, V), leucine (Leu, L), isoleucine (Ile, I), proline (Pro, P), phenylalanine (Phe, F), methionine (Met, M), tryptophan (Trp, W)); uncharged polar (e.g., glycine (Gly, G), asparagine (Asn, N), glutamine (Gln, Q), cysteine (Cys, C), serine (Ser, S), threonine (Thr, T), tyrosine (Tyr, Y)); aliphatic (e.g., alanine (Ala, A), valine (Val, V), leucine (Leu, L), isoleucine (Ile, I), glycine (Gly, G), and in some cases, methionine (Met, M)); and aromatic (histidine (H or His), phenylalanine (F or Phe), tyrosine (Y or Tyr), and tryptophan (W or Trp)).

As used herein, the term “purified” or “to purify” refers to the removal of components (e.g., contaminants) from a sample. For example, viruses are purified by removal of contaminating process-related impurities and product-related impurities. Process-related impurities comprise proteins and nucleic acids produced by the host that expresses the virus; they also comprise viruses other than the target virus. Product-related impurities comprise fragments of the target virus; they also comprise transgenes that are not encapsidated or are incorrectly encapsidated; they also comprise aggregates of the target virus and other proteins and nucleic acids. The removal of these contaminants results in an increase in the percentage of active viruses in the sample.

As used herein, the term “sample” refers to any composition or mixture that contains a target biologic. Samples may be derived from biological or other sources. Biological sources include eukaryotic and prokaryotic sources, such as bacterial, fungal, plant and animal cells, tissues, and organs. The sample may also include diluents, buffers, detergents, and contaminating species, debris and the like that are found mixed with the target biologic. The sample may be “partially purified” (e.g., having been subjected to one or more purification steps, such as filtration steps) or may be obtained directly from a host cell or organism producing the target molecule (e.g., the sample may comprise harvested cell culture fluid).

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related impurities. In some embodiments, the target or target biologic is a viral vector (e.g., lentivirus, adeno-associated virus) or a virus-like particle.

As used herein, the term “host cell” refers to any cell line utilized to produce LVVs.

As used herein, the term “encapsidated transgene” refers to any RNA sequence that encodes for one or multiple products (e.g., proteins).

As used herein, the term “cell-transducing LVV units” refers to any number of active lentiviral vectors in a sample that are necessary to transduce one cell, namely, to cause said cell to express one or multiple proteins encoded by the transgene encapsidated in the lentivirus.

As used herein, the term “host cell protein” or “HCP” refers to any protein produced or encoded by the host cell and unrelated to the intended product. HCPs are generally undesirable in the final drug substance.

As used herein, the term “host cell DNA” refers to any nucleic acid produced by the host cell and unrelated to the intended product. Host cell DNA is generally undesirable in the final drug substance.

As used herein, the term “plasmid DNA” or “pDNA” refers to any nucleic acid utilized to transfect the host cell for producing LVVs. pDNA is generally undesirable in the final drug substance.

As used herein, a “mixture” comprises a target biologic of interest (for which purification is desired) and one or more contaminant or impurity. In some embodiments, the mixture is produced from a host cell or organism that expresses the target biologic (either naturally or recombinantly). Such mixtures include, for example, cell cultures, cell lysates, and clarified bulk (e.g., clarified cell culture supernatant).

### 2. Peptides and Compositions for Purifying Lentivirus

a. Peptides

Current platforms for lentivirus purification suffer from (i) concerns of contamination and inactivation of the product due to sub-optimal process conditions, (ii) inadequate yield, and (iii) high cost and poor reusability. More recently, affinity ligands that provide high binding capacity and product purity have been developed, but they require conditions that may denature the product and limit reuse. In seeking robust affinity ligands for lentivirus purification, experimental and in silico methods were used to develop a selection of peptides that selectively bind lentiviruses, enable their elution under near-physiological conditions, and can be reused multiple times without losing binding strength and selectivity.

The disclosed peptides may be used for the purification of any lentivirus. Lentiviruses are a subfamily of enveloped retroviruses. This family includes, for example, human immunodeficiency viruses (e.g., HIV-1 and HIV-2), simian immunodeficiency virus (SIV), bovine immunodeficiency virus (BIV), feline immunodeficiency virus (FIV), caprine arthritis encephalitis virus (CAEV), and equine infectious anemia virus (EIAV). Lentiviruses have a diploid RNA genome that integrates into the host chromosome as proviral DNA for genome replication. Lentiviral infections are persistent because of the ability to integrate their genome into the host chromosome and their ability to evade host immune responses. Unlike other retroviruses, lentiviruses have the ability to efficiently infect and transduce non-proliferating cells, including for example, terminally differentiated cells.

The undesirable properties of lentiviruses can be removed or modified recombinantly, thereby harnessing the beneficial characteristics for use as a delivery vehicle for therapeutic purposes. Consequently, engineered lentiviruses can be produced that are safe, replication-defective, and self-inactivating while still maintaining the beneficial ability to transduce non-dividing cells and integrate into the host chromosome for stable expression. In some embodiments, the disclosed peptides may be used to purify an engineered or recombinant lentivirus.

The engineered or recombinant lentivirus may be a pseudo-typed lentivirus. The term pseudo-typed, as used herein in the context of viral vectors, refers to the modulation of the cell type specificity of a viral vector by integration of foreign viral envelope proteins. This approach is well known in the art and has been described for example in Bischof et al. (Methods Mol Biol. 2010; 614:53-68). Using this approach, host tropism can be altered and/or the stability of the virus can be decreased or increased.

In some embodiments, the peptides described herein target a Vesicular Stomatitis Virus glycoprotein (VSV-G)-pseudo-typed lentivirus. Lentivirus vectors are often pseudo-typed with vesicular stomatitis virus envelope glycoprotein (VSV-G). VSV-G pseudo-typed lentivirus vectors possess superior mechanical stability, which allows spinoculation and production of high-titer vector stocks. The vesicular stomatitis virus (abbreviated herein as VSV) is also referred to as vesicular stomatitis Indiana virus (VSIV) and is a virus of the Rhabdoviridae family of group V (e.g., negative sense ssRNA) viruses. The virus, its hosts, and the diseases triggered by infection with said virus are well-known in the art. VSV-G is a transmembrane glycoprotein and is known to associate efficiently with immature, non-infectious, envelope-deficient retrovirus-like particles assembled by packaging cells to produce infectious, pseudo-typed viruses in cell-free conditions in vitro. The use of VSV-G for pseudo-typing a lentivirus has been described, e.g., in Burns et al. (Proc Natl Acad Sci USA. 1993; 90(17): 8033-8037).

The peptides may target any binding site found on the lentivirus, e.g., VSV-G pseudo-typed lentivirus. In some embodiments, the peptide binds the VSV-G envelope protein. In some embodiments, the peptide targets a binding site of the low-density lipoprotein receptor LDL-R on VSV-G.

In accordance with these embodiments, the present disclosure provides peptides comprising: at least one cationic amino acid and/or at least one anionic amino acid; at least one aromatic amino acid; and one or more aliphatic amino acids. In some embodiments, the peptide comprises at least one cationic amino acid and at least one negatively charged amino acid.

The at least one cationic amino acid and at least one anionic amino acid may be arranged in any orientation along the length of the peptide. In some embodiments, a cationic amino acid is adjacent to an anionic amino acid. For example, in some embodiments, a cationic amino acid is immediately C-terminal to an anionic amino acid. Alternatively, or in addition, a cationic amino acid is immediately N-terminal to an anionic amino acid. In some embodiments, a cationic amino acid is separated from an anionic amino acid by one or more amino acids.

The at least one cationic amino acid may be any amino acid displaying a positively charged residue, natural or synthetic. In some embodiments, each of the at least one cationic amino acid is individually selected from histidine, lysine, and arginine. In some embodiments, the at least one cationic amino acid is lysine.

The at least one anionic amino acid may be any amino acid displaying a negatively charged residue, natural or synthetic. In some embodiments, each of the at least one anionic amino acid is individually selected from aspartic acid and glutamic acid. In certain embodiments, the at least one anionic amino acid is glutamic acid.

In some embodiments, the peptide further comprises an aromatic amino acid. In some embodiments, each of the at least one aromatic amino acid is individually selected from histidine, phenylalanine, tyrosine, and tryptophan. In certain embodiments, the at least one aromatic amino acid is phenylalanine. In certain embodiments, the at least one aromatic amino acid is histidine.

The peptide further includes one or more aliphatic amino acids. The one or more aliphatic amino acids may be arranged along the peptide in any orientation. For example, one or more aliphatic amino acids may separate the aromatic amino acid from the cationic or anionic amino acid, or the cationic and anionic amino acids. In some embodiments, the one or more aliphatic amino acids may be adjacent to each other, thus creating a string of two or more aliphatic amino acids along the length of the peptide. In some embodiments, the one or more aliphatic amino acids are selected from alanine, glycine, isoleucine, leucine, proline, and valine.

In some embodiments, the peptide does not include asparagine, glutamine, tryptophan, or any combination thereof. In some embodiments, the peptide does not include asparagine. In some embodiments, the peptide does not include glutamine. In some embodiments, the peptide does not include tryptophan.

In some embodiments, the peptide has an amino acid sequence having at least 80% sequence identity with one of SEQ ID NOs: 1-86. In some embodiments, the peptide has an amino acid sequence having at least 85% sequence identity with one of SEQ ID NOs: 1-86. In some embodiments, the peptide has an amino acid sequence having at least 90% sequence identity with one of SEQ ID NOs: 1-86. In some embodiments, the peptide has an amino acid sequence having at least 95% sequence identity with one of SEQ ID NOs: 1-86. In some embodiments, the peptide has an amino acid sequence having one of SEQ ID NOs: 1-86.

In some embodiments, the peptide has an amino acid sequence having at least 80% sequence identity with one of SEQ ID NOs: 1-12. In some embodiments, the peptide has an amino acid sequence having at least 85% sequence identity with one of SEQ ID NOs: 1-12. In some embodiments, the peptide has an amino acid sequence having at least 90% sequence identity with one of SEQ ID NOs: 1-12. In some embodiments, the peptide has an amino acid sequence having at least 95% sequence identity with one of SEQ ID NOs: 1-12. In some embodiments, the peptide has an amino acid sequence having one of SEQ ID NOs: 1-12.

In some embodiments, the peptide has an amino acid sequence having at least 80% sequence identity with one of SEQ ID NOs: 1-5. In some embodiments, the peptide has an amino acid sequence having at least 85% sequence identity with one of SEQ ID NOs: 1-5. In some embodiments, the peptide has an amino acid sequence having at least 90% sequence identity with one of SEQ ID NOs: 1-5. In some embodiments, the peptide has an amino acid sequence having at least 95% sequence identity with one of SEQ ID NOs: 1-5. In some embodiments, the peptide has an amino acid sequence having one of SEQ ID NOs: 1-5.

In some embodiments, the peptide has an amino acid sequence having at least 80% sequence identity with at least one of SEQ ID NOs: 1, 3, and 4. In some embodiments, the peptide has an amino acid sequence having at least 85% sequence identity with at least one of SEQ ID NOs: 1, 3, and 4. In some embodiments, the peptide has an amino acid sequence having at least 90% sequence identity with at least one of SEQ ID NOs: 1, 3, and 4. In some embodiments, the peptide has an amino acid sequence having at least 95% sequence identity with at least one of SEQ ID NOs: 1, 3, and 4. In some embodiments, the peptide has an amino acid sequence of at least one of SEQ ID NOs: 1, 3, and 4.

In some embodiments, the peptide has an amino acid sequence having 1, 2, 3, 4, or 5 substitutions as compared with at least one of SEQ ID NOs: 1-86. In some embodiments, the peptide has an amino acid sequence having 1, 2, 3, 4, or 5 substitutions as compared with at least one of SEQ ID NOs: 1-12. In some embodiments, the peptide has an amino acid having 1, 2, 3, 4, or 5 substitutions as compared with at least one of SEQ ID NOs: 1-5. In some embodiments, the peptide has an amino acid having 1, 2, 3, 4, or 5 substitutions as compared with at least one of SEQ ID NOs: 1, 3, and 4.

An amino acid “substitution” or “replacement” refers to the replacement of one amino acid at a given position or residue by another amino acid at the same position or residue within a polypeptide sequence. The amino acid replacement or substitution can be conservative, semi-conservative, or non-conservative. The phrase “conservative amino acid substitution” or “conservative mutation” refers to the replacement of one amino acid by another amino acid with a common property (e.g., aromaticity, charge, polarity, etc.). A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids with common properties, for example, lysine for arginine and vice versa such that a positive charge may be maintained, glutamic acid for aspartic acid and vice versa such that a negative charge may be maintained, serine for threonine such that a free—OH can be maintained, and glutamine for asparagine such that a free—NH₂ can be maintained. “Semi-conservative mutations” include amino acid substitutions of amino acids within the same broad group (e.g., aliphatic), but not within the same sub-group (e.g., polar or non-polar). For example, the substitution of aspartic acid for asparagine, or asparagine for lysine. “Non-conservative mutations” involve amino acid substitutions between different groups, for example, lysine for tryptophan, or phenylalanine for serine, etc.

The peptides may be any length that confers specificity for the target lentivirus. In some embodiments, the peptide is 5 to 20 amino acids in length. In some embodiments, the peptide is 5 to 10 amino acids in length. In some embodiments, the peptide is 5 to 15 amino acids in length. In some embodiments, the peptide is 8 to 10 amino acids in length. In some embodiments, the peptide is 8 to 15 amino acids in length. In some embodiments, the peptide is 8 to 20 amino acids in length. In some embodiments, the peptide is 10 to 15 amino acids in length. In some embodiments, the peptide is 10 to 20 amino acids in length. In some embodiments, the peptide is 12 to 20 amino acids in length. The peptide may be at 5, 6, 7, 8, 9, 10, 11, 12, 13, 14, 15, 16, 17, 18, 19 or 20 amino acids in length. In certain embodiments, the peptide is 8 to 14 amino acids in length. In certain embodiments, the peptide is 8 amino acids in length.

In some embodiments, the peptide is a cyclic peptide. The peptides can be cyclized by any method available to one of skill in the art. For example, the N-terminal and C-terminal ends can be condensed to form a peptide bond by known procedures. Functional groups present on the side chains of amino acids in the peptides can also be joined to cyclize the peptides of the invention. For example, functional groups that can form covalent bonds include —COOH and —OH; —COOH and —NH₂; —COOH and —SH; —SH and —SH. Pairs of amino acids that can be used to cyclize a peptide include, Asp and Lys; Glu and Lys; Asp and Ser; Glu and Ser; Asp and Thr; Glu and Thr; Asp and Cys; Glu and Cys; and Cys and Cys. Other examples of amino acid residues that are capable of forming covalent linkages with one another include cysteine-like amino acids such Cys, hCys, β-methyl-Cys and Penicillamine (Pen), a non-proteinogenic an alpha-amino acid having the structure of valine substituted at the beta position with a sulfanyl group, which can form disulfide bridges with one another. Preferred cysteine-like amino acid residues include Cys and Pen. In some embodiments, the peptide is cyclized via disulfide bond between two cysteine residues.

The groups used to cyclize a peptide need not be found in the amino acid of the peptide itself. Examples of functional groups capable of forming a covalent linkage with the amino terminus of a peptide include carboxylic acids and esters. Examples of functional groups capable of forming a covalent linkage with the carboxyl terminus of a peptide include —OH, —SH, —NH₂ and —NHR where R is (C₁-C₆) alkyl, (C₁-C₆) alkenyl and (C₁-C₆) alkynyl.

Preferably, the reaction conditions used to cyclize the peptides are sufficiently mild so as not to degrade or otherwise damage the peptide. Suitable groups for protecting the various functionalities as necessary are well known in the art (see, e.g., Greene & Wuts, 1991, 2nd ed., John Wiley & Sons, NY), as are various reaction schemes for preparing such protected molecules.

Any of the disclosed peptides and related sequences may be included in a cyclized peptide. As such, the disclosed peptide sequences may include additional amino acids or other groups to facilitate cyclization, as described above. In some embodiments, the peptide is cyclized and comprises an amino acid sequence having at least 80% sequence identity with one of SEQ ID NOs: 13-16.

In some embodiments, a peptide from the present disclosure exhibits a disassociation constant (K_{D}) for a lentivirus (e.g., the VSV-G protein) that is less than or equal to about 10⁻³ M. In some embodiments, a peptide from the present disclosure exhibits a disassociation constant (K_{D}) for a lentivirus (e.g., the VSV-G protein) that is less than or equal to about 10⁻⁴ M. In some embodiments, a peptide from the present disclosure exhibits a disassociation constant (K_{D}) for a lentivirus (e.g., the VSV-G protein) that is less than or equal to about 10⁻⁵ M. In some embodiments, a peptide from the present disclosure exhibits a disassociation constant (K_{D}) for a lentivirus (e.g., the VSV-G protein) that is less than or equal to about 10⁻⁶ M.

As would be recognized by one of ordinary skill in the art based on the present disclosure, the peptides provided herein can be conjugated to a linker. In some embodiments, the linker can facilitate display of a peptide onto a solid support, which allows for better capture of a target lentivirus. In other embodiments, the peptides provided herein are not conjugated to a linker, but can still bind to target lentiviruses and be purified from a cell culture fluid through other means. In some embodiments, the one or more peptides comprise a linker on the C-terminus of the peptide. The linking peptides may have virtually any amino acid sequence, bearing in mind that the preferred linkers will have a sequence that results in a generally flexible peptide. Small amino acids, such as glycine, alanine, and serine are generally used in creating a flexible peptide. A variety of different linkers are commercially available and are considered suitable for use, including but not limited to, glycine-serine polymers, glycine-alanine polymers, and alanine-serine polymers. In select embodiments, the linker comprises [Gly-Ser-Gly]n. In certain embodiments, the linker includes GSG and GGG.

b. Compositions and Adsorbents

Also described herein are compositions and adsorbents comprising one or more of the disclosed peptides. In some embodiments, each peptide of the composition or adsorbent is conjugated to a support. Supports may comprise, but are not limited to, particles, beads, plastic surfaces, resins, fibers, and/or membranes. In some embodiments, the solid support comprises a non-porous or porous particle, a membrane, a plastic surface, a fiber or a woven or non-woven fibermat, a hydrogel, a microplate, a monolith, and/or a microfluidic device. In some embodiments, the solid support includes microparticles and/or nanoparticles. In some embodiments, the solid support comprises a hydrogel. In some embodiments, the solid support comprises a membrane.

In some embodiments, the solid support comprises polymethacrylate, polyolefin, polyester, polystyrene, polysaccharide, polyvinyl ether, iron oxide, silica, titania, agarose, and/or zirconia. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polysulfone, polyethersulfone, polyvinyl ether, and polyethyleneglycol. Suitable natural polymers include, but are not limited to, cellulose, agarose, and chitosan. Suitable metal oxides include, but are not limited to, iron oxide, silica, titania, and zirconia.

In certain embodiments, the solid support comprises particles or beads. In some embodiments, the particles or beads have a diameter of about 50 μm, about 60 μm, about 70 μm, about 80 μm, about 90 μm, or about 100 μm. In select embodiments, the solid support comprises porous particles. In some embodiments, the porous particle comprises pores having pore diameters of at least about 50 nm, at least about 60 nm, at least about 70 nm, at least about 80 nm, at least about 90 nm, at least about 100 nm, or more.

The ligand density on the solid support may vary. In some embodiments, the composition or adsorbent has a high ligand density (e.g., greater than 0.05 mmol/mL). In some embodiments, composition or adsorbent has a lower ligand density (e.g., less than 0.05 mmol/mL).

In some embodiments, the composition or adsorbent comprises a single type of support made from a single type of support material, where all of the peptides are conjugated to supports formed of the single type of support material. In these embodiments, the composition or adsorbent may comprise one or more different types of peptides, each conjugated to the single type of support made from the single type of support material. In other embodiments, the composition or adsorbent comprises a plurality of types of support. Each type of support may be made of the same type of support material or different types of support materials. In these embodiments, the composition or adsorbent may comprise one or more different types of peptides, as described further herein, each conjugated to a different type of support. In still other embodiments, the peptides of the composition can be conjugated to a soluble compound, for example stimuli-responsive polymer chains to remove lentiviruses by affinity precipitation.

In some embodiments, an adsorbent from the present disclosure exhibits a binding capacity for a lentivirus that is at least about 10⁷ cell-transducing LVV units per mL of adsorbent (TU/mL). In some embodiments, an adsorbent from the present disclosure exhibits a binding capacity for a lentivirus that is at least about 10⁸ cell-transducing LVV units per mL of adsorbent (TU/mL). In some embodiments, an adsorbent from the present disclosure exhibits a binding capacity for a lentivirus that is at least about 10⁹ cell-transducing LVV units per mL of adsorbent (TU/mL). In some embodiments, an adsorbent from the present disclosure exhibits a binding capacity for a lentivirus that is at least about 10¹⁰ cell-transducing LVV units per mL of adsorbent (TU/mL). In select embodiments, an adsorbent from the present disclosure exhibits a binding capacity for a lentivirus that is about 10⁹ to 10¹⁰ cell-transducing LVV units per mL of adsorbent (TU/mL).

In some embodiments, the adsorbent results in a productivity of at least 10⁷ cell-transducing LVV units per each milliliter of adsorbent used and per minute of purification time. In some embodiments, the adsorbent results in a productivity of at least 10⁸ cell-transducing LVV units per each milliliter of adsorbent used and per minute of purification time. In some embodiments, the adsorbent results in a productivity of at least 10⁹ cell-transducing LVV units per each milliliter of adsorbent used and per minute of purification time.

In some embodiments, the adsorbent results in at least a 100-fold reduction of host cell proteins. In some embodiments, the adsorbent results in at least a 150-fold reduction of host cell proteins. In some embodiments, the adsorbent results in at least a 200-fold reduction of host cell proteins.

### 3. Methods of Use

As described further herein, the present disclosure also provides methods for purifying a lentivirus from a sample. In some embodiments, the sample is a biological fluid. Thus, the present disclosure provides methods from purifying a lentivirus from one or more product- and/or process-related impurities or contaminants.

In some embodiments, the methods include contacting at least one peptide, a composition, or an adsorbent as described herein with a sample comprising the target lentivirus, wherein the at least one peptide ligand binds the target lentivirus. In accordance with these embodiments, the method includes eluting the target lentivirus from the at least one peptide, thereby purifying the target lentivirus. The methods of the present disclosure can further comprise washing the composition or adsorbent to remove one or more product- and/or process-related impurities or contaminants from the target lentiviruses bound to the peptide ligands. In some embodiments, the method can be performed under any binding conditions suitable for use with the peptides, composition or adsorbent, including both static binding conditions and dynamic binding conditions.

As described further herein, the peptides of the present disclosure exhibit advantages over the compositions and methods currently available to purify lentiviruses. For example, currently available ligands that are used to purify lentiviruses use elution conditions that damage the lentivirus products being eluted. In contrast, the peptide ligands of the present disclosure release bound target lentiviruses under gentler conditions, which do not damage the product.

The binding affinity of the peptides, compositions and/or adsorbent for the lentiviruses, as compared to one or more product- and/or process-related impurities or contaminants, can be altered by changes in the following: properties and concentration of the one or more product- and/or process-related impurities or contaminants; the properties and concentration of the host cell proteins; the composition, concentration, and pH of the mixture; and/or the loading conditions and residence time of the contacting and washing steps. Any of these variables can be changed to variables which are suitable according to the methods of the present disclosure and result in increased or decreased binding affinity as required for the present disclosure.

In some embodiments, the contacting step can comprise a low pH buffer of between pH 5-9. In some embodiments, the contacting step can comprise a low pH buffer of between pH 5-8. In some embodiments, the contacting step can comprise a low pH buffer of between pH 5-7. In some embodiments, the contacting step can comprise a low pH buffer of between pH 6-9. In some embodiments, the contacting step can comprise a low pH buffer of between pH 6-8. In some embodiments, the contacting step can comprise a low pH buffer of between pH 6-7. In some embodiments, the contacting step can comprise a low pH buffer of between pH 7-9. In some embodiments, the contacting step can comprise a low pH buffer of between pH 7-8.

In some embodiments, the elution is performed at a pH from about 5.0 to about 8.0. In some embodiments, the elution is performed at a pH from about 5.0 to about 7.5. In some embodiments, the elution is performed at a pH from about 5.0 to about 7.0. In some embodiments, the elution is performed at a pH from about 5.0 to about 6.5. In some embodiments, the elution is performed at a pH from about 5.0 to about 6.0. In some embodiments, the elution is performed at a pH from about 5.0 to about 5.5. In some embodiments, the elution is performed at a pH from about 5.5 to about 8.0. In some embodiments, the elution is performed at a pH from about 6.0 to about 8.0. In some embodiments, the elution is performed at a pH from about 6.5 to about 8.0. In some embodiments, the elution is performed at a pH from about 7.0 to about 8.0. In some embodiments, the elution is performed at a pH from about 7.5 to about 8.0. In some embodiments, the elution is performed at a pH from about 6.0 to about 7.0. In some embodiments, the elution is performed at a pH from about 5.5 to about 7.5.

In some embodiments, the methods of the present disclosure result in at least a 30% yield for the transgene encapsidated in the lentivirus vectors. In some embodiments, the methods of the present disclosure result in at least a 35% yield for the encapsidated transgene. In some embodiments, the methods of the present disclosure result in at least a 40% yield for the encapsidated transgene. In some embodiments, the methods of the present disclosure result in at least a 45% yield for the encapsidated transgene. In some embodiments, the methods of the present disclosure result in at least a 50% yield for the encapsidated transgene. In some embodiments, the methods of the present disclosure result in at least a 55% yield for the encapsidated transgene. In some embodiments, the methods of the present disclosure result in at least a 60% yield for the encapsidated transgene. In some embodiments, the methods of the present disclosure result in at least a 70% yield for the encapsidated transgene. In some embodiments, the methods of the present disclosure result in at least an 80% yield for the encapsidated transgene. In some embodiments, the methods of the present disclosure result in at least a 90% yield for the encapsidated transgene.

In some embodiments, the methods of the present disclosure result in at least a 35% yield for cell-transducing LVV units. In some embodiments, the methods of the present disclosure result in at least a 40% yield for cell-transducing LVV units. In some embodiments, the methods of the present disclosure result in at least a 45% yield for cell-transducing LVV units. In some embodiments, the methods of the present disclosure result in at least a 50% yield for cell-transducing LVV units. In some embodiments, the methods of the present disclosure result in at least a 55% yield for cell-transducing LVV units. In some embodiments, the methods of the present disclosure result in at least a 60% yield for cell-transducing LVV units. In some embodiments, the methods of the present disclosure result in at least a 70% yield for cell-transducing LVV units. In some embodiments, the methods of the present disclosure result in at least an 80% yield for cell-transducing LVV units. In some embodiments, the methods of the present disclosure result in at least a 90% yield for cell-transducing LVV units.

In some embodiments, the methods of the present disclosure result in a productivity of at least 10⁷ cell-transducing LVV units per each milliliter of adsorbent used and per minute of purification time. In some embodiments, the methods of the present disclosure result in a productivity of at least 5×10⁷ cell-transducing LVV units per each milliliter of adsorbent used and per minute of purification time. In some embodiments, the methods of the present disclosure result in a productivity of at least 10⁸ cell-transducing LVV units per each milliliter of adsorbent used and per minute of purification time. In some embodiments, the methods of the present disclosure result in a productivity of at least 5×10⁸ cell-transducing LVV units per each milliliter of adsorbent used and per minute of purification time. In some embodiments, the methods of the present disclosure result in a productivity of at least 10⁹ cell-transducing LVV units per each milliliter of adsorbent used and per minute of purification time. In some embodiments, the methods of the present disclosure result in a productivity of at least 5×10⁹ cell-transducing LVV units per each milliliter of adsorbent used and per minute of purification time.

In some embodiments, the methods of the present disclosure produce at least an 80-fold reduction in host cell proteins when purifying a lentivirus (e.g., as compared to methods in which the peptide ligands of the present disclosure are not used). In some embodiments, the methods of the present disclosure produce at least a 100-fold reduction in host cell proteins. In some embodiments, the methods of the present disclosure produce at least a 150-fold reduction in host cell proteins. In some embodiments, the methods of the present disclosure produce at least a 200-fold reduction in host cell proteins. In some embodiments, the methods of the present disclosure produce at least a 250-fold reduction in host cell proteins.

Embodiments of the present disclosure also include a lentivirus purified using any of the methods described herein. In some embodiments, the lentivirus exhibits at least 35% transduction activity. In some embodiments, the lentivirus exhibits at least 40% transduction activity. In some embodiments, the lentivirus exhibits at least 45% transduction activity. In some embodiments, the lentivirus exhibits at least 50% transduction activity.

In some embodiments, the biological fluid is a cell culture fluid. In some embodiments, the cell culture fluid comprises a supernatant and/or a cellular lysate. In some embodiments, the cell culture fluid is derived from mammalian cell culture. In some embodiments, the cell culture fluid is derived from HEK cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the cell culture fluid is derived from yeast cells. In some embodiments, the cell culture fluid is derived from a virus production cell line. Exemplary virus production cell lines include, but are not limited to, MDCK-S, MDCK-A, Vero cells, LLC-MK2D, PER.C6, EB66, and AGE1.CR cells, HT1080 cells, and HeLa cells, or any derivatives or variants thereof.

### 4. Materials and Methods

Materials. Dimethyl sulfoxide (DMSO), thioanisole, anisole, ethane-1,2-dithiol (EDT), polybrene, citric acid, hydrochloric acid (HCl), magnesium chloride hexahydrate (MgCl₂·6H₂O), phosphate buffer saline at pH 7.4 (PBS), and Kaiser test kit were obtained from MilliporeSigma (St. Louis, MO). N,N′-Dimethylformamide (DMF), dichloromethane (DCM), viral production cells, LV-MAX production medium, LV-MAX transfection kit, LV-MAX Lentiviral Packaging Mix, Opti-MEM Reduced Serum Medium, Vivid Colors™ pLenti6.3/V5-GW/EmGFP Expression Control Vector, Stbl3™ Chemically Competent E. coli, TrypLE™ express enzyme, fetal bovine serum (FBS), 5,5′-Dithio-bis-(2-nitrobenzoic acid), PureLink™ HiPure Plasmid Maxiprep Kit, NHS-AlexaFluor 488 (AF488), NHS-AlexaFluor 594 (NHSAF594), Dulbecco's Phosphate Buffered Saline (DPBS), TaqMan™ Fast Virus 1-Step Multiplex Master Mix, TaqMan™ custom made probe and primers, Purelink Viral RNA/DNA Kit, Turbo DNAse, Proteinase K, CaptureSelect™ Lenti VSVG Affinity Matrix, Poros™ 50 HE Heparin affinity resin, Poros™ 50 OH resin, and high glucose DMEM supplemented with GlutaMAX™ and pyruvate were obtained from ThermoFisher Scientific (Waltham, MA). Fmoc/tBu-protected amino acids, hexafluorophosphate azabenzotriazole tetramethyl uronium (HATU), piperidine, diisopropylethylamine (DIPEA), N-Methyl-2-pyrrolidone (NMP), and trifluoroacetic acid (TFA) were purchased from ChemImpex (Wood Dale, Illinois). T-75 and T-25 cell culture flasks, 96-well culture plates, DNAse/RNAse-free water, and ampicillin were from VWR (Radnor, PA). Shake flasks and Plasmid⁺ media for bacterial growth were from Thomson (Oceanside, CA). Yeast extract, peptone, and granulated agar were purchased from Genesee Scientific (San Diego, CA). The HT1080 cell line was received from American Type Culture Collection (AATC) (Manassas, VA). Tris(hydroxymethyl)aminomethane hydrochloride (Tris-HCl), sodium phosphate monobasic dihydrate, sodium citrate dihydrate, sodium hydroxide (NaOH), and sodium chloride (NaCl) were sourced from Fisher Chemical (Hampton, NH). Aminomethyl ChemMatrix (particle diameter: 75-150 μm, loading: 0.6 mmol per g resin) resin was from PCAS Biomatrix, Inc. (Saint-Jean-sur-Richelieu, Quebec, Canada). The HIV1 p24 ELISA kits was purchased from Abcam (Waltham, MA). The ectodomain and the full-length VSV-G (^{E}VSV-G and ^{FL}VSV-G) were donated by Merck (Darmstadt, Germany).

Plasmids pALD-LentiEGFP-K, pALD-Rev-K, pALD-VSV-G-K, and pALD-GagPol-K were purchased from Aldevron (Fargo, ND); dCAS9-VP64_GFP was a gift from Feng Zhang (Addgene plasmid #61422; http://n2t.net/addgene:61422);²⁰ TransIT-VirusGEN™ Transfection Reagent for LVV production was purchased from Mirus (Madison, WI). Viral production cells derived from HEK 293F, LV-MAX production medium, LV-MAX transfection kit, TrypLE™ express enzyme, fetal bovine serum (FBS), PureLink™ HiPure Plasmid Maxiprep Kit, Syto 13 dye, SulfoLink iodo activated resin, UltraLink iodoacetyl resin, Purelink Viral RNA/DNA Kit, 0.5 M Bond-Breaker TCEP Solution, POROS™ 50 OH Hydroxyl Activated Resin, and high glucose DMEM supplemented with GlutaMAX™ and pyruvate were obtained from ThermoFisher Scientific (Waltham, MA). Trifluoroacetic acid (TFA), N, N′-disuccinimidylcarbonate (DSC), 4-dimethylaminopyridine (DMAP), Fmoc/tBu-protected amino acids, piperidine, diisopropylethylamine (DIPEA), N-Methyl-2-pyrrolidone (NMP), and hexafluorophosphate azabenzotriazole tetramethyl uronium (HATU) were purchased from Chem-Impex (Wood Dale, Illinois). T-75 and T-25 cell culture flasks, 96-well culture plates, DNAse/RNAse free water, 1,4-Piperazinediethanesulfonic acid (PIPES) sesquisodium salt, BalanCD HEK293 medium, isopropanol and ampicillin were sourced from VWR (Radnor, PA). HT1080 cell line was purchased from American Type Culture Collection (AATC) (Manassas, VA). N,N′-Dimethylformamide (DMF), dichloromethane (DCM), sodium hydroxide, sodium chloride, sodium bicarbonate, 0.45 μm polyethersulfone (PES) vacuum filters, iodoacetyl chloride (IAC), acetonitrile, isopropanol, yeast extract, peptone, and granulated agar were obtained from Fisher Chemical (Hampton, NH). HIV1 p24 ELISA Kit was purchased from Abcam (Waltham, MA). HEK293 HCP ELISA kit was acquired from Cygnus (Southpoint, NC). ToyoPearl® amino-750F, ToyoPearl® AF-Amino-650M were obtained from Tosoh bioscience (Tokyo, Japan). Epoxy-activated Eshmuno resins and Natrix® membranes functionalized with GKEAAFAAC, SRAFVGDADRDC and FEKISNAEC were donated by Merck Life Sciences KGaA (Darmstadt, Germany). Prepacked HiTrap Capto Core 700 columns, Peak Expression medium, and regenerated cellulose membranes with nominal pore size of 1 μm, and 50-mm diameter were purchased from Cytiva (Marlborough, MA). Transfection reagent PEIpro was purchased from Polyplus (Illkirch-France). Nalgene 0.2 μm syringe filters made of polyethersulfone (PES), Amiconultra centrifugal filters (100 kDa), ethane-1,2-dithiol, 2-mercaptoethanol, branched polyethylenimine (molecular weight˜25000 g/mol) (PEI), and benzonase were acquired from MilliporeSigma (Burlington, MA). Lyophilized peptides FEKISNAEC and GKEAAFAAC and iodoacetyl-activated agarose resins were obtained from GenScript (Piscataway, NJ).

Production of lentivirus particles (LV). The LVVs were produced using LV-MAX system (ThermoFisher Scientific (Waltham, MA) following the manufacturer's protocol. The plasmid pLenti6.3/V5-GW/EmGFP was transformed in Stbl3 Chemically Competent E. coli cells and selected on LB agar plates supplemented with 100 μg/mL of ampicillin. Selected colonies were grown in Plasmid⁺ media, and the plasmids were extracted and purified using PureLink™ HiPure Plasmid Maxiprep Kit. Suspension HEK293F cells were grown in LV-MAX media and passaged to achieve a final cell density of ˜5.5·10⁶ viable cells/mL. Twelve hours before transfection, the cells were adjusted to a density of 3.5·10⁶ cell/mL, cultured overnight, and diluted to a final concentration of 4.7·10⁶ cells/mL. In a 125 mL flask, 25.5 mL of cell culture suspension were combined with 1.5 mL of LV-MAX supplement. In a 5 mL vial, 1.5 mL of OptiMEM I was mixed with 45 μg of LV-MAX Lentiviral Packaging Mix (a pre-defined mixture of plasmids pLP1, pLP2, and pLP/VSVG) and 30 μg of Vivid Colors™ pLenti6.3/V5-GW/EmGFP Expression Control Vector plasmid. This mixture was slowly added to 1.5 mL of OptiMEM I and 180 μL of transfection reagent, and incubated at room temperature for 10 minutes. This mixture was slowly added to the viral production cells and placed in an incubator at 37° C. and 8% CO₂ under gentle shaking at 125 rpm. After 6 hours, 1.2 mL of LV-MAX enhancer was added to the cell suspension. The LVV particles were harvested after 48 hours by centrifugation at 1300 g for 15 minutes, followed by filtration using 0.45 μm surfactant-free cellulose acetate (SFCA) filters (ThermoFisher Scientific, Waltham, MA). All LVV samples were immediately stored at −80° C. until further use.

Buffer stability studies. The clarified cell culture fluid (CCF) containing LVV particles was buffer exchanged using 7 kDa Zeba micro spin desalting columns using (ThermoFisher Scientific, Waltham, MA) against (i) 20 mM citrate buffer or 20 mM histidine buffer with 75 mM NaCl at the pH of either 6.0, 6.5, or 7.0; (ii) 20 mM PBS with 75 mM NaCl at the pH of either 6.2, 6.5, or 7.0; (iii) 20 mM citrate at pH 6.0 added with either 0.1, 0.25, or 0.5 M MgCl₂; or (iv) DMEM medium. Samples were incubated at room temperature for 30 minutes, followed by serial dilution in DMEM media supplemented with 8 μg/mL of polybrene. The LVV titer in the samples was determined by transduction assay as described below and the infectivity titers were expressed in comparison with LVV samples in DMEM medium.

Fluorescent labeling of LVV particles, ^{E}VSV-G and ^{FL}VSV-G, and HEK293 HCPs. The NHS-ester dyes Alexafluor 594 (NHS-AF594, red) and NHS-Alexafluor 488 (NHS-AF488, green) were initially dissolved in DMSO at the concentration of 10 mg/mL. LVV particles were purified by centrifugation following the procedure described by Jiang et al. (Sci Rep 5, 13875 (2015) and re-suspended in PBS at pH 7.4. The ^{E}VSV-G and ^{FL}VSV-G as well as the HEK293F cell culture fluid were buffer exchanged to PBS at pH 7.4 using Zeba spin desalting columns 7 KDa molecular weight cut-off (MWCO) (ThermoFisher Scientific, Waltham, MA). Aliquots of 100 μL of LVV particles (˜10¹¹ vp/mL; ˜10⁹ TU/mL) or VSV-G protein (0.2 mg/mL) were mixed with 3 μL of dye NHS-AF488, and incubated at room temperature for 1 hour under mild shaking and in the dark. The same procedure was used for labeling HEK293F HCPs (˜0.3 mg/mL) with NHS-AF594. Unreacted dyes were removed by Zeba Dye removal column (ThermoFisher Scientific, Waltham, MA) and the samples were stored at 4° C.

Production and screening of the peptide library. A library of 8-mer linear peptides in the format X₁X₂X₃X₄X₅X₆X₇X₈G was built following the split-couple-and-recombine method on Aminomethyl ChemMatrix resin via Fmoc/tBu chemistry using protected amino acids Fmoc-Ala-OH, Fmoc-Asn(Trt)-OH, Fmoc-Glu-(OtBu)-OH, Fmoc-Gly-OH, Fmoc-His(Trt)-OH, Fmoc-IleOH, Fmoc-Lys(Boc)-OH, Fmoc-Phe-OH, Fmoc-Ser(tBu)-OH, and Fmoc-Trp(Boc)-OH. Library synthesis was automated using a Syro I peptide synthesizer (Biotage, Uppsala, Sweden). Briefly, aliquots of resin in 5 mL reactor vials were combined with 3 equivalents (eq.) of protected amino acid at a concentration of 0.5 M in DMF, 3 eq. of HATU at 0.5 M in DMF, and 6 eq. of DIPEA at 0.5 M in DIPEA. Coupling was performed at 45° C. for 20 min and followed by washing with DMF. After each reaction step, a Kaiser test was performed to verify the completion of amino acid coupling. The Fmoc protection between two successive residues was removed using 20% v/v piperidine in DMF at room temperature. Upon completing chain elongation, the peptide library was deprotected via acidolysis using a cocktail of TFA:thionasole:EDT:anisole (90:5:3:2) for 2 hours at room temperature. The deprotected library was then washed and stored in dry DMF.

Aliquots of 20 μL of peptide library beads were equilibrated with 20 mM phosphate buffer with 75 mM NaCl at pH 6.5 and combined with 200 μL of a screening mix comprising AF594-labeled HEK293T HCPs (˜0.3 mg/mL) and either AF488-labeled LVV (˜10¹¹ vp/mL; ˜10⁹ TU/mL) or AF488-labeled VSV-G protein (^{E}VSV-G or ^{FL}VSV-G, 0.2 mg/mL). After 30 minutes at room temperature, the beads were collected by centrifugation at 5000 g and resuspended in 200 mL of 20 mM phosphate buffer with 75 mM NaCl at pH 6.5. The library beads were screened using a microfluid device developed in prior work and installed on an Olympus IX81 fluorescent microscope (Center Valley, PA) (Chu, W. et al. J Chromatogr A 1635, 461632 (2021), Barozzi, A., et al., Int J Mol Sci. 21, 3769 (2020), Day, K. et al. Bioconjug Chem 30, 3057-3068 (2019), Kilgore, R. et al. J Chromatogr A 1687, 463701 (2023), Prodromou, R. et al. Advanced Functional Materials 31, (2021), Prodromou, R. et al. Advanced Functional Materials 31, (2021), and Chu, W. et al. J Chromatogr A 1679, 463363 (2022). Individual beads were imaged, and the values of green (AF488) and red (AF594) fluorescence emission were recorded; the bead was then washed for 5 minutes with 20 mM citrate buffer with 0.5 M MgCl₂, and imaged to record the new values of green and red fluorescence emission. All values fluorescence emission, emission ratio, and emission reduction were determined in real-time via image analysis using a custom MATLAB code (MathWorks, Natick, MA). The beads that exhibited (i) high green fluorescence emission and red-to-green emission ratio prior to washing and (ii) >75% reduction of green fluorescence emission after washing were isolated, while all other beads were discarded. The selected beads were finally analyzed via Edman degradation using a PPSQ-33A protein sequencer (Shimadzu, Kyoto, Japan) to sequence the candidate peptide ligands.

In silico design VSV-G-binding peptides and evaluation of VSV-G:peptide interactions. The crystal structure of the complex formed by the Vesicular stomatitis virus Glycoprotein G (VSV-G) and the low-density lipoprotein receptor (PDB ID: 5OY9 and 5OYL) was analyzed to identify the paired residues and estimate their contributions to the binding energy. Nine designed sequences—namely, four disulfide-cyclic sequences (C-cyclo[GSRQFVADSDRD]C-GSG (SEQ ID NO: 87), C-cyclo[GSRSFVGDSDRD]C-GSG (SEQ ID NO: 88), C-cyclo[GSRAFVADADRD]C-GSG (SEQ ID NO: 98), C-cyclo[GSRAFVGDAD]C-GSG (SEQ ID NO: 99)) and five linear sequences (SRQFVCGDSDRD-GSG (SEQ ID NO: 100), SRSFVCDSDRD-GSG (SEQ ID NO: 101), SRAFVGDADRD-GSG (SEQ ID NO: 102), AFVGDADRD-GSG (SEQ ID NO: 103), and SFVRIGLSD-GSG (SEQ ID NO: 104))—together with the sequences identified experimentally—namely FEKISNAE-GSG (SEQ ID NO: 90), FEKISAAE-GSG (SEQ ID NO: 89), FEKISTAE-GSG (SEQ ID NO: 105), GKEAAFAA-GSG (SEQ ID NO: 91), and SKSAAEHE-GSG (SEQ ID NO: 106)—were constructed in Avogadro and modeled in GROMACS using the force field GROMOS 54A7: briefly, each peptide sequence was (i) placed in a simulation box with periodic boundary and containing 2,000 TIP3P water molecules; (ii) equilibrated with 10,000 steps of steepest gradient descent; (iii) heated to 300 K in an NVT ensemble for 250 ps using 1 fs time steps; and (iv) equilibrated to 1 atm via a 500-ps NPT simulation with 2 fs time steps. The production runs were then conducted in the NPT ensemble by applying the Nosé-Hoover thermostat (300 K) and the Parrinello-Rahman barostat (1 atm), respectively; the motion equations were integrated using the leap-frog algorithm with steps of 2 fs; the LINCS algorithm was utilized to constrain the covalent bonds; the Lennard-Jones and short-range electrostatic interactions were calculated using cut-off values of 0.8 nm and 1.2 nm; the particle-mesh Ewald method was utilized for the long-range electrostatic interactions; the lists of bonded and non-bonded interactions (cut-off of 1.2 nm) were updated every 2 fs and 6 fs. The structure of VSV-G was prepared using Protein Prep Wizard (PPW, Schrödinger, New York, NY) by adding missing atoms and explicit hydrogens, removing salt ions and small ligands, and optimizing the hydrogen-bonding network. Two ionization states of VSV-G, one at pH 6.0 and one at 7.4—were obtained and subjected to structural minimization using PROPKA. The structures were then analyzed in SiteMap to identify putative peptide binding sites on VSV-G, namely the sites with high S-score (>0.8) and D-score (>0.9). The candidate peptide ligands were docked in silico against VSV-G at pH 6.0 and one at 7.4 using the docking software HADDOCK (High Ambiguity Driven Protein-Protein Docking) v.2.4. The VSV-G residues present within the selected binding sites and the residues X₁X₂[ . . . ]Xₙ on the peptides were denoted as “active”; all surrounding residues were marked as “passive”. Clusters of VSV-G:peptide complexes with Cα RMSD <7.5 Å were ranked using the dMM-PBSA score, and the top complexes were refined via 200-ns MD simulations to estimate the free energy of binding (ΔG_{B}).

Amination and peptide conjugation of Poros™ 50 OH resin. A volume of 10 mL of Poros™ 50 OH resin was initially dried using a stream of nitrogen, washed in DMF, and resuspended in 50 mL of a solution of CDI at 100 mg/mL in DMF. Samples were kept under stirring and at room temperature. After 5 hours, the resin was copiously washed with DMF and dried with a stream of nitrogen. The resin was then mixed with 100 mL of 5% v/v ethylenediamine in DMF, and incubated at 45° C. under shaking at 100 rpm. After 12 hours, the resin was washed with DMF, followed by DCM, dried with nitrogen, and stored at 4° C. The density of primary amine groups on modified Poros 50 resin beads was determined by Kaiser assay: briefly, 10 mg of resin was mixed with 1 mL of DMF, 0.1 mL of KCN in H₂O/pyridine, and 0.1 mL of ninhydrin, placed in boiling water for 5 min, and cooled down to room temperature; the supernatant was diluted 100-fold in DMF and the UV absorbance of the solution measured at 425 nm using a UV-1800 spectrophotometer (Shimadzu, Kyoto, Japan); ethanolamine was used to build a calibration curve. The selected peptide sequences were synthesized on aminated Poros resin following the procedure outlined below using an Alstra automated peptide synthesizer (Biotage, Uppsala, Sweden).

Purification of LV from HEK293 cell culture supernatant using peptide-Poros resins. The peptide-Poros resins prepared as described above and the control CaptureSelect™ Lenti VSVG and Poros™ 50 HE Heparin affinity resins were flow-packed in 1 mL Tricorn 5/50 columns (Cytiva, Marlborough, MA) and installed on an AKTA Avant FPLC system (Cytiva, Marlborough, MA). The resin packing quality was evaluated by measuring the peak symmetry of the conductivity signal generated by a pulse injection of aqueous 1 M NaCl (target value: 1-1.2). The resins were equilibrated with 10 CVs of equilibration buffer (Table 1). The clarified HEK293 CCF (LVV titer˜0.5-2·10⁸ TU/mL; HCP titer˜0.3-0.1 mg/mL; the ranges encompass the variability of LVV activity across different production batches) was loaded on the resins at the residence time (RT) of either 1 or 3.5 minutes. Following load, the resins were washed with 20 CVs of wash buffer, and the bound LVVs were eluted with 9 CVs of elution buffer (Table 1). Following elution, the resins were regenerated using 10 CVs of 0.1 M glycine containing 2 M NaCl at pH 2.0.

TABLE 1
Composition of chromatographic buffers utilized for the purification
of LVVs using peptide-functionalized Poros resins.

| Equilibration buffer | Wash buffer | Elution buffer |
|---|---|---|
| 20 mM phosphate | 20 mM phosphate | 20 mM citrate buffer |
| buffer | 75 mM NaCl at pH 6.5 | 0.5-1.0M MgCl₂ |
| 75 mM NaCl at | with or without | at pH 6.0 |
| pH 6.5 | 50 mM Arginine | |
| 50 mM Tris buffer | 50 mM Tris buffer | 50 mM Tris buffer |
| 130 mM NaCl at | 130 mM NaCl at | 0.65M NaCl at |
| pH 7.4 | pH 7.4 | pH 7.4 |
| 50 mM HEPES buffer | 50 mM HEPES buffer | 50 mM HEPES buffer |
| 100 mM NaCl at | 100 mM NaCl at | 0.65M NaCl at |
| pH 7.4 | pH 7.4 | pH 7.4 |
| 50 mM PIPES buffer | 50 mM PIPES buffer | 50 mM PIPES buffer |
| 100 mM NaCl at | 100 mM NaCl at | 0.65M NaCl at |
| pH 7.4 | pH 7.4 | pH 7.4 |

Measurements of dynamic binding capacity. GKEAAFAA- (SEQ ID NO: 3), FEKISNAE- (SEQ ID NO: 1), FEKISAAE- (SEQ ID NO: 2), and FEKISTAE- (SEQ ID NO: 11), SRAFVGDADRD- (SEQ ID NO: 4), and SFVRIGLSD- (SEQ ID NO: 5) Poros resins prepared as described below and the control CaptureSelect™ Lenti VSVG and Poros™ 50 HE Heparin affinity resins were flow-packed in 1 mL Tricorn 5/50 columns (Cytiva, Marlborough, MA) and installed on an AKTA Avant FPLC system (Cytiva, Marlborough, MA). Following equilibration with 10 CVs of 50 mM PIPES buffer with 100 mM NaCl buffer at pH 7.4, the resins were continuously loaded with clarified HEK293 CCF (LVV titer˜0.5-2·10⁸ TU/mL; HCP titer˜0.3-0.1 mg/mL) at the RT of either 1 or 2 minutes until the LVV titer in the effluent reached 70-80% of the corresponding feedstock titer. The effluent was apportioned in 3-mL fractions, which were analyzed as described below to measure the titer of encapsidated transgenes and cell-transducing LVV units contained therein. The dynamic binding capacity at 10% of breakthrough (DBC_{10%}) was calculated as described in prior work; the void volume of the system was measured via acetone pulse injection and utilized to adjust the value of DBC_{10%}.

Stability of the peptide-Poros resins. GKEAAFAA- (SEQ ID NO: 3), FEKISNAE- (SEQ ID NO: 1), FEKISAAE- (SEQ ID NO: 2), and FEKISTAE- (SEQ ID NO: 11), SRAFVGDADRD- (SEQ ID NO: 4), and SFVRIGLSD- (SEQ ID NO: 5) Poros resins prepared as described below and the control CaptureSelect™ Lenti VSVG and Poros™ 50 HE Heparin affinity resins were flow-packed in 1 mL Tricorn 5/50 columns (Cytiva, Marlborough, MA), and installed on an AKTA Avant FPLC system (Cytiva, Marlborough, MA). Following equilibration with 10 CVs of 50 mM PIPES buffer with 100 mM NaCl buffer at pH 7.4, the resins were loaded with 30 CVs of clarified HEK293 CCF (LVV titer˜0.5-2·10⁸ TU/mL; HCP titer˜0.3 mg/mL) at the RT of 1 min. After washing the resins with 20 CVs of binding buffer, the bound LVVs were eluted with 4 CVs of 50 mM PIPES buffer with 650 mM NaCl buffer at pH 7.4 at the RT of 1 min. Following elution, the resins were regenerated with 10 CVs of 0.1 M glycine containing 2 M NaCl at pH 2.0 and subjected to cleaning-in-place (CIP) with 15 CVs of 0.5 M NaOH at the RT of 1 min followed by a static incubation for 30 minutes. Both regeneration and CIP steps were conducted at the RT of 1 min. An additional cycle of LVV purification from the clarified HEK293 CCF with intermediate CIP was repeated. The chromatographic fractions were analyzed as described below to measure LVV yield and purity.

Analytical characterization of chromatographic fractions. p24 ELISA and HEK293 HCP ELISA. The titer of p24 protein and HEK293 HCPs in the chromatographic samples was measured via ELISA using kits respectively by Abcam (Cambridge, MA) and Cygnus (Southport, NC) following the manufacturer's instructions.

RT-qPCR. The chromatographic samples were initially treated with TurboDNAse followed by RNA isolation using a Purelink Viral RNA/DNA Kit (ThermoFisher Scientific, Waltham, MA). The samples were then combined with TaqMan fast virus, custom TaqMan probe, and the primers listed below, and analyzed using a CFX Duet Real-Time qPCR System (Bio Rad, Hercules, Ca). Plasmid pLenti6.3/V5-GW/EmGFP was used as a standard.

| Primer | DNA sequence |
|---|---|
| Forward | CCCAGTTCCGCCCATTCTC (SEQ ID NO: 113) |
| primer | |
| Reverse | GCCTCGGCCTCTGCATAAATAAA |
| primer | (SEQ ID NO: 114) |
| Probe | ATGGCTGACTAATTTTT (SEQ ID NO: 115) |

DNA quantification. Total amount of double stranded DNA (dsDNA) was measured using Quant-iT™ PicoGreen™ dsDNA Assay Kits (ThermoFisher Scientific, Waltham, MA) following the manufacturer's protocol.

Fluorescence flow cytometry (FFC). HT1080 cells were seeded in a 96-well plate at the density of 7,000 cells/well in high glucose DMEM media supplemented with GlutaMAX™ pyruvate, and 10% v/v FBS. Plates were centrifuged at 900 g for 5 minutes and placed in an incubator at 37° C. and 5% CO₂. The chromatographic fractions containing LVV particles were serially diluted (10×) in DMEM media supplemented with 8 μg/mL of polybrene (without FBS or antibiotics). After 4 hours, the spent cell culture medium in the 96-well plates was replaced with 0.1 mL of diluted samples and incubated for 12 hours. The samples were then replaced with fresh DMEM media supplemented with 10% v/v FBS. After 72 hours, the cells were detached from the plate via incubation with 150 μL of a mixture composed of TrypLE™ Express Enzyme:DPBS (75:25 v:v) for 15 min at 37° C. The fraction of cells expressing GFP (GFP⁺) was quantified using a CytoFlex flow cytometer (Beckman Coulter, Brea, CA) and the number of transduction units per mL (TU/mL) was calculated using Equation 1 (below):

$$Cell - transducing LVV units ( \frac{TU}{mL} ) = \frac{{N}_{H T 1080} \times \% {GFP}^{+}}{V \times D F}$$

Wherein N_{HT1080} is the number of cells incubated with the diluted AAV sample, V is the volume of the diluted AAV sample, and DF is the dilution factor. Each sample was analyzed in triplicate.

Resin functionalization with peptide ligands. The surface functionality of Poros™ and Eshmuno® resins was converted to primary amino groups following the method described in prior work. Sequences GKEAAFAA (SEQ ID NO: 3), GKEAAFAA-G (SEQ ID NO: 107), GKEAAFAA-GSG (SEQ ID NO: 91), GKEAAFAA-GSGSGSG (SEQ ID NO: 108), GKEAAFAA-GSGPGSG (SEQ ID NO: 109), GKEAAFAA-PEG₃ (SEQ ID NO: 3), FEKISNAE (SEQ ID NO: 1) were synthesized on resins using an Initiator⁺ Alstra™ automated peptide synthesizer (Biotage, Uppsala, Sweden). Each amino acid coupling was performed by incubating a solution of 5 equivalents (eq.) of protected amino acid and HATU in dry DMF, and a solution of 6 eq. of DIPEA in NMP, both at the concentrations of 0.5 M, with the chromatographic resin for 20 min at 70° C. (achieved via microwave heating). After each coupling, Fmoc deprotection was achieved using 20% v/v piperidine in DMF for 30 min at room temperature. Final deprotection of the peptide chain was conducted using a cleavage cocktail containing TFA, thioanisole, anisole, and EDT (90/5/3/2) (v/v) for 2 hrs at room temperature. Following deprotection, the resin was washed with DMF, DCM, dried with a stream of N₂, and stored at 4° C. Bromide activated WorkBead resins were initially reacted with 25% (v/v) NH₄OH (1:1 ratio) at room temperature and under gentle mixing.²¹ After 16 hours, aminated resin was washed with 10 CVs of each solvent in the sequence: water, ethanol and acetonitrile before the next step. A mixture composed of 0.717 mL of IAC, 1.112 mL of TEA and 20 mL of ACN was added to 20 mL of aminated resin and reacted at room temperature, gentle mixing and protected from light. After 3 hours, resin was washed with 10 CVs of ACN, acetone and DMF. Unreacted primary amines in the resin were acetylated by reacting 10 mL of resin with 6.6 mL of acetic anhydride and 10.2 mL of DIPEA in 20 mL of NMP for 3 hours at room temperature. Completion of the reaction was converted by negative result from Kaiser assay. The conjugation of cysteine-derivatized peptides on iodoacetyl functionalized resins was conducted as described in prior work.²² Briefly, the resins were rinsed with water and 50 mM Tris, 5 mM EDTA-Na, 25 mM TCEP, pH 8.5 (coupling buffer). Peptides FEKISNAEC (SEQ ID NO: 110) and GKEAAFAAC (SEQ ID NO: 11) were solubilized in coupling buffer at 10 mg/mL and added to the resin at the ratio 2 mL of peptide solution per mL of settled resin. The conjugation reaction was allowed to proceed at room temperature for 2 hrs under end-to-end mixing and was then quenched with 25 mM 2-mercaptoethanol in coupling buffer. The steps of resin activation, peptide conjugation, and quenching were conducted under dark. The resin was thoroughly rinsed with 1 M sodium chloride and water, and finally stored in 20% v/v ethanol at 4° C. The conjugation of GKEAAFAAC (SEQ ID NO: 11) on iodoacetyl-functionalized Poros™ resins was repeated by incubating 21, 52, 104, and 312 mg of peptide per mL of resin. The residual amount of peptide in the coupling solution was measured via quantitative Ellman assay: briefly, 100 μL of coupling solution were combined with 100 μL of a stock solution of 5,5′-Dithio-bis-(2-nitrobenzoic acid) (Ellman's reagent) at 4 mg/mL in 0.1 M sodium phosphate buffer at pH 8.0, containing 1 mM EDTA; the samples were incubated at room temperature for 15 minutes and analyzed via UV spectrophotometry at the wavelength of 412 nm; solutions of GKEAAFAAC (SEQ ID NO: 11) at 0.2, 0.1, 0.05, and 0.025 mg/mL in coupling buffer were used as standards. Finally, the ligan density was determined by mass balance as the difference between the initial and residual amounts of peptide in the coupling solution divided by the volume of iodoacetyl-functionalized Poros™ resins resin.

Amine activation of cellulose membranes. Regenerated cellulose membranes were initially rinsed with DMF and air dried. 100 cm² of total membrane area was immersed in a solution composed of 35 mL of DMF, 1.80 g of DMAP, and 2.24 g of DSC for 3 hours at room temperature and mixing in a rocker. After reaction, membranes were rinsed with DMF, followed by DMSO, and IPA. Membranes were kept in IPA and 4° C. until the next step. Membranes were air dried and aminated by immersion in a solution composed of 10 g of PEI in 90 mL of MilliQ water at room temperature and shaking in a rocker. After 2 hours, membranes were vigorously rinsed with water, rinsed with ACN and functionalized with IAC following the same procedure described herein.

LVV production and harvest. Viral production cells (ThermoFisher Scientific, Waltham, MA) were cultured in BalanCD LV-MAX, or Peak Expression media at 8% CO₂ and 37° C. to reach a density of 3.5-5.5·10⁶ cells/mL for at least 4 passages before transfection. When the PEIpro transfection reagent, the cells were diluted to 1.5·10⁶ cells/mL at 24 hrs before transfection and adjusted to 2.5·10⁶ cells/mL just before transfection. The PEIpro and plasmids were dissolved in DMEM media (10% of total cell culture volume) at a mass ratio of 1:3 (DNA:PEI) and 1 g of total DNA per 1×10⁶ cells, mixed, and incubated for 15 min at room temperature before being added to the cell suspension. When using the LV-Max system, LVVs were produced following the manufacturer's protocol. When using the Mirus transfection reagent, plasmids were initially diluted in a volume of complex forming solution equals to 10% of cell culture volume and plasmid amount of 1.6 μg/mL of cell culture suspension. Following plasmid dilution, transfection reagent at 3 μL per μg of plasmid was added to same vial, mixed by inverting the tubes 5 times and incubated for 15 min at room temperature, before being added to HEK293F cell suspension at 4.0·10⁶ cells/mL. For all three systems, the cells were removed after 48 hrs post transfection via centrifugation at 1300 g for 15 min and the supernatants were treated with 50 U/mL of benzonase and 2 mM MgCl₂ for 30 min at 37° C. Finally, a clarification step was conducted by filtration using 0.45 μm polyethersulfone (PES) vacuum filters. Unless immediately used, all samples were stored at −80° C.

LV purification using peptide-functionalized resins. Resins were flow packed into adjustable Tricorn 5/50 columns to a final volume of 1 mL and equilibrated with 10 column volumes (CVs) of binding buffer (25 mM PIPES, 100 mM NaCl, pH 7.4). A volume of 10-35 mL of clarified feedstock was loaded in down-flow at a linear velocity of 305 cm/hr (corresponding to a residence time, RT, of 1 min). Following resin wash with 20 CVs of binding buffer, LVV elution was conducted in up-flow with 3 CVs of 25 mM PIPES, 650 mM NaCl, pH 7.4 and 3 CVs of 1 M NaCl, pH 7.4. Cleaning-in-Place (CIP) was conducted with 15 CVs of 0.5 M NaOH (aq) followed by static incubation for 15 min. The resin was finally washed with 10 CVs of equilibration buffer to restore neutral pH. All chromatographic steps were conducted at the flow rate of 1 mL/min (RT: 1 min), while continuously monitoring the conductivity, pH, and UV absorbance of the column effluents at 254, 260 and 280 nm. CaptureSelect™ Lenti VSVG affinity resin was operated following manufacturer's instructions.

Confocal Imagining of GKEAAFAA-Poros™ after loading fluorescently-tagged LVV particles. Lentivirus were purified by sucrose gradient ultracentrifugation following method described by Jiang et al. The resulting LVV pellet was suspended to a titer of ˜1·10¹¹ vp/mL in 0.5 mL of 25 mM PIPES, 100 mM NaCl, pH 7.4 overnight at 4° C. Fluorescent LVV labeling was conducted by incubating 2 μL of a solution of 5 mM Syto 13 in DMSO with ˜2·10¹¹ LVV particles for 30 min at room temperature under dark. The excess dye was removed using Pierce Dye Removal columns (ThermoFisher, Ma). Fluorescently-labeled LVVs were loaded onto GKEAAFAA (SEQ ID NO: 3)-Poros™ resin following the procedure described herein. After loading, the beads extracted from the from the front and back ends of the column were imaged using a Leica Stellaris Confocal Microscope (Wetzlar, Germany).

LV purification using peptide-functionalized membranes. Natrix® membranes functionalized with peptides GKEAAFAAC (SEQ ID NO: 111), FEKISNAEC (SEQ ID NO: 112), SRAFVGDADRDC (SEQ ID NO: 112) were punched in disks of 22 mm in diameter and two layers were housed in a 25 mm Whatman filter holder (Cytiva, Marlborough, MA). The membranes were equilibrated with 50 membrane volumes (MVs) of 25 mM PIPES, 100 mM NaCl, pH 7.4 at 10 MV/min, after which 2-5 mL of clarified feedstock was loaded in down-flow at 3 MV/mL. After washing the membranes with 50 MVs of binding buffer, the LVV elution was conducted in up-flow with 50 MVs of 25 mM PIPES, 650 mM NaCl, pH 7.4 at 10 MV/mL. Finally, the membranes were regenerated with 50 MVs of 0.1 M glycine, 2 M NaCl, pH 2.0 and CIP was conducted with 50 MVs of 0.5 M NaOH (aq). Lentivirus purification with MustangQ devices (MV: 0.86 mL) was conducted according to published work: briefly, the membranes were initially equilibrated with 50 MVs of 10 mM histidine, 150 mM NaCl, pH 7.0, loaded with 150 MVs of clarified feedstock, and washed with 60 MVs of binding buffer; LVV elution was performed in three steps, each comprising 20 MVs at 10 MV/min, using 10 mM histidine at pH 7.0 and NaCl concentration of 0.4, 1.0, and 1.5 M. All chromatographic steps were conducted while continuously monitoring the conductivity, pH, and UV absorbance of the column effluents at 254, 260 and 280 nm.

LVV polishing, buffer exchange, and sterile filtration. A 1 mL column packed with CaptoCore700 resin was equilibrated with 10 CVs of 25 mM PIPES, 100 mM NaCl, pH 7.4 at the RT of 1 min and loaded in down-flow with 20 CVs of the elution stream obtained from GKEAAFAA (SEQ ID NO: 3)-POROS™ (LVV titer: 6.2·10⁹ vp/mL; HCP titer: 2.6 μg/mL) at the RT of 2 min. The resin was finally cleaned in up-flow with 15 CVs of 1 M NaOH in 30% (v/v) isopropanol:water followed by 30 min of static contact. All chromatographic steps were conducted while continuously monitoring the conductivity, pH, and UV absorbance of the column effluents at 254, 260 and 280 nm. After polishing, the LVVs were concentrated to a titer of 1.2·10¹⁰ vp/mL by centrifugation in Amicon filters (MWCO: 100 kDa) at 3500 g for 30 min using 10 diavolumes of 25 mM PIPES, 10% sucrose, 20 mM MgCl₂, pH 7.4. Finally, the samples were filtered using Nalgene PES 0.2 μm syringe filters and immediately analyzed or stored at −80° C.

Analysis of chromatographic samples. Transduction assay. HT1080 cells were cultured in DMEM supplemented with 10% FBS at 5% CO₂ and 37° C. until 80-90% confluence was reached. Cells were released from the culture flasks using trypsin, counted using a hemocytometer and trypan blue for cell viability, and plated in a 96-well plate at 7,000 cells/mL. Plates were centrifuged at 900 g for 5 min and kept in an incubator for 4 hrs. At the onset of the transduction assay, the culture media in the plates were replaced with equal volumes of samples prepared via serial dilution (10×) of the fractions containing LVVs in DMEM media supplemented with 8 μg/mL of polybrene. After 12 hrs, the spent medium was replaced with fresh DMEM medium supplemented with 10% v/v FBS, and the cells were incubated for 60 hrs. The fractions of cells expressing GFP were measured using a CytoFLEX flow Cytomer (Beckman, Brea, CA) and the values of transduction units (TU) per mL were calculated using Equation 2 (below). Only dilutions that yielded % GFP⁺ cells between 1% to 25% were considered for LVV transduction concentration.

$$Transduction ( \frac{TU}{mL} ) = \frac{number of cells * ( \% \frac{{GFP}^{+}}{100} )}{volume ( mL ) * dilution factor}$$

where the transductions units (TU) per mL were determined based on the number of cells at the time of transduction, the number of HT1080 cells expressing GFP, the total volume of sample per well, and the dilution factor.

HEK293 HCP ELISA and p24 ELISA. The titer of p24 protein and HEK293 HCPs were respectively measured using HIV ELISA (Abcam, Waltham, MA) and HEK293 HCP ELISA (Cygnus, Southpoint, NC) kits following the manufacturer's instructions. From the values of HCP titer, the reduction values (RVs) and logarithmic reduction values (LRV) of HCPs were derived using the equation below,

$$RV = \frac{{C}_{HCP , E} / {C}_{AAV , E}}{{C}_{HCP , L} / {C}_{AAV , L}}$$

$$LRV = {log}_{10} ( RV )$$

where C_{HCP,E} and C_{HCP,L} are the HCP titers in the elutates and corresponding loads; C_{LVV,E} and C_{LVV,L} are the LVV titers in the elutates and corresponding loads.

Real time quantitative PCR (RT-qPCR). RT-qPCR was conducted as described in prior work. Briefly, DNAse-treated samples were purified using a Purelink Viral RNA/DNA Kit (ThermoFisher Scientific, Waltham, MA) to isolate the encapsidated RNA. The samples were then combined with TaqMan fast virus, custom TaqMan probe, and the primers listed herein, and analyzed using a CFX Duet Real-Time qPCR System (Bio Rad, Hercules, CA). Plasmid pALD-LentiEGFP-K was used as a standard.

### 5. Examples

The accompanying Examples are offered as illustrative as a partial scope and particular embodiments of the disclosure and are not meant to be limiting of the scope of the disclosure.

### Example 1

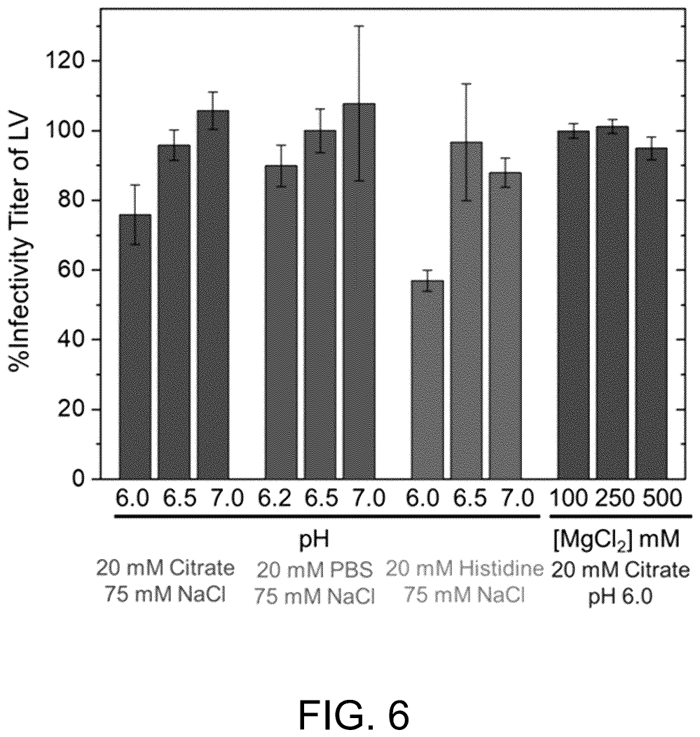

Selection of chromatographic buffers for library screening. Lentivirus particles are highly sensitive to the physicochemical properties of the aqueous environment and their transduction activity can be irreversibly damaged by small variations of pH, salt concentration, temperature, and osmotic pressure. In the context of bioprocessing, this limits the latitude of chromatographic buffers suitable for LVV purification. Particularly stringent is the limitation on elution pH, which, being confined to the range of 6 to 8, cannot be leveraged as in the affinity purification of antibodies and AAVs. Accordingly, different formulations of binding and elution buffers that are compatible with LVV were explored, initially focusing on citrate-, phosphate-, and histidine-based solutions with different ionic strength and pH. Preliminary stability studies conducted by incubating 10⁸ TU/mL LVV particles in the various buffers for 30 minutes (FIG. 6) indicated: (i) no significant loss of infectious titer in citrate and phosphate buffers with 75 mM NaCl at pH 6.0 and 7.0; and (ii) significant reduction in infectivity (>40%) in 20 mM histidine buffer with 75 mM NaCl at pH 6.0. To formulate the elution buffer, the ionic strength was increased using sodium chloride and magnesium chloride, in lieu of decreasing pH. Magnesium chloride in sodium citrate buffer did not affect LVV activity at pH 6.0 at concentrations ranging from 100 to 500 mM (FIG. 6). Based on these results, 20 mM phosphate buffer with 75 mM NaCl at pH 6.5 and 20 mM citrate buffer with 500 mM MgCl₂ at pH 6.0 were chosen as equilibration and elution buffers, respectively, for library screening.

Identification of LV-targeting candidate peptide ligands. The LVV vectors utilized in ex vivo applications, including all FDA-approved therapeutics ABECMA, CARVYKTI, BREYANZI, SKYSONA and ZYNTEGLO, have been designed by pseudotyping. This approach consists in replacing the wildtype envelope glycoprotein gp120, which underlies the HIV virus' tropism of for human CD4⁺ T cells, with heterologous glycoproteins. Most LVV pseudo-typing to date employs the vesicular stomatitis virus G glycoprotein (VSV-G), which endows the vector particles with high stability and the ability to transduce a wide variety of cell types by targeting a ubiquitous cell membrane phospholipid. Other proteins utilized for LVV pseudotyping, namely, the feline endogenous virus (RD114) envelope glycoprotein, the Measles virus hemagglutinin and fusion glycoproteins, the Gibbon ape leukemia virus envelope protein, the Rabies virus glycoprotein, and the Moloney murine leukemia virus 4070A-envelope protein (amphotropic), have not provided comparable efficacy. Accordingly, VSV-G-pseudo-typed LVVs are expected to be utilized in the design of ex vivo cell therapies in the foreseeable future.

Under this premise, two model targets were selected for ligand selection, the single VSV-G protein and mature VSV-G-pseudo-typed LVV particles. The VSV-G protein comprises 3 domains, namely the ectodomain (^{E}VSV-G), which is displayed on the viral surface, the transmembrane domain, which anchors the protein in lipid layer of the viral envelope, and the cytoplastic domain. While in principle only the ^{E}VSV-G can be targeted by surface-immobilized ligands, no information is available on the role of either the transmembrane domain or the intercalation of the full-length VSV-G (^{FL}VSV-G) in the lipid layer on the tertiary structure of the ectodomain. Accordingly, to avoid biasing the ligand selection towards a model target that is not representative of the product, both ^{E}VSV-G and ^{F}VSV-G were adopted for library screening.

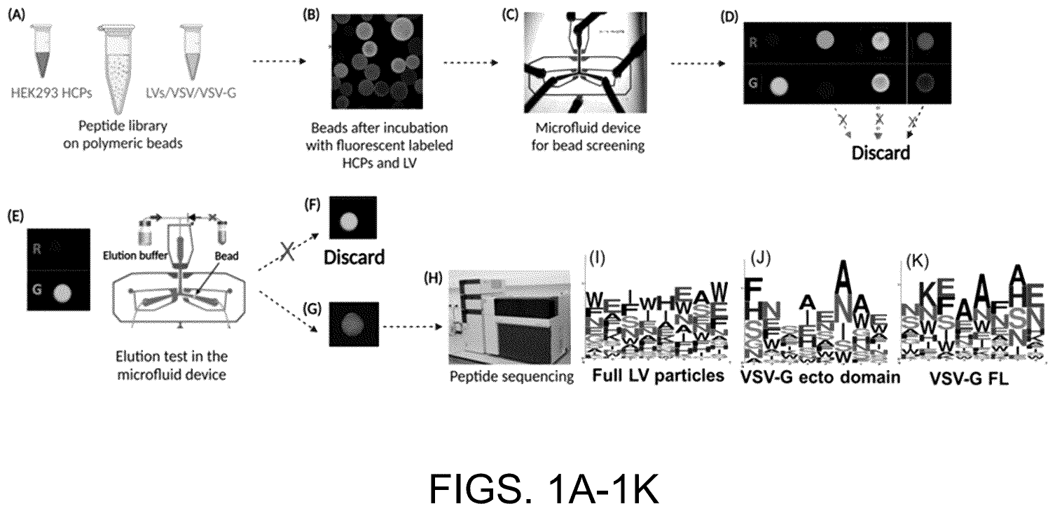

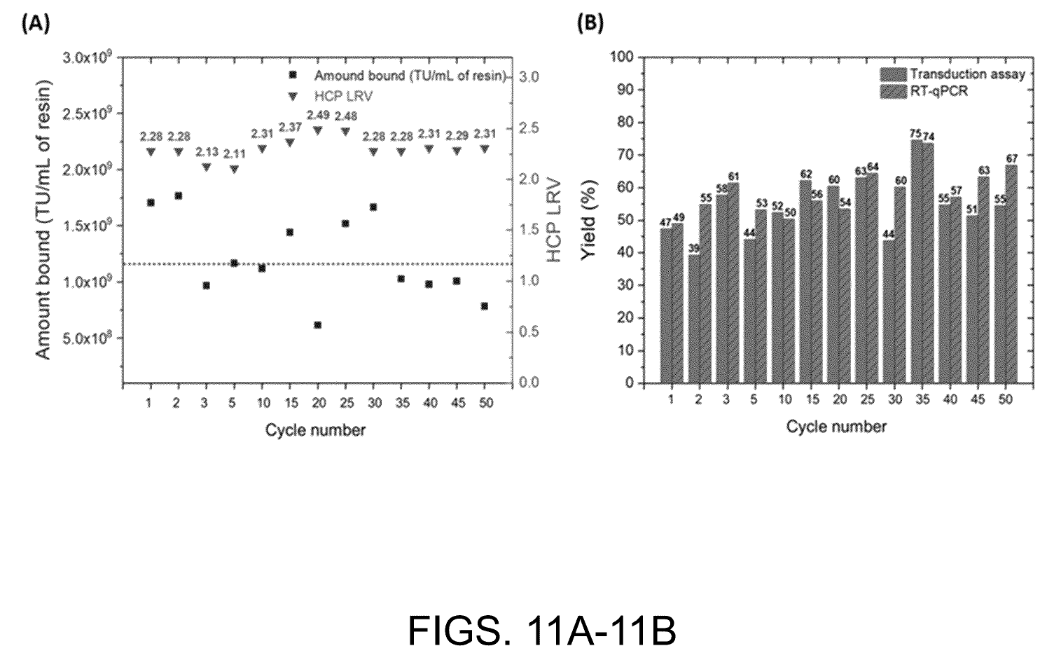

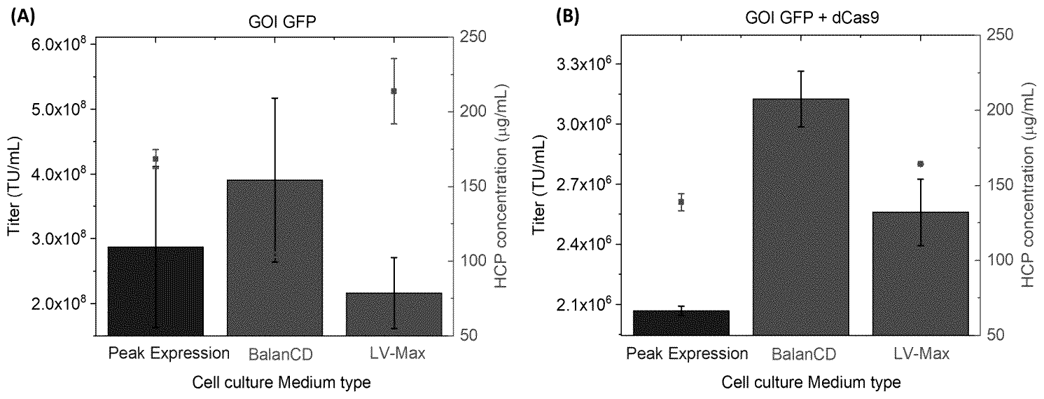

The selection of candidate peptide ligands was initially performed by screening a solid phase peptide library using a device for ligand development. The technology relies on orthogonal fluorescence labeling to ensure the selection of ligands that possess strong and selective binding, but can also release the target when exposed to mild elution conditions. To this end, a microfluidic bead-imaging-and-sorting device was designed and installed in a fluorescence microscope, routinely utilized to implement a protocol for peptide ligand discovery (FIG. 1): (i) the solid-phase peptide library, produced as a one-bead-one peptide library on hydrophilic and translucent porous particles, is incubated with the target labeled with a green fluorescence dye and a multiplicity of impurities, herein, the whole HEK293 cell proteome, collectively labeled with a red fluorescent dye (note: to mimic industrial LVV harvests, the screening mix was formulated with an HCP titer of ˜0.3 mg/mL and either an LVV titer of ˜10⁹ TU/mL or a VSV-G titer (^{E}VSV-G or ^{FL}VSV-G) of 0.2 mg/mL); herein, Alexafluor 488 and Alexafluor 594 were adopted as the green and red dye, respectively, owing to their low propensity to alter the structure and behavior of the labeled protein); (ii) following incubation, the library beads are thoroughly washed and individually fed to the microfluidic device, where they are imaged and the image metrics are analyzed in real time; (iii) a bead that displays high green-only fluorescence, denoting selective and strong target affinity, is withheld in the imaging chamber, while all other beads are ejected; (iv) the bead is then exposed to a flow of selected elution buffer, 20 mM citrate buffer with 0.5 M MgCl₂ pH 6.0, and imaged again; (v) the beads that display a strong loss of green fluorescence, denoting the ability to release the product under elution conditions defined by the operator, are selected, while all other beads are discarded. The steps (i)-(v) are automated by a custom Matlab code, which enables conducting the library screening at a rate of 350 beads per hour. Finally, (vi) the sequences carried by the selected beads are identified via Edman Degradation. The list of the peptides identified against ^{E}VSV-G, ^{FL}VSV-G, and full LVV particles is reported in Table 2, while the sequence homology plots are reported in FIG. 11. The identified sequences are ostensibly amphiphilic, each containing at least one aromatic (Phe or Trp) and multiple aliphatic (Ala, Ile) amino acids. Rather striking in the abundance of Glu, which appears at least once in 72% of the identified sequences and twice in 30% of the sequences. The presence of Glu was unexpected due to the negative charge of the LVV surface, rooted in the phospholipid bilayer forming the envelope, which has made anion exchange chromatography the primary method of separation. At the same time, the hydrophilic and anionic character of the candidate ligands reduces the risk of binding HCPs, which requires peptides rich in cationic and hydrophobic amino acids.

### Example 2

Evaluation of candidate peptide ligands in dynamic conditions. Most of the chromatographic resins on the market feature a pore diameter ranging between 20 and 100 nm. While suitable for the purification of protein-based biologics, these adsorbents are not ideal for large viral vectors like LVVs and baculovirus, whose diameter can reach 100 nm. Accordingly, the bioseparation community envisions that chromatographic substrates with large pore diameters, such as membranes and monoliths, will become mainstream in the downstream processing of viral vectors. In this work the selected peptides were evaluated using established chromatographic adsorbents to avoid uncertainties related to peptide surface density and display. Notably, Poros™ 50 OH resin features pores with diameter of up to 1000 nm and is therefore well suited for the purification of LVV particles.

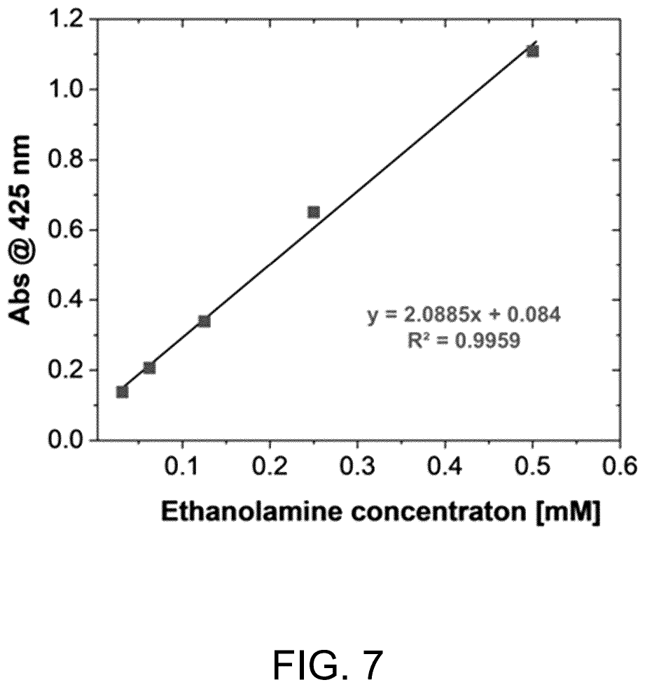

The Poros™ 50 OH beads were modified by converting its hydroxyl groups to primary amino groups, reaching a functional density of 172 μmol per g of resin (FIG. 7), which were utilized for the conjugation of the sequences identified via library screening (Table 2). The resulting peptide-Poros resins were evaluated for LVV binding and elution in dynamic conditions by loading a clarified HEK293 CCF (LVV titer˜10¹⁰ vp/mL, corresponding to ˜10⁸ TU/mL, and HCP titer˜0.3 mg/mL; note: some variability in the titer of total and cell-transducing LVV units was observed across different production batches) at the residence time (RT) of 3.5 min recommended for Poros™ resins and using the selected binding and elution buffers (20 mM phosphate with 75 mM NaCl at pH 6.5 and 20 mM citrate buffer with 0.5 M MgCl₂ at pH 6.0, respectively).

The values of LVV yield and purity listed in Table 3 point at SIEINSSE (SEQ ID NO: 10), GEFENINW (SEQ ID NO: 7), EWKAAFIW (SEQ ID NO: 8), SKSAAEHE (SEQ ID NO: 6), GKEAAFAA (SEQ ID NO: 3), SNEIEIAN (SEQ ID NO: 9), and FEKISNAE (SEQ ID NO: 1) as promising ligand candidates: specifically, these sequences afforded a 10-to-70-fold reduction of HEK293 HCPs and up to 70% reduction of cell DNA; and values of encapsidated transgene yield ranging between 30% and 50%. Given the vulnerability of LVV particles to changes in buffer composition, conductivity, and pH, that often cause a substantial loss of infectivity, the transduction activity of the purified LVVs was evaluated on HT1080 human fibrosarcoma cells as an additional metric to guide the choice of candidate ligands. The values of LVV recovery afforded by the selected sequences, collated in Table 4 along with other purification parameters, demonstrated that FEKISNAE (SEQ ID NO: 1), and GKEAAFAA (SEQ ID NO: 3) perform comparably to control affinity adsorbents Poros™ 50 HE Heparin and CaptureSelect™ Lenti VSVG affinity resins, providing yields of cell-transducing LVV units between 38% and 41%. These values, however, are viewed as rather low when framed in the context of achieving affordable manufacturing of LVVs.

Seeking to improve the values of LVV yield, the effect of residence time of the loading step was first investigated. The amount of clarified HEK293 CCF loaded on the columns, 30 mL calculated based on a reference value of load of ˜3·10¹¹ vp per mL of resin and the LVV titer of ˜10¹⁰ vp/mL in the feedstock, when loaded at the flow rate of ˜0.3 mL/min (RT of 3.5 min), results in a total loading time of 1.75 hrs. Combined with the duration of the harvest and clarification steps, chromatographic washing and elution steps, and incubation of purified LVVs with HT1080 cells, this brings the total process time to about 3 hrs. Comparing this time to the half-life of VSV-G pseudo-typed LVVs at room temperature, estimated at 35-hours, suggested that the values of recovery of cell-transducing LVVs may be negatively impacted by the long processing time. To obviate this inconvenience, additional testing of select peptides was conducted by reducing the residence time of all chromatographic steps from 3.5 to 1 min, which shortened the processing time from 3 hours to about 50 minutes. The sequences GEFENINW (SEQ ID NO: 7) and SNEIEIAN (SEQ ID NO: 9) were selected based on the recovery of LVV particles and transgenes, and FEKISNAE (SEQ ID NO: 1), EWKAAFIW (SEQ ID NO: 8), SKSAAEHE (SEQ ID NO: 6), GKEAAFAA (SEQ ID NO: 3) were selected based on the recovery of cell-transducing LVV units. The results collated in Table 4 show that reducing the load residence time proved beneficial to the performance of all resins: in particular, the yield of FEKISNAL (SEQ ID NO: 1), GEFENJNW (SEQ ID NO: 7), and GKEAAFAA (SEQ ID NO: 3) increased 1.7-to-3-fold, while their HCP clearance grew from a LRV of 1.4-1.6 to 1.8-2.7; notably, the product yield and purity afforded by Poros™ 50 HE Heparin also doubled, indicating that the need of a shorter loading time is not tied to a particular chemical composition of the ligands. It is also worth noticing that the performance of the peptide-based adsorbents was comparable to that of the control affinity resins, in terms of recovered lentiviral particles and transgenes as well as purity. At the same time, with the values of yield well below 50%, further process optimization is necessary to improve the economics of LVV production.

TABLE 2
Sequences and biophysical properties of 8-mer peptides selected via library
screening against mature LVV particles, ^{E}VSV-G, and ^{FL}VSV-G. The sequences were identified
via Edman degradation of the selected library beads. The values of isoelectric point (pI)
and Grand Average Hydropathy index (GRAVY) were calculated based on the amino acid
sequences and assuming an amidated C-terminus to represent the conjugation of the peptide
to the chromatographic resin.

| LVV particles | | | ^{E}VSV-G | | | ^{FL}VSV-G | | |
|---|---|---|---|---|---|---|---|---|
| Sequence | Ip | GRAVY | Sequence | Ip | GRAVY | Sequence | Ip | GRAVY |
| NEAIAWSA | 6.99 | 0.15 | HFGNHAHS | 11.05 | -1.21 | GKEAAFAA | 10.13 | 0.28 |
| SANWAIEW | 6.99 | −0.19 | FEKISNAE | 7.05 | -0.76 | GNSNAAHF | 11.35 | -0.63 |
| FFFWKEWE | 7.05 | −0.54 | SWFHWNGW | 11.15 | -0.98 | FFFNAFAH | 11.30 | 1.01 |
| EKNKEKAN | 10.35 | −2.99 | GWAANWGF | 11.23 | 0.04 | SHIKNSAN | 12.05 | -1.18 |
| WFIIEESG | 4.29 | 0.34 | FGKSAAAA | 12.20 | 0.61 | ANFGAHSK | 12.10 | -0.68 |
| AINNHEWE | 5.31 | 1.48 | SNEIEIAN | 4.29 | -0.50 | KKWAIGSK | 12.70 | 0.94 |
| ENSNHSAW | 7.90 | −1.80 | HENISNSW | 7.90 | -1.46 | EWKAAFIW | 10.13 | 1.21 |
| FEFSEWAW | 4.29 | 0.28 | SIEINSSE | 4.29 | -0.49 | NWEFWSHN | 7.90 | -1.69 |
| NEKWHEAF | 7.88 | −1.74 | NNWHEWHI | 8.00 | -1.78 | NIFHFNSN | 11.25 | -0.55 |
| EHFEHWSE | 5.22 | −1.98 | AFIHEAWS | 7.90 | 0.31 | SKSAAEHE | 7.88 | -1.51 |
| IWEFKNHE | 7.88 | −1.40 | GNSEKAAW | 10.13 | -1.18 | SKSAAEHE | 7.88 | -1.51 |
| WEIAKHSF | 10.13 | −0.40 | HNAWFAAA | 11.40 | 0.30 | NESHINIS | 7.90 | -0.79 |
| LKIWEWEI | 7.05 | 0.01 | FFFAENWE | 4.29 | -0.15 | NWFWSFNE | 6.99 | -0.94 |
| SHFENNIW | 7.90 | −1.01 | SNSEWANI | 6.99 | -0.84 | SKAAAFSH | 12.34 | -0.06 |
| WFWHAAIF | 10.89 | 1.09 | FWSAFINE | 6.99 | 0.40 | WIIAWNHE | 7.90 | -0.15 |
| WLSAAFFH | 10.92 | 1.01 | NEISSSWF | 6.99 | -0.38 | SHFAWASE | 7.90 | -0.35 |
| ESFWFNNE | 4.29 | −1.26 | FSSAAIWN | 11.40 | 0.61 | ASWSENNI | 6.99 | -0.84 |
| WHISHAAN | 11.01 | 0.44 | HAWENNFG | 7.90 | -1.30 | IKEIKENN | 9.86 | 1.60 |
| AFWWGHHF | 10.88 | −0.15 | | | | NNWEAWEN | 4.29 | -2.19 |
| FNNNHEWF | 7.90 | −1.56 | | | | FNEFNKAN | 10.13 | -1.31 |
| WEIWHFEE | 4.59 | −1.03 | | | | WKIEENNE | 4.63 | -2.23 |
| GENNINSN | 6.99 | −1.78 | | | | HWEENWAE | 4.59 | −2.15 |
| FIHHIWFS | 10.91 | 0.81 | | | | | | |
| FWIAIEAI | 6.99 | 1.94 | | | | | | |
| WESIIFAA | 6.99 | 1.28 | | | | | | |
| NKIIWANS | 12.51 | −0.23 | | | | | | |
| NAAFIWNH | 10.89 | −0.03 | | | | | | |
| NKGHSNEE | 7.88 | −2.79 | | | | | | |
| SESIIAWW | 6.99 | 0.49 | | | | | | |
| KAHEHIFW | 10.13 | −0.70 | | | | | | |
| WSSISEGG | 6.99 | −0.39 | | | | | | |
| GHFENINW | 7.90 | −0.96 | | | | | | |
| GASFFISW | 11.45 | 1.13 | | | | | | |
| GSWGGHHW | 11.10 | −1.28 | | | | | | |

TABLE 3
Evaluation of the candidate peptide sequences via LVV purification in
flow. Values of LVV yield (viral particles measured via p24 ELISA and
encapsidated transgenes measured via qPCR), logarithmic removal value
of HEK293 host cell proteins (HCP LRV), and residual double-strand DNA
obtained via chromatographic purification of LVV particles in bind-
and-elute mode from a clarified HEK293 cell culture harvest (LVV
titer: ~10¹⁰ vp/mL, corresponding to ~10⁸ TU/mL; HCP titer: ~0.3 mg/mL)
using the peptides identified by screening the 8-mer peptide-ChemMatrix
library against either (1) mature LVV particles, (2) ^{E}VSV-G, or (3)
^{FL}VSV-G targets, and conjugated on aminated Poros resins; The
equilibration and washing steps were conducted using 20 mM phosphate with
75 mM NaCl at pH 6.5 (RT: 3.5 min); elution was conducted using 20 mM
citrate buffer with 0.5 M MgCl₂ at pH 6.0.

| | | Encapsidated | | |
|---|---|---|---|---|
| | LVV particle | transgene | | |
| | yield | yield | HCP | Residual |
| Sequence | (p24 ELISA) | (qPCR) | LRV | dsDNA |
| GEFENINW¹ | 14% | 51% | 1.62 | 47% |
| EHFEHWSE¹ | 13% | 25% | 1.88 | 29% |
| EWKAAFIW¹ | 12% | 40% | 0.84 | 28% |
| LKIWEWEI¹ | 7% | 26% | 0.58 | 55% |
| IWEFKNHE¹ | 7% | 27% | 1.31 | 21% |
| WFWHAAIF¹ | 3% | 32% | 2.08 | 65% |
| NKGHSHEE¹ | 2% | 25% | 0.79 | 8% |
| SWFHWNGW¹ | 1% | 40% | 0.52 | 52% |
| KAHEHIFW¹ | 1% | 26% | 0.56 | 27% |
| WSSISEGG¹ | 1% | 31% | 2.31 | 74% |
| GSWGGHHW¹ | 1% | 19% | 1.51 | 55% |
| SESIIAWW¹ | 1% | 22% | 1.81 | 42% |
| ESFWFNNE¹ | <1% | 9% | 1.98 | 48% |
| FWIAIEAI¹ | <1% | 10% | 1.81 | 26% |
| GASFFISW¹ | <1% | 2% | 1.59 | 27% |
| NAAFIWNH¹ | <1% | 11% | 2.07 | 9% |
| WFIIEESG¹ | <1% | 11% | 1.83 | 41% |
| WESIIFAA¹ | <1% | 6% | 1.79 | 49% |
| WHISHAAN¹ | <1% | 9% | 1.52 | 54% |
| SNEIEIAN² | 23% | 28% | 1.84 | 40% |
| FEKISNAE² | 16% | 44% | 1.61 | 72% |
| FGKSAAAA² | 10% | 17% | 0.54 | 12% |
| SIEINSSE² | 4% | 42% | 1.69 | 68% |
| HAWENNFG² | 3% | 14% | 1.12 | 47% |
| AFIHEAWS² | 2% | 9% | 1.42 | 46% |
| FWSAFINE² | <1% | 6% | 1.98 | 25% |
| NNWHEWHI² | <1% | 4% | 1.48 | 57% |
| GKEAAFAA³ | 15% | 34% | 1.44 | 55% |
| SKSAAEHE³ | 8% | 32% | 1.24 | 65% |
| SHIKNSAN³ | 8% | 29% | 0.78 | 17% |
| GNSNAAHF³ | 6% | 32% | 0.71 | 7% |
| KKWAIGSK³ | 2% | 7% | 0.94 | 47% |
| SHFAWASE³ | 1% | 5% | 0.91 | 57% |
| IKEIKENN³ | <1% | 6% | 1.71 | 64% |
| NWFWSFNE³ | <1% | 4% | 2.05 | 28% |

TABLE 4
Values of LVV yield measured via p24 ELISA (1, viral particles), qPCR (2,
encapsidated transgenes), and transduction assay in HT1080 cells (3, cell-transducing LVV
units), together with clearance of HEK293 HCPs obtained by purifying LVVs from a HEK293
CCCF (LVV titer ~10¹⁰ vp/mL, corresponding to ~10⁸ TU/mL; HCP titer ~0.3 mg/mL) using
peptide-Poros resins and control Poros ™ 50 HE Heparin and CaptureSelect ™ Lenti VSVG
affinity resins. The purification processes comprised a loading step in 20 mM phosphate
buffer with 75 mM NaCl at pH 6.5, at the RT of either 1 or 3.5 minutes; elution was
conducted in 20 mM citrate buffer with 0.5 M MgCl₂ at pH 6.0.

| | RT: | | | | RT: | | | |
|---|---|---|---|---|---|---|---|---|
| | 3.5 min | | Cell- | | 1 min | | Cell- | |
| | Yield | En- | trans- | | Yield | En- | trans- | |
| | Viral | capsidated | ducing | HCP | Viral | capsidated | ducing | HCP |
| Ligand | Particles¹ | Transgenes² | Units³ | LRV | Particles¹ | Transgenes² | Units³ | LRV |
| EWKAAFIW | 12% | 40% | 22% | 0.84 | 5% | 51% | 15% | 1.33 |
| FEKISNAE | 16% | 44% | 22% | 1.62 | 9% | 69% | 38% | 1.82 |
| GEFENINW | 14% | 51% | 4% | 1.73 | 6% | 13% | 12% | 2.71 |
| GKEAAFAA | 15% | 34% | 17% | 1.44 | 10% | 63% | 41% | 1.87 |
| SIEINSSE | 4% | 42% | 6% | 1.69 | 2% | 84% | 8% | 2.49 |
| SKSAAEHE | 8% | 32% | 25% | 1.24 | 5% | 59% | 29% | 1.75 |
| SNEIEIAN | 23% | 28% | 4% | 1.84 | 12% | 65% | 12% | 2.25 |
| Heparin | 19% | 20% | 18% | 1.44 | 13% | 52% | 39% | 1.79 |
| Capture- | 6% | 38% | 43% | 1.94 | † | | | |
| Select ™ | | | | | | | | |
| Lenti VSVG⁴ | | | | | | | | |

⁴tested according to product instructions: RT of 2 min; equilibration and washing buffer: 50 mM HEPES buffer
with 150 mM NaCl at pH 7.5; elution buffer: 50 mM HEPES buffer with 150 mM NaCl and 0.8 M arginine at pH
7.5; stripping solution: 50 mM sodium phosphate at pH 12.0. †: values not measured.

### Example 3

Optimizing LVV purification by adjusting the composition of the chromatographic buffers The growth of LVV yield and purity obtained simply by reducing the residence time of the loading step suggested that further adjustment of the chromatographic process may further increase the yield and purity. Accordingly, the composition, concentration, and pH of the chromatographic buffers were optimized to improve the performance of FEKISNAE- (SEQ ID NO: 1), GEFENINW- (SEQ ID NO: 7), and GKEAAFAA (SEQ ID NO: 3)-Poros resins. The addition of arginine to the wash buffer and MgCl₂ to the elution buffer was initially explored, and subsequently buffers with different basal composition and conductivity were evaluated, while maintaining a constant RT of 1 minute for the loading step.

As shown in Table 5, the addition 50 mM arginine to the wash buffer (20 mM phosphate buffer with 75 mM NaCl at pH 6.5) increased slightly the HCP LRV obtained with FEKISNAE-Poros (SEQ ID NO: 1) resin from 1.82 to 2.01 (corresponding to a 102-fold reduction and a residual HCP titer of <3 g/mL), suggesting a potential strategy for improve HCP clearance. However, increasing the MgCl₂ concentration from 0.5 M to 1 M in the base elution buffer (20 mM citrate buffer at pH 6.0) reduced LVV recovery and was therefore abandoned.

To explore additional buffer systems with different basal compositions, Tris was first used to formulate new equilibration, wash, and elution buffers. Notably, the new wash buffer (50 mM Tris buffer with 130 mM NaCl at pH 8.0) increased the HCP LRV by FEKISNAE (SEQ ID NO: 1)-Poros resin to 2.39 (corresponding to a 246-fold reduction and a residual HCP titer of 1.2 μg/mL) and that of GKEAAFAA (SEQ ID NO: 3)-Poros resin to 2.05 (112-fold reduction; 2.6 μg/mL). Additionally, the new elution buffer (50 mM Tris and 1 M NaCl at pH 8.0) increased the yield of cell-transducing LVV units afforded by FEKISNAE- (SEQ ID NO: 1) and GKEAAFAA (SEQ ID NO: 3)-Poros respectively to 35% and 38%. Under the same conditions, GEFENINW (SEQ ID NO: 7)-Poros resin provided excellent purify, but unsatisfactory yields; this poor performance, combined with the presence in this peptide of asparagine (N) and tryptophan (W) residues that are prone to degradation, likely due to deamidation to aspartic acid and oxidation, led to abandonment of this candidate ligand.

TABLE 5
Values of LVV recovery measured via p24 ELISA (total particles), qPCR
(total encapsidated transgenes), and transduction assay in HT1080 cells (cell-transducing
LVV units), together with clearance of HEK293 HCPs and DNA obtained by purifying LVVs from
a HEK293 CCCF (LVV titer ~10¹⁰ vp/mL, corresponding to ~10⁸ TU/mL; HCP titer ~0.3 mg/mL)
using FEKISNAE (SEQ ID NO: 1), GEFENINW (SEQ ID NO: 7)-, and GKEAAFAA (SEQ ID
NO: 3)-Poros resins. All purification processes comprised a loading step conducted at the
RT of 1 minute. The values of yield were measured via transduction activity on HT1080 cells.

| | Buffers | | | | HCP |
|---|---|---|---|---|---|
| Ligand | Binding | Wash | Elution | Yield | LRV |
| FEKISNAE | 20 mM | 20 mM phosphate | 20 mM citrate | 8% | 1.81 |
| | phosphate | 75 mM NaCl, pH 6.5 | 0.5 M MgCl₂, pH 6.0 | | |
| | 75 mM NaCl, pH | 20 mM phosphate | 20 mM citrate | 13% | 2.01 |
| | 6.5 | 100 mM NaCl | 0.5 M MgCl₂, pH 6.0 | | |
| | | 50 mM arginine, pH 6.5 | | | |
| | 50 mM Tris | 50 mM Tris | 50 mM Tris | 38% | 2.39 |
| | 130 mM NaCl, | 130 mM NaCl, pH 8.0 | 1 M NaCl, pH 8.0 | | |
| | pH 8.0 | | | | |
| GEFENINW | 20 mM | 20 mM phosphate | 20 mM citrate | 1% | 2.39 |
| | phosphate | 75 mM NaCl, pH 6.5 | 1 M MgCl₂, pH 6.0 | | |
| | 75 mM NaCl, pH | | 20 mM citrate | 1% | 2.37 |
| | 6.5 | | 0.5 M MgCl₂, pH 6.0 | | |
| | 50 mM Tris | 50 mM Tris | 50 mM Tris | <1% | 1.92 |
| | 130 mM NaCl, | 130 mM NaCl, pH 8.0 | 1 M NaCl, pH 8.0 | | |
| | pH 8.0 | | | | |
| GKEAAFAA | 20 mM | 20 mM phosphate | 20 mM citrate | 28% | 1.83 |
| | phosphate | 100 mM NaCl | 0.5 M MgCl₂, pH 6.0 | | |
| | 75 mM NaCl, pH | 50 mM arginine, pH 6.5 | | | |
| | 6.5 | | | | |
| | 50 mM Tris | 50 mM Tris | 50 mM Tris | 35% | 2.05 |
| | 130 mM NaCl, | 130 mM NaCl, pH 8.0 | 1 M NaCl, pH 8.0 | | |
| | pH 8.0 | | | | |

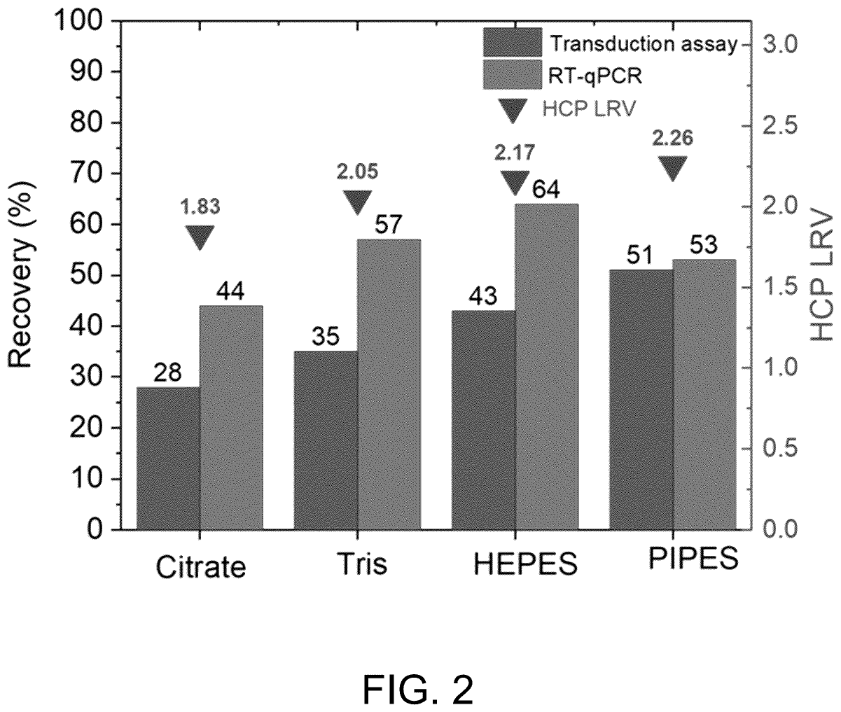

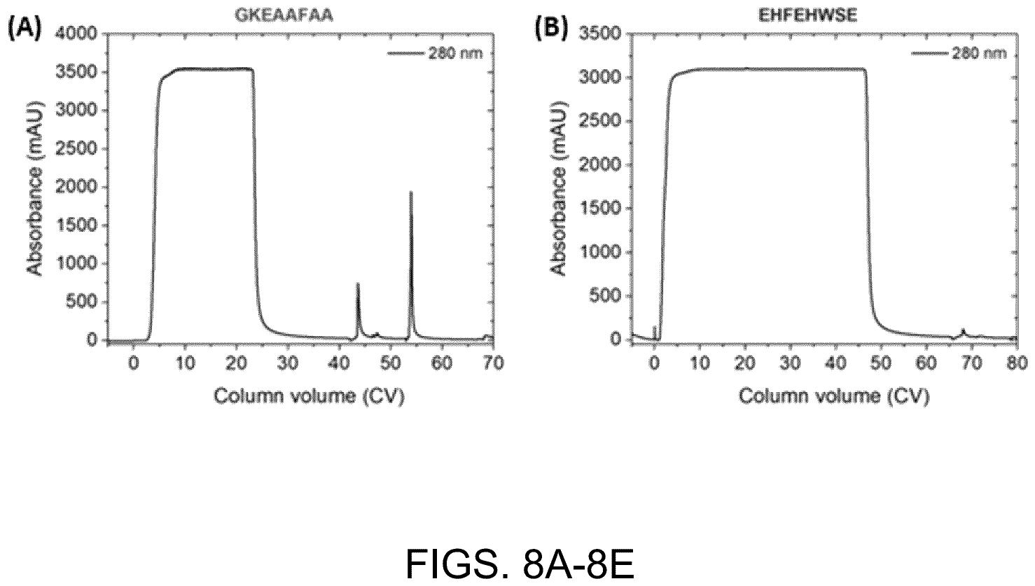

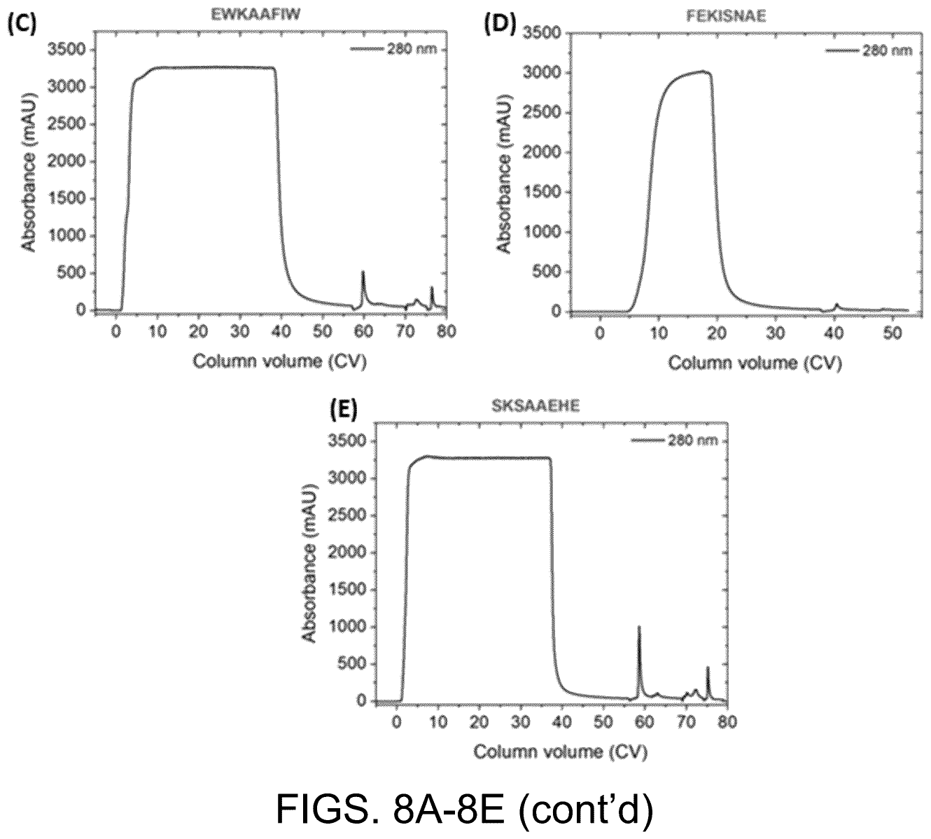

A new set of wash and elution buffers, 50 mM HEPES/PIPES buffer at pH 7.4 added with 100 mM and 0.65 M NaCl, respectively, were next evaluated starting with GKEAAFAA (SEQ ID NO: 3) as the top performing ligand. The results summarized in FIG. 2 show an appreciable improvement in both the yield of cell-transducing LVV units and HCP clearance, which reached respectively a value of 51% and an LRV of 2.26 (i.e., 182-fold reduction; residual titer ˜1.6 g/mL) using PIPES-based buffers. Accordingly, four more candidate sequences, FEKISNAE (SEQ ID NO: 1), and the candidate ligands SKSAAEHE (SEQ ID NO: 6), EWKAAFIW (SEQ ID NO: 8), EHFEHWSE (SEQ ID NO: 12), selected from Table 3 based on their sequence similarity with GKEAAFAA (SEQ ID NO: 3) and FEKISNAE (SEQ ID NO: 1), were reevaluated using the PIPES-based buffers. The resulting chromatograms are in FIG. 8, while the corresponding values of LVV yield and purity are collated in Table 6. Collectively, these results strongly supported the adoption of piperazineethanesulfonate-based buffers, and, more broadly, showcase the impact of chromatographic processing on the transduction activity of the purified LVV particles: while no reduction was observed upon incubation of LVV particles in either HEPES and PIPES buffers, chromatographic processing caused an appreciable loss of infectivity (i.e., purified LVV particles exhibited almost half of the transduction activity of their pre-chromatography counterparts when tested on HT1080 cells at the same encapsidated transgene-to-cell ratio).

TABLE 6
LVV purification using an optimized chromatographic
protocol. Values of LVV recovery measured via p24 ELISA
(total particles) and qPCR (total encapsidated transgenes),
logarithmic removal value of HEK293 host cell proteins
(HCP LRV), and residual double-strand DNA obtained via
chromatographic purification of LVV particles from a
HEK293 CCF (LVV titer ~1010 vp/mL, corresponding to ~108
TU/mL; HCP titer ~0.3 mg/mL) using peptide-Poros resins.
The equilibration and washing steps were conducted using
50 mM PIPES buffer with 100 mM NaCl at pH 7.4 (RT: 1 min);
elution was conducted using 50 mM PIPES buffer with
0.65 M NaCl at pH 7.4 (RT: 1 min).

| | Yield | | | |
|---|---|---|---|---|
| | Viral | Encapsidated | HCP | Residual |
| Ligand | Particles¹ | Transgenes² | LRV | dsDNA |
| SKSAAEHE | 52% | 54% | 1.81 | 23% |
| EWKAAFIW | 17% | 33% | 2.08 | 46% |
| GKEAAFAA | 48% | 74% | 2.07 | 33% |
| EHFEHWSE | 13% | 27% | 2.32 | 11% |
| FEKISNAE | 63% | 55% | 2.39 | 45% |

### Example 4

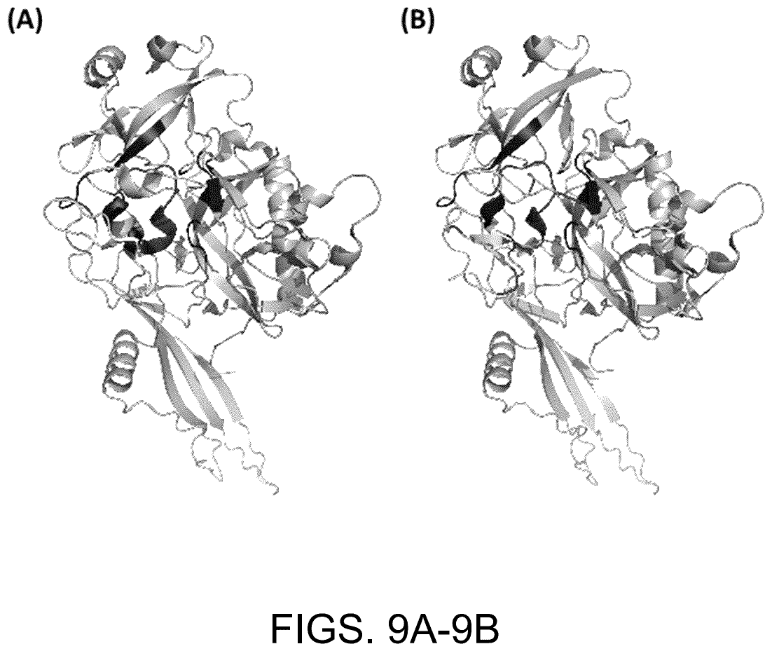

In silico discovery and experimental evaluation of VSV-G-binding peptides. The results presented in the previous sections demonstrate that the peptide sequences identified by screening the peptide library against VSV-G consistently outperformed the sequences selected against full LVV particles. Sequences that target ligand-able sites on VSV-G are presented as a promising route to the discovery of peptide ligands for LVV purification. In this context, the published crystal structure of the complex formed by VSV-G and the cysteine-rich domains of the low-density lipoprotein receptor (LDL-R CR2 and CR3), a cell surface receptor that plays a key role in LVV cell entry, provided insight into the interaction. Two cationic residues on VSV-G, His8 and Lys47, that target anionic residues Asp69 and Asp 73 on CR2 and Asp108 and Asp112 on CR3, provide a major contribution to the LDL-R binding energy. Additionally, the LDL-R CR2 and CR3 domains feature a compact tertiary structure, rigidly held by 3 disulfide bonds, which resembles that of scaffolds (i.e., knottins, avimers, and bicyclic peptides)utilized to discover small protein affinity ligands. Finally, the analysis of pairwise interactions between the active residues on LDL-R CR2 and CR3, reported in FIG. 9 and Table 7, indicated that the VSV-G binding site consists of a continuous segment of residues. Relevantly to this work, these data suggest that (i) small peptide mimetics of LDL-R CR2 and CR3 can be designed in silico to target the same CR-binding site of VSV-G; and (ii) the top-performing ligands identified via library screening, SKSAAEHE (SEQ ID NO: 6), GKEAAFAA (SEQ ID NO: 3), and FEKISNAE (SEQ ID NO: 1), all of which contain at least one glutamic acid residue, may indeed target the LDL-R CR-binding site of VSV-G.

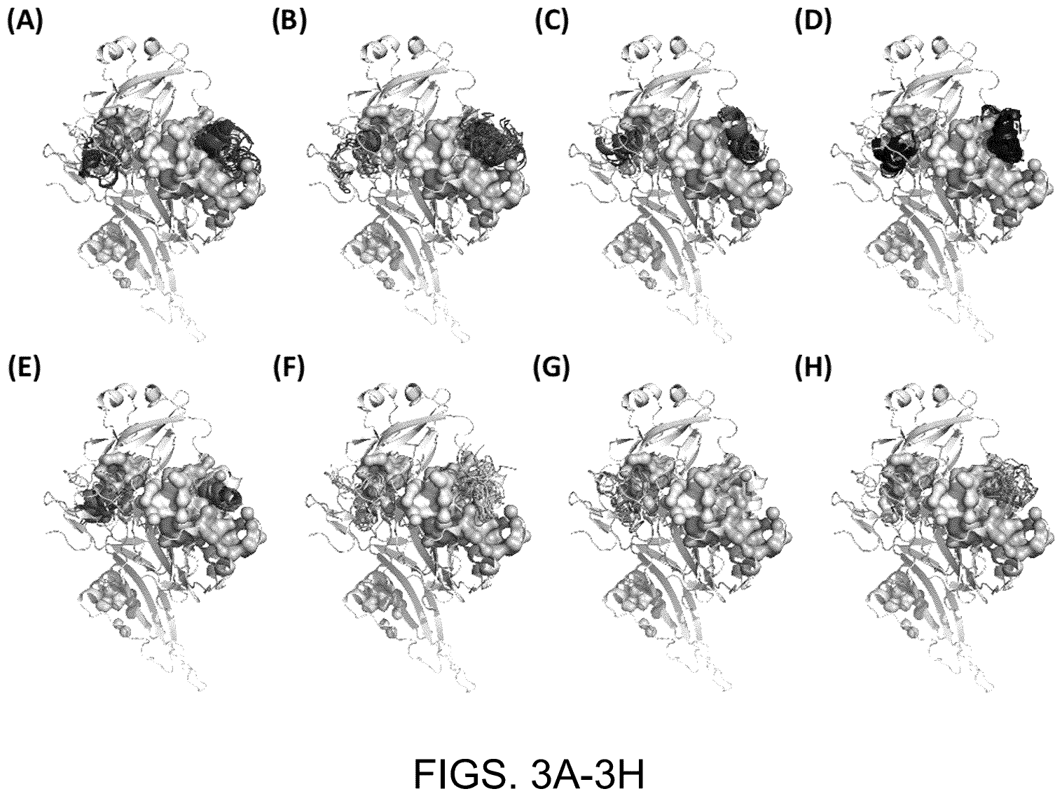

Accordingly, an in silico ensemble of candidate ligands were designed, whose sequence and structure mimic the LDL-R CR2 and CR3 domains: specifically, the four disulfide-cyclic sequences C-cyclo[GSRQFVADSDRD]C-GSG (SEQ ID NO: 87), C-cyclo[GSRSFVGDSDRD]C-GSG (SEQ ID NO: 88), C-cyclo[GSRAFVADADRD]C-GSG (SEQ ID NO: 98), C-cyclo[GSRAFVGDAD]C-GSG (SEQ ID NO: 99), and the five linear sequences SRQFVCGDSDRD-GSG (SEQ ID NO: 100), SRSFVCDSDRD-GSG (SEQ ID NO: 101), SRAFVGDADRD-GSG (SEQ ID NO: 102), AFVGDADRD-GSG (SEQ ID NO: 103), and SFVRIGLSD-GSG (SEQ ID NO: 104). The sequence homology and the small values of root-mean-square deviation (RMSD) of the atomic positions of the designed peptides vs. their cognate CR2 and CR3 domains provide confidence in the LDL-R-mimetic behavior of the proposed sequences. The eight designed peptides, along with SKSAAEHE (SEQ ID NO: 6), GKEAAFAA (SEQ ID NO: 3), FEKISNAE (SEQ ID NO: 1), and the latter's variants FEKISAAE (SEQ ID NO: 2) and FEKISTAE (SEQ ID NO: 11), were docked in silico against the crystal structures of VSV-G (PDB IDs: 5OY9 and 5OYL) in different aqueous environments that represent the various buffers utilized during the purification process, namely ionic strength and pH of 150 mM and 7.4 to represent the binding buffer, and 0.7 M and pH 7.4 or 1 M and pH 6.0 representing two alternative elution buffers (namely, 50 mM PIPES buffer with 0.65 M NaCl at pH 7.4; 20 mM citrate with 0.5 M MgCl2, pH 6.0). Peptide docking was focused on the putative binding sites identified on the solvent-accessible surface of the protein as “ligandable,” namely whose physicochemical and topological characteristics make it apt to bind a biomolecular ligand with true affinity. The other key constraint imposed during docking is for the -GSG tripeptide appended on the C-terminal end not to interact with the target VSV-G: this forces the -GSG tripeptide to orient outward from the binding pose, thus mimicking the orientational constraint imposed to the peptides by their conjugation on the surface of the chromatographic resin. In prior studies, this constraint has delivered superior accuracy in estimating the target binding energy. The resultant VSV-G:peptide complexes, selected based on their cluster size and initial scoring using X-Score, were subjected to 250-ns MD simulations in explicit-solvent conditions that represent the binding and elution buffers to obtain reliable values of binding free energy (ΔG_{b}). Selected complexes are shown in FIG. 3 and the corresponding values of dissociation constant (K_{D,in silico}) are listed in Table 8.

TABLE 7
Pairwise interactions between the active residues
on VSV-G and the targeted residues on LDL-R
CR2 (PDB ID: 5OYL) and CR3 (5OY9) domains.

| LDL-R | ΔGb | | LDL-R | ΔGb | |
|---|---|---|---|---|---|
| CR3 | (kcal/mol) | VSV-G | CR2 | (kcal/mol) | VSV-G |
| GLN90 | −0.101 | MET184 | GLY40 | −0.66 | ASP178 |
| ASP91 | −0.202 | LYS47 | | | SER179 |
| LYS99 | −0.101 | ASN9 | | | ASN180 |
| SER102 | −0.808 | LYS11 | PRO41 | −0.33 | SER179 |
| ARG103 | −0.707 | TYR209 | | | ASN180 |
| | | ARG354 | LEU42 | −1.1 | SER48 |
| | | GLU355 | | | SER179 |
| | | ILE52 | | | ILE182 |
| GLN104 | −0.909 | ARG354 | | | ASN180 |
| | | ILE347 | | | LYS47 |
| | | THR352 | PRO63 | −0.44 | ALA51 |
| | | LYS47 | GLN64 | −0.22 | GLN10 |
| | | ILE331 | PHE65 | −0.88 | GLN10 |
| | | ALA51 | | | ASN9 |
| | | ILE182 | | | ARG354 |
| PHE105 | −1.010 | THR350 | | | ALA51 |
| | | GLN10 | | | HIS8 |
| | | SER48 | TRP66 | −1.1 | LYS47 |
| | | ILE347 | | | GLN10 |
| | | LYS47 | | | ARG354 |
| VAL106 | −0.303 | ARG354 | | | ALA51 |
| | | SER179 | | | SER48 |
| CYS107 | −0.909 | ILE182 | | | HIS8 |
| | | SER183 | ARG67 | −0.22 | GLN10 |
| | | HIS8 | | | ARG354 |
| | | ASN9 | CYS68 | −0.99 | ARG354 |
| ASP108 | −1.010 | THR352 | | | THR352 |
| | | ARG354 | | | GLU353 |
| | | ARG342 | | | ILE347 |
| | | ALA51 | ASP69 | −1.1 | ILE331 |
| | | GLN53 | | | ILE347 |
| | | MET184 | | | ARG354 |
| | | TYR209 | | | TYR209 |
| SER109 | −0.606 | TYR209 | | | HIS8 |
| | | SER183 | | | THR352 |
| | | HIS8 | | | LYS47 |
| | | GLU353 | GLY70 | −0.77 | ILE347 |
| | | LYS11 | | | TYR209 |
| | | VAL344 | | | SER183 |
| ASP110 | −1.010 | HIS8 | | | MET184 |
| | | GLN10 | | | THR352 |
| | | THR352 | | | ASP185 |
| | | ILE182 | GLN71 | −1.1 | LYS47 |
| | | ARG354 | | | TYR209 |
| ARG111 | −0.202 | ALA51 | | | ILE182 |
| | | ILE347 | | | HIS8 |
| ASP112 | −0.808 | ARG354 | | | SER183 |
| | | THR352 | | | MET184 |
| GLU119 | −0.202 | ARG354 | VAL72 | −0.55 | ILE182 |
| CYS122 | −0.404 | HIS8 | | | SER183 |
| | | THR352 | | | MET184 |
| | | GLU353 | ASP73 | −0.88 | ILE182 |
| PRO123 | −0.505 | GLN10 | | | LYS47 |
| | | LYS11 | ASP75 | −0.11 | ASN180 |
| | | SER183 | GLU80 | −0.33 | THR352 |
| | | LYS47 | CYS83 | −0.88 | THR351 |
| | | GLU353 | | | GLU353 |
| Total | −9.8 | | | | THR352 |
| | | | PRO84 | −0.44 | THR351 |
| | | | | | ARG342 |
| | | | | | GLU353 |
| | | | Total | −12.1 | |

TABLE 8
Values of dissociation constant (K_{D,in silico}) of the complexes formed by LDL-
R-mimetic peptides designed in silico and the peptides identified via library screening with
VSV-G obtained via molecular docking and dynamics simulations in explicit solvent conditions
that mimic the binding (ionic strength of 150 mM and pH of 7.4) and elution (A: 0.7 M and
pH 7.4; B: 1 M and pH 6.0) buffers.

| | CR2/3 vs. | Site 1 (LDL-R binding site) | | | Site 2 | | |
|---|---|---|---|---|---|---|---|
| | peptide | Binding | Elution A | Elution B | Binding | Elution A | Elution B |
| Ligand | RMSD (Å) | (M) | (M) | (M) | (M) | (M) | (M) |
| C- | 2.37 Å | 1.05·10⁻⁶ | 6.57·10-⁵ | 4.64·10-⁵ | 5.05·10-⁷ | 6.50·10-⁵ | 9.66·10-⁵ |
| cyclo[GSRQFVADSD | | | | | | | |
| RD]C-GSG | | | | | | | |
| C- | 2.56 Å | 1.00·10-⁶ | 6.68·10-⁵ | 4.77·10-⁵ | 6.05·10-⁷ | 6.82·10-⁵ | 1.07·10-⁴ |
| cyclo[GSRSFVGDSDR | | | | | | | |
| D]C-GSG | | | | | | | |
| C- | 2.47 Å | 1.38·10-⁴ | 1.47·10⁻⁴ | 1.05·10-⁴ | 2.13·10-⁴ | 1.40·10-⁴ | 2.10·10-⁴ |
| cyclo[GSRAFVADAD | | | | | | | |
| RD]C-GSG | | | | | | | |
| C- | 2.42 Å | 1.08·10-⁶ | 1.68·10⁻⁴ | 1.07·10-⁴ | 5.00·10-⁷ | 1.65·10-⁴ | 2.40·10-⁴ |
| cyclo[GSRAFVGDAD] | | | | | | | |
| C-GSG | | | | | | | |
| SRQFVCGDSDRD- | 1.95 Å | 1.05·10-⁶ | 1.58·10⁻⁴ | 1.16·10-⁴ | 5.00·10-⁷ | 1.41·10-⁴ | 2.29·10-⁴ |
| GSG | | | | | | | |
| SRSFVCDSDRD-GSG | 1.87 Å | 1.03·10-⁵ | 5.60·10-⁵ | 4.08·10-⁵ | 5.05·10-⁵ | 5.71·10-⁵ | 8.00·10-⁵ |
| SRAFVGDADRD- | 1.81 Å | 1.36·10-⁶ | 8.53·10⁻⁴ | 7.35·10-⁴ | 6.75·10-⁷ | 8.75·10-⁴ | 1.11·10-³ |
| GSG | | | | | | | |
| AFVGDADRD-GSG | 1.76 Å | 1.01·10-⁴ | 1.40·10⁻⁴ | 1.01·10-⁴ | 1.06·10-⁴ | 1.41·10-⁴ | 2.02·10-⁴ |
| SFVRIGLSD-GSG | 1.58 Å | 1.12·10-⁶ | 8.33·10⁻⁴ | 5.30·10-⁴ | 5.50·10-⁷ | 8.53·10-⁴ | 1.05·10-³ |
| FEKISNAE | † | 1.93·10-⁶ | 3.78·10⁻⁴ | 1.99·10-⁴ | 1.07·10-⁶ | 4.41·10-⁴ | 6.36·10-⁴ |
| FEKISAAE | | 2.74·10-⁶ | 1.84·10⁻⁴ | 2.31·10-⁴ | 1.38·10-⁶ | 1.91·10-⁴ | 2.73·10-⁴ |
| FEKISTAE | | 1.41·10-⁶ | 5.25·10⁻⁴ | 1.23·10-⁴ | 9.05·10-⁷ | 5.29·10-⁴ | 9.15·10-⁴ |
| GKEAAFAA | | 3.70·10-⁶ | 1.46·10⁻⁴ | 3.83·10-⁴ | 1.65·10-⁶ | 1.50·10-⁴ | 2.14·10-⁴ |
| SKSAAEHE | | 1.79·10-⁶ | 5.95·10⁻⁴ | 1.52·10-⁴ | 9.70·10-⁷ | 4.75·10-⁴ | 7.55·10-⁴ |
| LDL-R CR2 (50YL) | — | 1.41·10-⁷ | 6.75·10-⁶ | 9.82·10-⁶ | § | | |
| LDL-R CR2 (50Y9) | | 6.92·10-⁸ | 5.01·10-⁶ | 3.73·10-⁶ | | | |

†peptides GKEAAFAA, SKSAAEHE, and FEKISNAE and its derivatives FEKISAAE and FEKISTAE were not desired as LDL-R mimetics.
§this site is not targeted by the CR2 and CR3 domains of LDL-R.

The results of molecular docking support the design criteria of the mimetic sequences: (i) all peptides, with the sole exception of SKSAAEHE (SEQ ID NO: 6), formed complexes whose binding pairwise interactions recapitulate those of the VSV-G:LDL-R complexes; (ii) the binding strength of the VSV-G:peptide complex in the binding environment is moderately lower (5.9-8.7 kcal/mol; K_{D}˜5·10⁻⁷-5·10⁻⁶ M) than that of their VSV-G:LDL-R CR2 and CR3 precursors (9.3-9.7 kcal/mol; K_{D}˜5·10⁻⁸-10⁻⁷ M); and (iii) all peptides except SRTFVCDSDRD (SEQ ID NO: 94) exhibited comparable affinity for a second binding site (described by the green surface in FIG. 3). The ability of peptide ligands to target multiple binding sites on the target surface with true affinity suggested the formation of a multi-site interaction network between the virus and the peptide-functionalized surface. The LVV coat displays approximately 216 VSV-G proteins, suggesting that VSV-G are placed at 12.5-31.3 Å from each other (assuming the LVV radius to be 40-50 nm); additionally, based on the peptide density on the resin (˜30 mol per gram) and the resin's specific surface (˜30 m²/g), the peptides are displayed at ˜18 Å from each other. This suggests the formation of 8-10 VSV-G:peptide interactions per bound LVV particle. The cooperation of multiple affinity interactions results in a strong avidity-like binding that promotes efficient and selective LVV capture, as demonstrated by the values of binding capacity and product purity presented below.

Furthermore, as shown in Table 8, the dissociation constant (K_{D}) of the VSV-G:peptide complexes undergoes a 540-to-750-fold increase as the ionic strength of the environment increases from 150 mM to 1.3 M (representing the 50 mM PIPES elution buffer containing 0.65 M MgCl₂) and a 1,550-to-1,900-fold increase as the pH decreases from 7.4 to 6.0 (representing the 20 mM citrate elution buffer). This suggested that the adsorbed viruses can be effectively released under conditions that safeguard their transduction activity, as confirmed by the values of product yield. The analysis of the molecular simulation trajectories indicated that the VSV-G:peptide dissociation is strongly influenced by the loss of (i) Coulombic interactions between the anionic residues in the peptide ligands and their cationic counterparts on the VSV-G, chiefly Lys47, Arg342, and Arg354, which contribute ˜34-41% of the binding energy at pH 7.4.

The second major contributor to the free energy of binding, the network of hydrogen bonds and polar interactions, e.g., those formed with Gln10, Ser179, Asn180, Ser183, Thr350-352, and Glu353, which contribute 31-39% of the binding energy, was also obliterated by the addition of MgCl₂, a known chaotrope which destabilizes the electrostatic and hydrogen bonding interactions. The increase of ionic strength and the decrease of pH also causes a contraction of the VSV-G solvent accessible pockets: albeit small, this rearrangement significantly reduces the structural complementarity of the putative pockets to the peptide ligands and promotes their dislodging from the coat proteins. The energy of both VSV-G:LDL-R CR2 and CR3 complexes is also reduced when the simulation environment is switched to elution conditions. However, their residual strength at the reference elution conditions (ΔG_{b}˜7.2-8.2 kcal/mol) is higher than what observed among the VSV-G:peptide complexes, suggesting that product elution from protein ligands is more challenging; this could explain why stronger denaturing conditions are required for lentivirus elution from antibody-based ligands (e.g., 0.8 M arginine is recommended for elution from CaptureSelect™ Lenti VSVG affinity resin).

Based on the predicted values of VSV-G affinity at pH 7.4 and loss of binding upon application of elution conditions, peptides C-cyclo[GSRAFVGDAD]C (SEQ ID NO: 16), SRQFVCGDSDRD (SEQ ID NO: 17), SRAFVGDADRD (SEQ ID NO: 4), and SFVRIGLSD (SEQ ID NO: 5) were conjugated on Poros resins and evaluated by purifying LVVs from a clarified HEK293 cell culture harvest using the optimized PIPES-based buffer system. The results summarized in Table 9 confirm the criteria adopted in the in silico peptide design: the cyclic peptide afforded the highest value of HCP clearance registered in this study (i.e., residual HCP titer˜0.34 g/mL corresponding to an 871-fold reduction), but returned a rather unsatisfactory amount of product. Conversely, the SRAFVGDADRD (SEQ ID NO: 4) and SFVRIGLSD (SEQ ID NO: 5) afforded a yield of cell-transducing LVV units comparable to those obtained with FEKISNAE (SEQ ID NO: 1) and GKEAAFAA (SEQ ID NO: 3), while still providing >100-fold reduction of HCPs and ˜68-fold reduction of DNA, and were therefore selected for further characterization.

TABLE 9
LVV purification using LDL-R-mimetic peptides designed in silico. Values
of yield (1: encapsidated transgenes measured via qPCR; 2: cell-transducing LVV
units measured via flow cytometry), logarithmic removal value of HEK293 host
cell proteins (HCP LRV), and residual double-strand DNA obtained via
chromatographic purification of LVV particles in bind-and-elute mode from a
HEK293 CCCF (LVV titer ~10¹⁰ vp/mL, corresponding to ~10⁸ TU/mL; HCP titer
~0.3 mg/mL) using LDL-R-mimetic peptide-Poros resins. The equilibration and
washing steps were conducted using 50 mM PIPES buffer with 100 mM NaCl at pH
7.4 (RT: 1 min); elution was conducted using 50 mM PIPES buffer with 0.65 M
NaCl at pH 7.4 (RT: 1 min).

| | Yield | Cell-transducing | | |
|---|---|---|---|---|
| | Encapsidated | Viral | HCP | Residual |
| Ligand | Transgenes¹ | Particles² | LRV | dsDNA |
| C-cyclo[GSRAFVGDAD]C-GSG | 16% | 12% | 2.94 | 8 |
| SRQFVCGDSDRD-GSG | 18% | 15% | 2.42 | 25 |
| SRAFVGDADRD-GSG | 60% | 45% | 2.20 | 32 |
| SFVRIGLSD-GSG | 55% | 38% | 2.02 | 26 |

These results support the development—in silico or in vitro—of VSV-G-targeting peptides as affinity ligands for the purification of lentivirus from recombinant feedstocks. Owing to their moderate affinity and ability to form multiple interactions leading to strong avidity-driven product capture, VSV-G-targeting peptides can match antibody-based ligands in terms of binding capacity and selectivity, while outperforming them in product yield under non-denaturing conditions. Additionally, the adoption of chemically stable amino acids in constructing the resin-bound library or the in silico ensemble of LDL-R mimetics, and the lack of tertiary structure characteristic of short peptides are conducive to the selection of ligands that are likely more robust than protein binders. The latter aspect is particularly relevant in biopharmaceutical manufacturing, as it impacts the number of uses that an affinity resin can withstand, which represents a key determinant of operational costs of a process—and ultimately the price of the drug to patients.

### Example 5

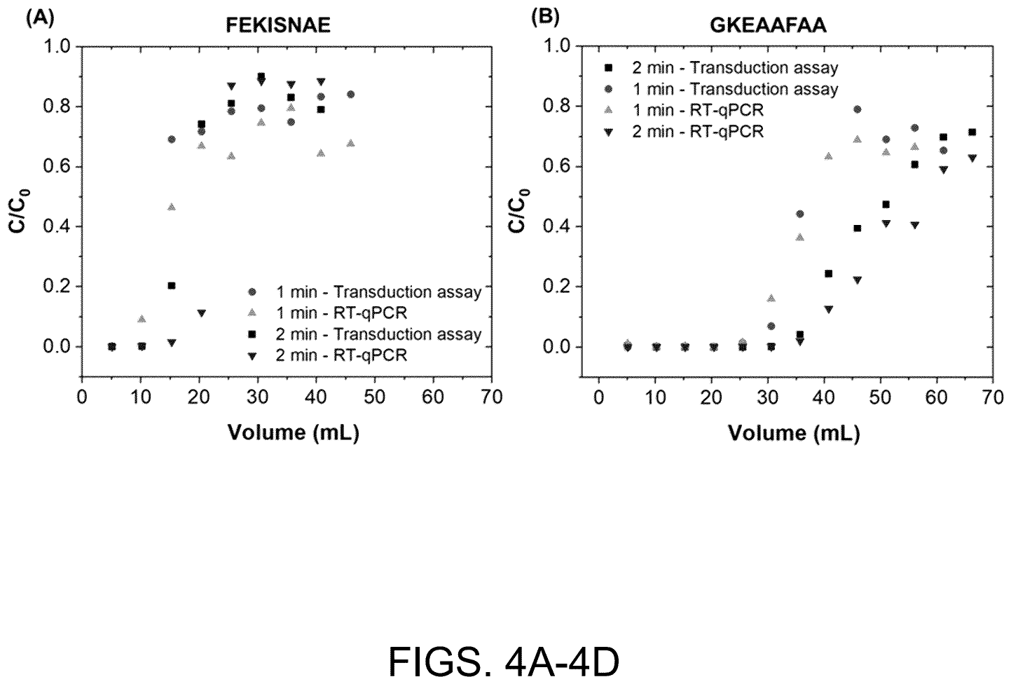

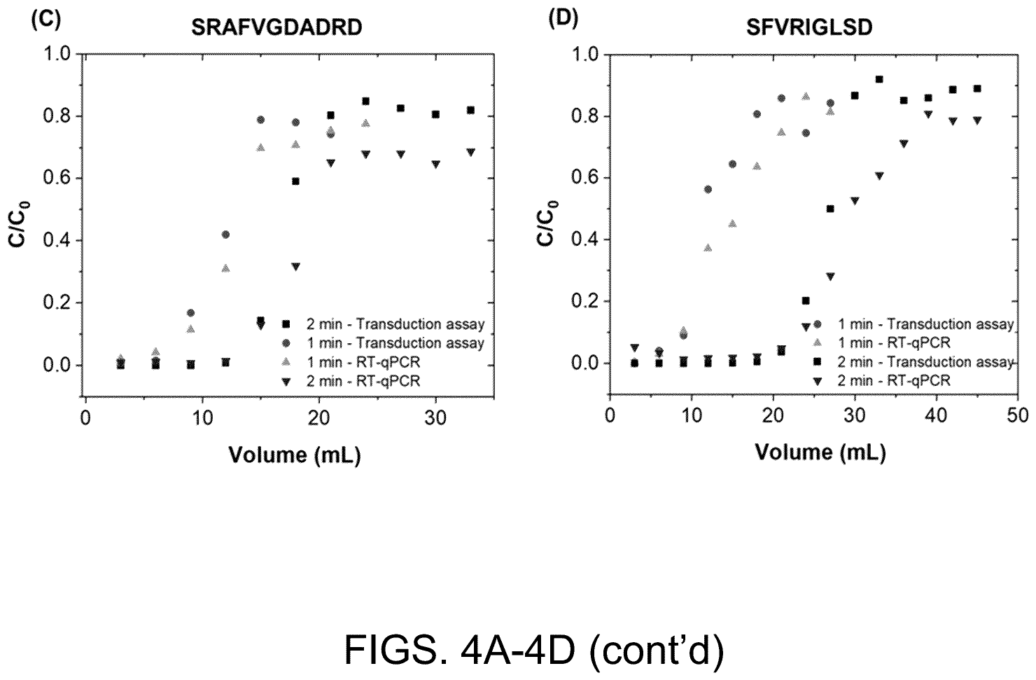

Dynamic binding capacity and alkaline stability of peptide-Poros resins. Based on the results in Tables 6 and 9, the dynamic binding capacity (DBC_{10%}) and stability of adsorbents FEKISNAE- (SEQ ID NO: 1), GKEAAFAA (SEQ ID NO: 3)-, SRAFVGDADRD (SEQ ID NO: 4)-, and SFVRIGLSD (SEQ ID NO: 5)-Poros resins was measured. Unlike the conventional literature, where the values of DBC_{10%} are measured by loading solutions of pure virus and are therefore not representative of realistic process streams, the breakthrough experiments were conducted by loading a clarified bioreactor harvest containing LVV particles at a titer of ˜10⁸ TU/mL. The measurements were conducted at two values of residence time, 2 and 1 min: the former is recommended for CaptureSelect™ Lenti VSVG affinity resin and was adopted in this work for comparability; the latter was adopted to reduce the process time of LVV particles and achieve a higher yield of cell-transducing LVV units. The results reported in FIG. 4 and the corresponding values of DBC_{10%} summarized in Table 10 demonstrate that all peptide-based adsorbents possess high DBC_{10%}, on par with or above the values of commercial affinity resins. Specifically, GKEAAFAA (SEQ ID NO: 3)-Poros and SFVRIGLSD (SEQ ID NO: 5)-Poros resins featured a remarkable DBC_{10%} of 1.91·10¹⁰ and 3.99·10¹⁰ vg/mL at the RT of 1 min; FEKISNAE (SEQ ID NO: 1)-Poros and SRAFVGDADRD (SEQ ID NO: 4)-Poros resins showed slightly lower, yet still appreciable, values of DBC_{10%} values of 5.84·10⁹ and 6.89·10⁹ vg/mL (RT of 1 min). For reference, the DBC_{10%} of CaptureSelect™ Lenti VSVG resin is 9.73·10¹⁰ vp/mL at the RT of 2 min, while that of Poros™ 50 HE Heparin affinity resin is 1.0·10⁸ TU/mL at the RT of 0.5 min. The ability of peptide-functionalized adsorbents to capture a comparable amount of LVV particles while reducing the process time of 50% may stem from the “flexible” biorecognition mechanism of peptide ligands. As suggested by the docking studies, which returned several high-probability binding poses for each sequence (FIG. 3), the interactions with VSV-G formed by peptide ligands appear to be less orientation-dependent than those formed by proteins. This may promote the rapid formation of multi-site binding of LVV particles by the peptide-functionalized surface, manifested in the form of faster adsorption kinetics, which translates in equal binding capacity at lower residence time or higher capacity at a longer residence time.

The ratio of LVV titer in the effluent (C) did not reach the corresponding value in the load (C₀) at plateau. Segura et al. and Moreira et al. also reported a C/C₀ plateau˜0.8 when measuring the LVV binding capacity of heparin-functionalized resins (See, Biotechnol. Bioeng., 2005, 90: 391-404 and Int. J. Mol. Sci. 2023, 24, 3354, respectively). To assess the role of LVV loss in the tubing on the plateau value of the LVV titer, HEK293 CCCF was loaded on the FPLC system without a column and a transduction assay of the effluent fractions was conducted as soon as they were dispensed on fraction collector. As anticipated, the analysis of the effluents showed a 5-10% loss in LVV transduction activity, which can be ascribed to shear, non-ideal temperature, or adsorption on the inner walls of the chromatographic equipment (note: FPLC system is constructed with inert tubes).

TABLE 10
Values of dynamic LVV binding capacity (DBC10%) of peptide-Poros
resins loaded with HEK293 CCCF (LVV titer ~10¹⁰ vp/mL, corres-
ponding to ~10⁸ TU/mL; HCP titer ~0.3 mg/mL) at the residence
time of either 1 or 2 min. The LVV titer in the effluent was
measured via real time qPCR (vg/mL) and transduction assay (TU/mL).

| | RT | DBC_{10%} | |
|---|---|---|---|
| Ligand | (min) | (vg/mL) | (TU/mL) |
| FEKISNAE | 1 | 7.03 10⁹ | 1.43 10⁹ |
| | 2 | 5.84 10⁹ | 1.69 10⁹ |
| GKEAAFAA | 1 | 1.91 10¹⁰ | 4.04 10⁹ |
| | 2 | 2.69 10¹⁰ | 5.24 10⁹ |
| SRAFVGDADRD | 1 | 6.89 10⁹ | 1.31 10⁹ |
| | 2 | 9.63 10⁹ | 2.30 10⁹ |
| SFVRIGLSD | 1 | 3.99 10¹⁰ | 1.41 10⁹ |
| | 2 | 8.07 10¹⁰ | 3.76 10⁹ |
| CaptureSelect ™ Lenti | 2 | 9.73 10⁹ vp/mL | |
| VSVG | | | |

| | | |
|---|---|---|
| Heparin | 0.5 | 1.0· 10⁸ TU/mL |

Together with binding capacity, another critical parameter in downstream bioprocessing is the resin stability to cleaning-in-place (CIP). The caustic treatments with concentrated aqueous sodium hydroxide (0.1-0.5 M) established in antibody manufacturing are now being transferred to the production of viral vectors for in vivo and ex vivo gene therapy. Commercial resins POROS CaptureSelect AAVX and AVIPure affinity resins for AAV purification are designed to withstand multiple cycles of reuse with intermediate caustic cleaning. At present, however, these ligands have not yet reached the chemical stability of latest-generation Protein A for mAb purification, whose decades of engineering has made it capable of withstanding many cycles of cleaning with 0.5 M NaOH. Similarly, the affinity technology for LVV purification is still in its infancy, and the newly introduced ligands have not yet accessed the molecular engineering pathway leading to high chemical stability; accordingly, the recommended CIP conditions for Poros™ 50 HE Heparin and CaptureSelect™ Lenti VSVG resins are limited to 25 mM NaOH.

The lability of protein-based ligands has been often linked to the deamidation of asparagine/glutamine (N/Q) residues, as observed in native Protein A, and the loss of tertiary structure caused by the exposure to highly pH. Conversely, 3 of the 4 selected peptides—namely, GKEAAFAA (SEQ ID NO: 3), SRAFVGDADRD (SEQ ID NO: 4), and SFVRIGLSD (SEQ ID NO: 5)—do not contain either N or Q and they only feature a secondary α-helical structure, which can be rapidly recovered upon incubation in neutral pH. On the other hand, FEKISNAE (SEQ ID NO: 1) is expected to convert to FEKISDAE when subjected to alkaline cleaning, due to the deamidation of N to aspartic acid (D). When exposed to a flow of 50 mM NaOH, in fact, FEKISNAE (SEQ ID NO: 1)-Poros resin lost ˜50% of its binding capacity and, following a static contact with 0.1 M NaOH for 30 minutes, did not show any measurable binding of LVV particles. Therefore, alkaline-stable variants FEKISAAE (SEQ ID NO: 2) and FEKISTAE (SEQ ID NO: 11) were designed in silico to possess VSV-G binding and elution activity comparable to those of the cognate sequence (Table 8).

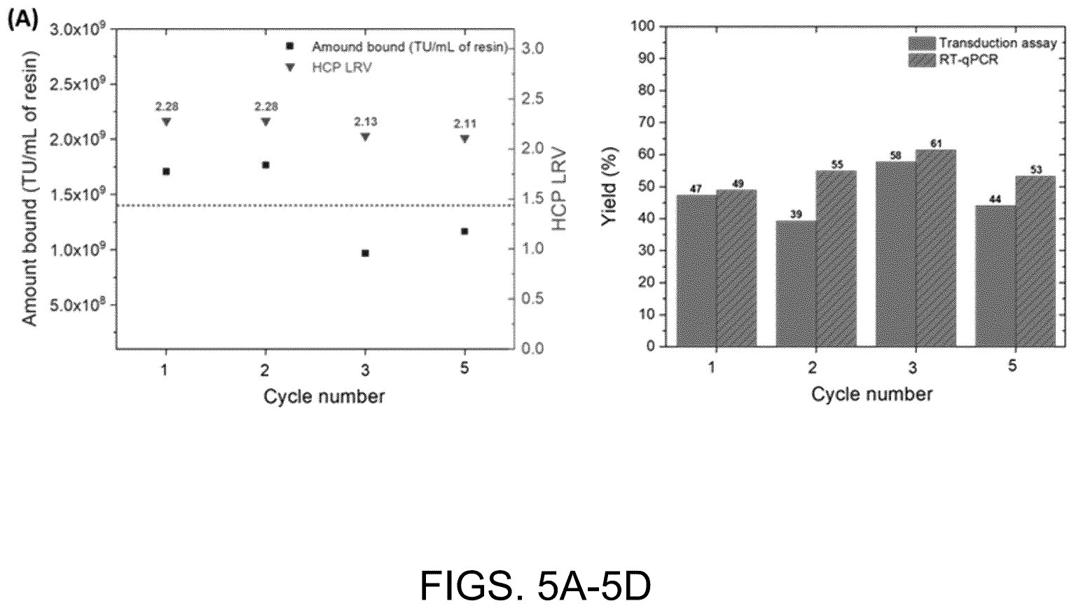

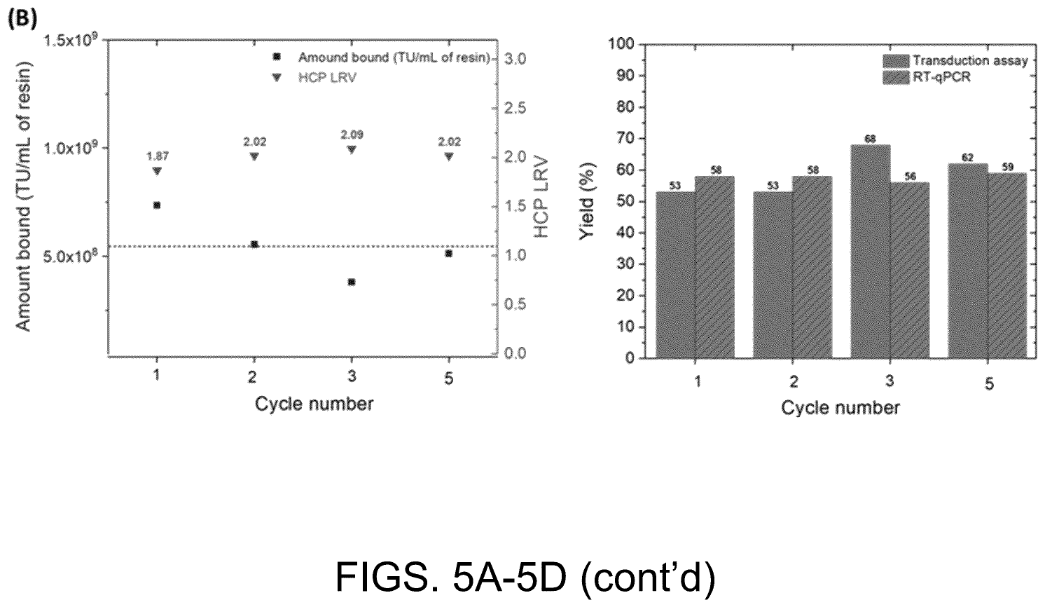

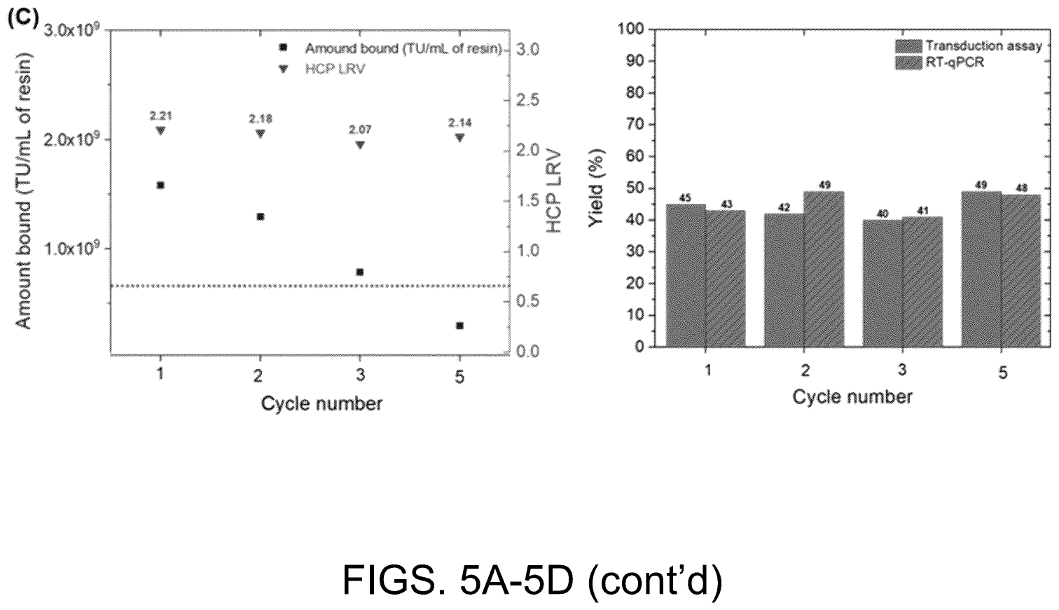

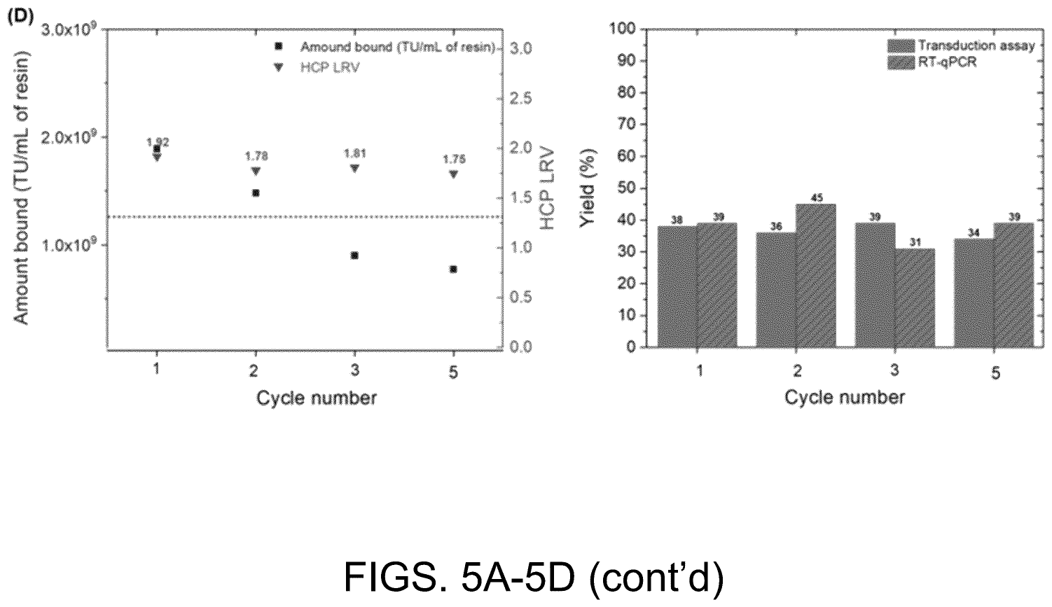

Adsorbents GKEAAFAA (SEQ ID NO: 3)-, FEKISAAE- (SEQ ID NO: 2), SRAFVGDADRD (SEQ ID NO: 4)-, and SFVRIGLSD (SEQ ID NO: 5)-Poros resins were subjected to five consecutive cycles of LVV purification from the HEK293 CCF with intermediate CIP with 0.5 M NaOH. The lifetime study presented in FIG. 5 corroborates the criteria adopted in peptide design. Specifically, GKEAAFAA (SEQ ID NO: 3) maintained its binding capacity and selectivity, consistently adsorbing >10⁹ TU/mL and affording an average yield ≥40% of cell-transducing LVV units (≥50% by qPCR) and a 130-300-fold reduction of HEK293 HCPs (FIG. 5A). FEKISAAE (SEQ ID NO: 2) demonstrated a purification performance on par with its cognate FEKISNAE (SEQ ID NO: 1), while possessing a significantly higher stability: throughout the 5 subsequent purification cycles, the adsorbent maintained its capacity (˜5 10⁸ TU/mL) and afforded a product yield consistently above 50% together with a >200-fold reduction of HCPs (FIG. 5B). Conversely, FEKISTAE (SEQ ID NO: 11)-Poros resins displayed significant reduction of LVV bound to column within increase on number of CIP cycles (data not shown); the slight acidity of the threonine residue may display a negative charge that lowers the binding capacity of this peptide. Finally, SRAFVGDADRD (SEQ ID NO: 4) and SFVRIGLSD (SEQ ID NO: 5) maintained high LVV binding and elution yield (˜10⁹ TU/mL, >38%, respectively) as well as impurity clearance across the consecutive cycles.

### Example 6

Lentivirus purification performance of chromatographic resins functionalized with an affinity peptide ligand targeting VSV-G. In the next series of examples, experiments were conducted to characterize the peptides by evaluating their performance on different matrices, focusing on binding capacity, recovery of cell-transducing LVV units, and removal of host cell proteins (HCPs). To that end, resins of different material composition, particle and pore diameter, and functional density were tested. Experiments were also conducted to test membranes with different fiber morphology, porosity, and ligand distribution.

The material composition, bead diameter, pore size distribution, specific surface area, and ligand density are critical design parameters that determine the binding capacity and selectivity of chromatographic substrates, and therefore the throughput and quality of the purified products. Most of the published literature in this field focuses on the isolation of therapeutic proteins, especially monoclonal antibodies using affinity (e.g., Protein A, Protein G, and Protein L) mixed-mode, and ion-exchange ligands. Yet, the impact of the above-listed parameters on the productivity and quality is arguably more pronounced with viral vectors, due to their larger size and complexity and lower stability. At the same time, this dependence is rather arduous to predict and must be ascertained experimentally. Several studies investigated these phenomena in the context of the purification of adeno-associated viral vectors (AAVs), focusing on either affinity adsorbents for product capture or anion-exchange for product polishing. Analogous studies on lentiviral vectors (LVVs) are, on the other hand, much less abundant, despite their growing relevance in cell and gene therapies. This is due partly to the difficult handling and analysis of LVVs and partly to the very recent introduction of affinity ligands targeting LVVs pseudo-typed with VSV-G proteins. Seeking to fill this knowledge gap, this study focuses on the purification of LVVs using chromatographic substrates of different composition and morphology, beginning with resins made of polystyrene (PS, Poros), poly(methyl methacrylate) (PMMA, ToyoPearl®), poly(vinylether) (PVE, Eshmuno), polyacrylamide/azlactone (Ultralink), or crosslinked agarose (WorkBeads). The pore size of these matrices encompasses a broad range (50-10000 nm), while bead diameter ranges from 45 to 75 μm.

The highest values of DBC_{10%} (1.9-5.0·10⁹ TU per mL of resin) were obtained using Eshmuno, ToyoPearl® and Poros™ resins, likely owing to their higher functionalization, which translates in a higher ligand density (0.05-0.2 mmol of peptide mL of resin, mmol/ /mL); in contrast, the capacity of agarose- and polyacrylamide/azlactone-based resins, whose ligand density is more modest (0.02-0.05 mmol/mL), was consistently below 10⁹ TU/mL (Table 11). The recovery of bound LVV particles appears to correlate to the pore diameter: Poros, ToyoPearl® 750, Eshmuno 800-50, and GenScript agarose resins, which feature larger pores returned between 60-70% of fed LVVs, whereas the other resins afforded lower product yield.

Based on the values of recovery, the values of Productivity, a key process-relevant parameter defined as the number of cell-cell-transducing LVV units purified by 1 mL of resin in 1 min (TU/mL-min) were derived. The values of varied widely, ranging from as little as 2.4·10⁸ TU/mL·min of GKEAAFAA(SEQ ID NO: 3)-Ultralink resin to as high as 2.9·10⁹ TU/mL·min of GKEAAFAA(SEQ ID NO: 3)-Poros resin (41-fold higher than the commercial adsorbent CaptureSelect™ Lenti VSVG Affinity Resin.

Finally, all resins except for ToyoPearl®750 and Ultralink afforded more than 100-fold reduction of HCPs. These results demonstrated that the LVV purification performance, while primarily driven by peptide GKEAAFAA (SEQ ID NO: 3), is also determined to a significant extent by the morphological properties of the base matrix. Based on these results, GKEAAFAA (SEQ ID NO: 3)-Poros™ resin, whose binding capacity is ˜2-fold higher than the commercial standard VSV-G Capture Select, was selected for further characterization.

TABLE 11
Properties and performance of chromatography resins functionalized with peptide ligand GKEAAFAA (SEQ ID NO: 3).
The resins were packed in 1 mL column, equilibrated with 100 mM NaCl in 50 mM PIPES buffer at pH 7.4, and loaded with
clarified HEK293 cell culture fluid (LVV titer: 5-9 · 10⁹ TU/mL; HCP titer: 0.05 mg/mL) at the residence time (RT) of
1 min; following washing, LVV elution was conducted using 0.650M NaCl in 50 mM PIPES at pH 7.4 at RT of 1 min.
Productivity was calculated as the number of cell-cell-transducing LVV units purified by 1 mL of resin in 1 min.

| | | | | | | Recovery of | | |
|---|---|---|---|---|---|---|---|---|
| | | | Bead | Pore | DBC_{10%} | cell- | | |
| | | | size | diameter | (TU/mL of | transducing | Productivity | HCP |
| Ligand | Resin | Polymer | (μm) | (nm) | resin) | LVV units | (TU/mL · min) | LRV |
| GKEAAFAA | Poros | PS | 50 | 50-1000 | 4.5 · 10⁹ | 65% | 2.9 · 10⁹ | 2.04 |
| (SEQ ID NO: 3) | ToyoPearl ® | PMMA | 65 | 100 | 4.3 · 10⁹ | 60% | 2.6 · 10⁹ | 2.07 |
| | 650 | | | | | | | |
| | ToyoPearl ® | | 75 | >100 | 5.0 · 10⁹ | 48% | 2.4 · 10⁹ | 1.89 |
| | 750 | | | | | | | |
| | Eshmuno | PVE | 50 | 50 | 2.2 · 10⁹ | 49% | 1.1 · 10⁹ | 2.09 |
| | 500-50 | | | | | | | |
| | Eshmuno | | 50 | 80 | 1.9 · 10⁹ | 61% | 1.2 · 10⁹ | 2.01 |
| | 800-50 | | | | | | | |
| | Genscript | Agarose | 90 | 130 | 5.6 · 10⁸ | 71% | 4.0 · 10⁸ | 2.24 |
| | iodoacetyl | | | | | | | |
| | WorkBead | | 45 | 40 | 3.6 · 10⁸ | 49% | 1.8 · 10⁸ | 2.24 |
| | SulfoLink | | NA | NA | 2.9 · 10⁸ | 56% | 1.6 · 10⁸ | 2.09 |
| | Ultralink | PA/azlactone | 60 | NA | 4.3 · 10⁸ | 55% | 2.4 · 10⁸ | 1.75 |
| Camelid | CaptureSelect ™ | Agarose | 65 | NA | 2.3 · 10⁸ | 58% | 0.7 · 10⁸ | 2.12 |
| Antibody (V_{H}H) | Lenti | | | | | | (RT: 2 min) | |
| | VSVG | | | | | | | |
| | Affinity | | | | | | | |
| | Matrix | | | | | | | |

PS: poly(styrene), PMMA: poly(methyl methacrylate), PVE: poly(vinylether), PAM: polyacrylamide, NA: not available.

### Example 7

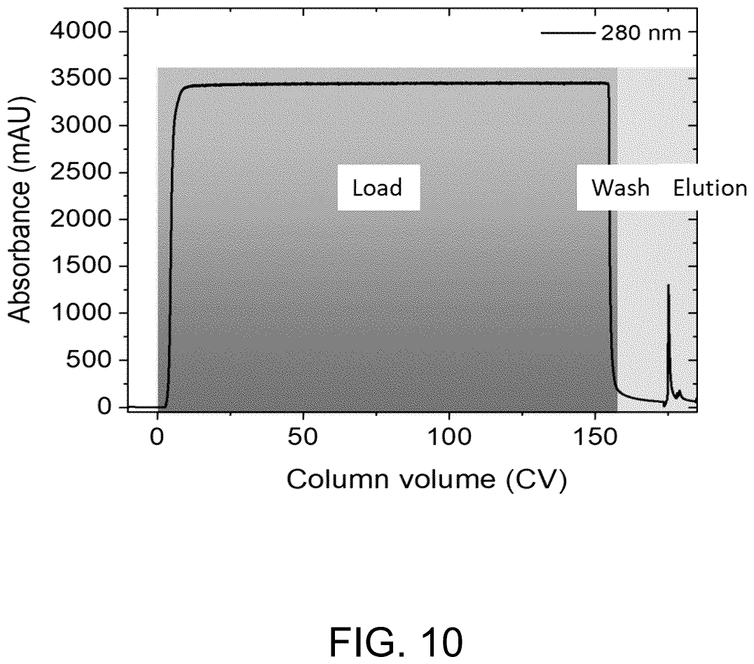

Effect of gene on interest size (GOI) size on LVV purification. Further characterization of GKEAAFAA (SEQ ID NO: 3)-Poros™ resin was conducted by evaluating the effect of GOI sequence and size. Although the size limit of the ssRNA cargo in LVVs is still debated, GOIs up to 10 kbases are considered the limit in terms of process feasibility and sufficient production of transduction units. Therefore, the purification of LVVs packed with a GOI encoding a fusion of CRISPR Cas9 nuclease and GFP (˜9.5 kbases) from a clarified HEK293F cell culture fluid (CCF) was evaluated. As discussed herein, increasing the GOI size leads to lower virus titer, requiring higher loading volume (FIG. 10). The affinity purification afforded 40% virus recovery and a 4.5-fold concentration factor, along with a 65-fold reduction of HCPs to a residual titer of 4.14 g/mL. The lower recovery can be attributed to the loading time, which increased from 30 min for GFP-LVVs to 2.5 hrs for Cas9/GFP-LVVs. Recent studies have shown that longer contact times of LVV with the chromatographic matrix leads to morphologic changes in LVVs and stronger multi-point binding, reducing virus recovery.

### Example 8

Effect of the spacer arm and ligand density on the performance of ligand GKEAAFAA (SEQ ID NO: 3). Introducing a spacer arm between the ligands and the chromatography support has been utilized to promote product capture. Optimizing ligand display is particularly relevant in the context of viral vector purification, owing to the large size and small curvature of the virion surface and the reliance on multi-point interactions to achieve sufficient binding strength and capacity. The interposition of a single G (gly) or a tripeptide GSG (gly-ser-gly) linking the C-terminus of GKEAAFAA (SEQ ID NO: 3) to the POROS™ resin beads was therefore evaluated. The resulting adsorbents were loaded with clarified HEK293 cell culture fluid up to 75% of the anticipated binding capacity (DBC_{10%}˜5·10⁹ TU per mL of resin), at which point no virus was detected in either flow-through or wash fractions. Somewhat unexpectedly, no significant differences in LVV recovery were recorded among the various adsorbents (Table 12).

TABLE 12
Values of recovery of cell-transducing GFP-LVV particles purified from
a clarified HEK293 cell culture fluid (LVV titer: 7·10⁶ TU/mL; HCP
titer: 0.05 mg/mL) using peptide ligand GKEAAFAA (SEQ ID NO: 3)
conjugated to Poros ™ resin using different spacers. The resins were
packed in 1 mL column, equilibrated with 100 mM NaCl in 50 mM PIPES
buffer at pH 7.4, and loaded with 40 mL of fluid at the RT of 1 min;
following washing, LVV elution was conducted using 0.650 M NaCl in
50 mM PIPES at pH 7.4 at RT of 1 min.

| | DBC10% | Recovery of cell- | |
|---|---|---|---|
| | (TU per mL | transducing LVV | HCP |
| Affinity resin | of resin) | units | LRV |
| GKEAAFAA-Poros ™ | 4.5·10⁹ | 65% | 2.04 |
| GKEAAFAA-G-Poros ™ | 1.8·10⁹ | 42% | 2.20 |
| GKEAAFAA-GSG-Poros ™ | 2.1·10⁹ | 62% | 2.02 |
| GKEAAFAA-GSGPGSG-Poros ™ | 2.1·10⁹ | 41% | 2.10 |
| GKEAAFAA-GSGSGSG-Poros ™ | 2.9·10⁹ | 54% | 2.07 |
| GKEAAFAA-PEG3-Poros ™ | 2.8·10⁹ | 28% | 2.08 |

Ligand density can also determine the performance of chromatographic adsorbents; increasing ligand density affords a higher binding capacity but it can also reduce binding selectivity as well as product recovery. To evaluate the impact of ligand density on LVV binding capacity and productivity, four lots of GKEAAFAA(SEQ ID NO: 3)-Poros™ resin ranging from 16 to 69 μmol per mL of resin we produced (Table 13). Binding capacities did not vary substantially with ligand density, increasing of only 1.4-fold against a 4-fold difference in ligand density. Due to the large size of LVV particles, the steric effect is likely the primary factor determining capacity, while ligand density plays only a secondary effect, for an affinity resin of given pore diameter. Conversely, ligand density determines the binding strength due to multi-site interactions (e.g., avidity effect) and therefore product yield during elution. LVV recovery dropped from 80% to 37% as the ligand density increased, causing the productivity to decrease from 5·10⁸ to 3.3·10⁸ cell-transducing unit per mL of resin per minute (no LVV binding was recorded on the control OH-Poros™ resin).

TABLE 13
Values of recovery of cell-transducing GFP-LVV particles purified
from a clarified HEK293 cell culture fluid (LVV titer: 7 ·
10⁶ TU/mL; HCP titer: 0.05 mg/mL) using peptide ligand GKEAAFAA
(SEQ ID NO: 3) conjugated to Poros ™ resin at different
ligand densities. The resins were packed in 1 mL column, equilibrated
with 100 mM NaCl in 50 mM PIPES buffer at pH 7.4, and loaded with
40 mL of fluid at the RT of 1 min; following washing, LVV elution
was conducted using 0.650M NaCl in 50 mM PIPES at pH 7.4 at RT
of 1 min. Productivity was calculated as the number of cell-cell-
transducing LVV units purified by 1 mL of resin in 1 min.

| GKEAAFAA | | | | |
|---|---|---|---|---|
| (SEQ ID NO: 3) | | Recovery of | | |
| ligand density | DBC_{10%} | cell- | | |
| (μmol/mL of | (TU per mL of | transducing | Productivity | HCP |
| resin) | resin) | LVV units | (TU/mL · min) | LRV |
| 69 | 5.3 · 10⁹ | 37% | 3.3 · 10⁸ | 1.94 |
| 34 | 3.9 · 10⁹ | 63% | 4.1 · 10⁸ | 2.04 |
| 28 | 4.4 · 10⁹ | 69% | 5.1 · 10⁸ | 1.93 |
| 16 | 3.7 · 10⁹ | 80% | 5.0 · 10⁸ | 1.99 |

| | | | |
|---|---|---|---|
| Not Function- | <1 · 10⁷ | <1% | — |
| alized (Control) | | | |

### Example 9

Lifetime and stability study of GKEAAFAA-Poros™ resin. The accessibility of gene and cell therapies relies on reducing the cost and increasing the sustainability of their manufacturing. Although most of the production costs are currently associated with upstream materials (e.g., culture media, plasmids, and transfection reagents), the development of stable cell lines for viral vector expression is likely to shift the focus of cost management to the downstream segment. In that context, the lifetime of chromatographic adsorbents is a critical factor in reducing the operating costs and consumables waste stream. Affinity adsorbents in particular, due to their high cost, are expected to be reused over multiple cycles, each followed by a regeneration step using strong denaturing solvents and a cleaning-in-place (CIP) step using caustic conditions. The recommended CIP conditions for most of the affinity resins currently marketed for the purification of AAVs and LVVs are significantly milder (10 mM NaOH) than those routinely applied with established affinity adsorbents like Protein A/G resins (0.5 M NaOH).

The ability of GKEAAFAA (SEQ ID NO: 3)-Poros™ to perform consecutive cycles of LVV purification with intermediate CIP was therefore tested using 0.5 M NaOH (15 CVs in flow followed by 30 min of static contact). The values of binding capacity, yield of encapsidated transgenes and cell-transducing LVV units, and HCP removal measured over 50 cycles, summarized in FIG. 11, clearly demonstrate the stability of GKEAAFAA (SEQ ID NO: 3). The value of LVV DBC_{10%} and yield fluctuated around 2·10⁹ TU per mL of resin and 54%, while the reduction of HEK293 HCP was consistently above 100-fold. These results stem from the amino acid sequence of the peptide ligand, which does not contain residues prone to deamidation, such as Asn (N) or Gln (Q), or oxidation, such as Trp (W), Cys (C), or Met (M).

### Example 10

Membranes as an alternative substrate to chromatography resins for LVV purification. The high values of binding capacity characteristic of chromatography resins are rooted in the large surface area of their pores; however, the tortuous morphology and limited diameter of these pores reduce the transport of large biologics, such as viruses, due to diffusion limitations. The binding of LVV particles was confirmed to be confined to the surface of the beads by loading fluorescently-labeled LVVs on GKEAAFAA (SEQ ID NO: 3)-Poros™ and assessing the distribution of green fluorescence across the bead volume (FIG. 12). Membranes offer an excellent alternative to resins as their open porosity removes diffusive limitations and enable processing significantly higher flow rates at lower pressure drops. To date, however, most of commercial affinity membranes are available for protein purification, whereas only a pseudo-affinity sulfated cellulose membrane is used to purify influenza A with 68% recovery. Developing an affinity membrane is particularly useful for LVV purification, as it would safeguard the transduction activity of the purified virions by reducing their contact time with the chromatographic matrix.

Peptide ligands GKEAAFAA (SEQ ID NO: 3), FEKISNAE (SEQ ID NO: 1), and SRAFVGDADRD (SEQ ID NO: 4) were therefore conjugated on commercial Natrix® membranes and on a commercial activated cellulose membrane. The Natrix® membranes comprise a continuous hydrogel matrix embedded within a polyamide fiber scaffold, the ligands were conjugated on the epoxide groups displayed in the hydrogel matrix and the grafted layer to achieve a functional density (˜0.05 mmol per gram) comparable to that of peptide-functionalized resins. The resulting adsorbents were evaluated by measuring their DBC_{10%} at the residence time (RT) of 0.25 and 0.5 min, along with LVV recovery and HCP clearance (Table 14).

The combination of lower surface area and residence time resulted in a ˜10-fold reduction of binding capacity compared to the lead GKEAAFAA (SEQ ID NO: 3)-Poros™ resin. It is however important to note that GKEAAFAA (SEQ ID NO: 3)-Natrix® and GKEAAFAA (SEQ ID NO: 3)-Cellulose membranes afforded a comparable productivity by capitalizing on the short residence time during all steps of the chromatographic process. The difference in productivity is mainly due to the lower recovery obtained with the affinity membranes (34.0±3.0% and 32±2%, respectively). This can be imputed to the “trapping” of virions in the polymer matrix surrounding or coating the fibers, which reduces the rate of LVV desorption, ultimately translating in lower recovery at short residence time. Notably, the functionalization strategy does not impair the binding selectivity of the affinity membranes, which consistently afforded highly purified LVVs. In particular, SRAFVGDADRD (SEQ ID NO: 4)-Natrix© afforded a remarkable ˜500-fold reduction of HCPs—the highest value reported for LVV affinity purification. In comparison, Mustang Q, a strong anion-exchange membrane widely utilized for LVV capture, afforded a higher product recovery but a significantly lower purity (5-fold lower reduction of HCPs than the affinity membranes).

TABLE 14
Purification performance of affinity membranes functionalized with peptide
ligands GKEAAFAA (SEQ ID NO 3), FEKISNAE (SEQ ID NO: 1), and SRAFVGDADRD (SEQ
ID NO: 4). The membranes were packed in 0.15 mL column, equilibrated with 100 mM NaCl in
50 mM PIPES buffer at pH 7.4, and loaded with clarified HEK293 cell culture fluid (LVV titer:
7·10⁸ TU/mL; HCP titer: 0.05 mg/mL) at the residence time (RT) of either 0.25 or 0.5 min;
following washing, LVV elution was conducted using 0.650 M NaCl in 50 mM PIPES at pH 7.4
at RT of 1 min. Productivity was calculated as the number of cell-cell-transducing LVV
units purified by 1 mL of resin in 1 min.

| | | | Recovery of | | |
|---|---|---|---|---|---|
| | RT | DBC_{10%} | Transducting | Productivity | |
| Membrane | (min) | (TU/mL of resin) | LVVs (%) | (TU/mL·min) | HCP LRV |
| FEKISNAE-Natrix ® | 0.25 | (2.02 + 0.21)·10⁸ | — | | |
| | 0.5 | (3.05 + 0.34)·10⁸ | 29.5 ± 1.5% | 1.8.10⁸ | 1.95 ± 0.11 |
| GKEAAFAA-Natrix ® | 0.25 | (3.15 + 0.21)·10⁸ | — | | |
| | 0.5 | (4.96 + 0.37)·10⁸ | 34.0 ± 3.0% | 3.4·10⁸ | 2.18 ± 0.03 |
| SRAFVGDADRD-Natrix ® | 0.5 | (2.78 + 0.03)·10⁸ | 21.0 ± 0.5% | 1.2·10⁸ | 2.69 ± 0.05 |
| GKEAAFAA-Cellulose | 0.5 | (7.01 + 0.41)·10⁸ | 32.0 ± 2.0% | 4.5·10⁸ | 2.22 ± 0.12 |
| Mustang Q | 0.5 | — | 85.0 ± 0.5% | — | 1.49 ± 0.05 |

### Example 11

Demonstrating a benchtop-scale process for LVV purification. The results obtained in LVV expression and affinity purification coalesced in a X-step downstream process comprising (i) clarification via centrifugation and microfiltration, (ii) affinity-based capture using GKEAAFAA (SEQ ID NO: 3)-Poros™ resin in bind-and-elute mode, (iii) polishing in flow-through mode, (iv) concentration via tangential flow filtration for concentration, and finally (v) diafiltration and sterile filtration. The HEK293 cell culture fluid was initially clarified via centrifugation and filtration using 0.45 μm vacuum filters, and subsequently treated with benzonase to remove residual plasmids and HEK293 host cell DNA (hcDNA). A negligible reduction in the titer of cell-transducing LVV units (˜2%) was observed after filtration.

The clarified fluid was loaded onto GKEAAFAA (SEQ ID NO: 3)-Poros™ resin to 100% of its DBC_{10%}, since prior observations in viral vector purification indicated that slightly overloading the column affords higher values of yield. LVV elution afforded a step LVV yield of 68% and concentration factor of 2.53—corresponding to a productivity of 1.25·10¹⁴ TU·hr⁻¹·Lᵣₑₛᵢₙ⁻¹—together with a 120-fold reduction in HCPs to a residual level of 6.7 g/mL, and residual DNA to an undetectable level. Most notably, the ratio of total vs. infectious particles (TP/IP) decreased significantly at the affinity capture step from 145 to 46, corresponding to a 3-fold enrichment in cell-transducing LVV units (Table 15).

Following the affinity step, a polishing step was implemented to remove residual impurities in flow-through mode using CaptoCore700, which has been utilized in prior studies for the purification of large viral vectors (e.g., lentivirus and adenovirus). A CaptoCore bead comprises an outer layer (shell) whose pores exclude LVVs by size difference while allowing HCPs and other biomolecular contaminants to access the bead's inner, where they are captured by a combination of ion exchange and hydrophobic interactions. The polishing step afforded high yield (95%) and an additional 20-fold reduction in HCPs, achieving a residual level of 0.2 g/mL. The effluent was finally concentrated and buffer-exchanged and sterile-filtered. The process provided a global yield of 33%, delivered a 13,000-fold reduction in HCP content, and undetectable residual DNA by PicoGreen Assay.

TABLE 15
Values of recovery, host cell protein removal, concentration factor e total
particles (TP) to infectious particles (IP) ratio.

| | Step\|Global | Step\|Global | Concentration | |
|---|---|---|---|---|
| Step | Recovery | HCP LRV | factor | TP/IP ratio* |
| Filtration | 98% | N/A | N/A | 145 |
| Affinity Capture | 68%\|67% | 2.07 | 2.53 | 46 |
| (GKEAAFAA-Poros ™) | | | | |
| Polishing | 95%\|64% | 1.18\|3.25 | 0.95 | 59 |
| (CaptoCore 700) | | | | |
| Concentration | 70%\|44% | 0.86\|4.11 | 5.16 | 30 |
| (TFF, MWCO: 100 | | | | |
| kDa) | | | | |
| Sterile filtration | 75%\|33% | N/A | N/A | 41 |
| (0.2 μm filters) | | | | |

*Determined by p24 ELISA and transduction assay.

### Example 12

Optimizing LVV expression in suspension HEK293 cell cultures. Lentiviruses are almost ubiquitously produced by transfecting HEK293F cells using four plasmids: one carrying the gene of interest (GOI), an envelope plasmid, and two packing plasmids. While LVVs can be produced in both adherent or suspended HEK293F cells, production in suspension is preferred owing to its easier scalability, higher titers, and no need of supplementation with FBS. Inspired by studies on the effect of cell culture medium composition on LVV and HCP titers, three cell culture media were tested: Peak Expression, BalanCD HEK293, and LV-Max. The values of LVV titer measured by transduction assay and HCP titer measured by ELISA are collated in FIG. 12. When encapsidating a small GOI encoding for green fluorescent protein (GFP, ˜4.5 kb), no significant difference in LVV titer was observed among the different media; however, the HCP levels in Peak Expression and LV-max media were respectively 1.6- and 2.1-fold higher than those obtained with BalanCD medium (FIG. 12A). This was anticipated, given the role of medium composition in determining cell metabolism and thus HCP production.

The effect of gene payload size on LVV production was also investigated by comparing the GOI encoding for GFP (4.5 kb) with one encoding for GFP-fused CRISPR Cas9 (9.5 kb). The results summarized in FIG. 12B indicate comparable trends, where BalanCD yielded a higher productivity and purity than Peak Expression and LV-Max. However, the functional titer of Cas9-encapsidating LVVs was ˜100-fold lower than their GFP-loaded counterparts. These results match the conclusions of Kumar et al., who investigated the effect of GOI size on LVV titers and observed a similar reduction in functional titer as the transgene size increased. ⁴⁸ The authors presented two hypothesis for titer reduction: (i) larger GOIs lead to a more challenging encapsulation during viral particle assembly and the transport rate of proviral RNA from the cell nucleus to the cytoplasm decreases with its size.

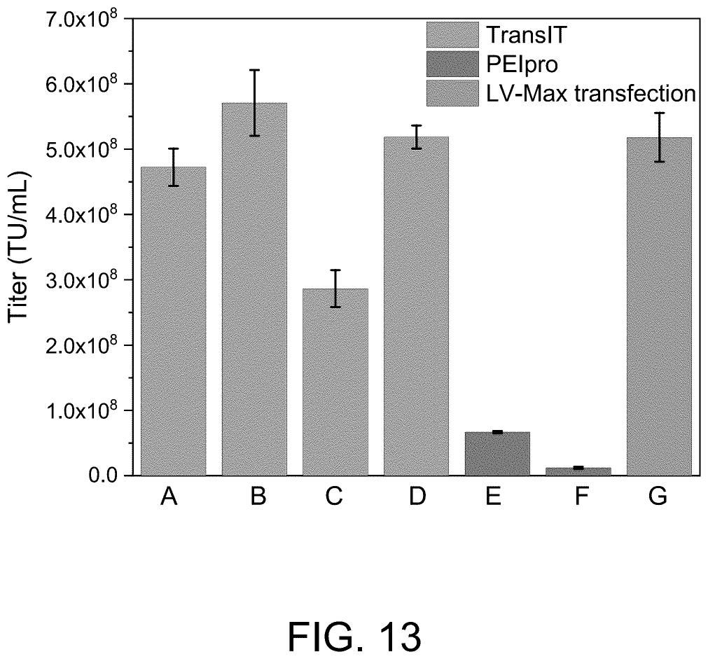

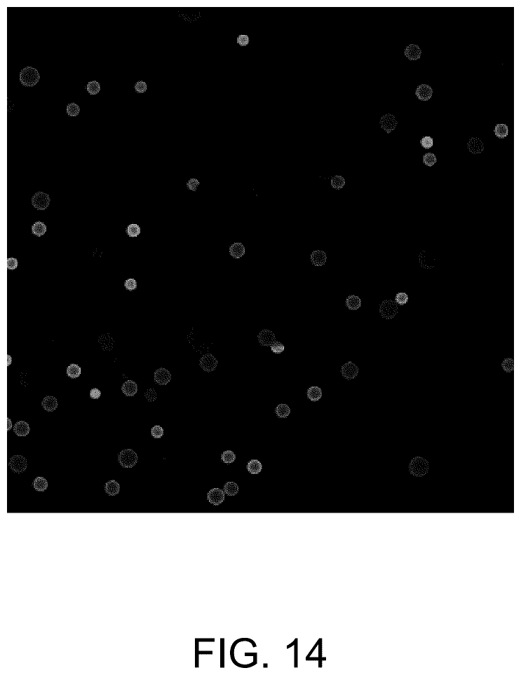

Transient transfection, the commonly used method for viral production, is mediated by a transfection reagent that complexes with the plasmids and shuttles them into the host cells. Different transfection reagents have been described in the literature, including calcium phosphate cationic polymers such as polyethyleneimine (PEI), and lipids. To evaluate the role of transfection reagents on functional titer, three commercial products were tested: TransIT, PEIpro, and LV-Max. The results, summarized in FIG. 13, indicate that TransIT and LV-Max afforded 3-fold higher titers in comparison to PEIpro. Although the exact composition of these transfection reagents is undisclosed, PEIpro is reportedly a pure cationic polymer while TransIT and LV-Max comprise a mixture of cationic polymers and cationic lipids.

Improving the efficiency and cost of downstream processing of viral vectors is critical to make next-generation therapeutics available to all patients in need. While significant advancements have been made in the latest years, especially on the front of AAVs for in vivo gene therapy, a mature purification toolkit for LVV is still missing. Embodiments of the present disclosure include the discovery of selective, robust, and scalable peptide ligands targeting VSV-G pseudotyped LVVs and progressed to the evaluation of bioprocess-relevant parameters—namely, binding capacity, lifetime, productivity, and ability to fit in an end-to-end downstream process. The results of the present disclosure demonstrate the ability of peptide-functionalized adsorbents to (i) capture LVVs carrying different genetic payloads with high binding capacity and (ii) yield eluates enriched in cell-transducing LVV units and depleted in protein and nucleic acid contaminants. The combination of higher purification performance, longer lifetime, and lower cost-of-good compared to commercial affinity adsorbents, position peptide-functionalized resins at the forefront of LVV purification technologies. Additionally, in keeping with the goal of increasing productivity, peptide-functionalized membranes were evaluated that can be operated at a tenth of the resident time characteristic of resin-based chromatography.

### 6. Sequences

Sequences relevant to the embodiments of the present disclosure are provided in the table below.

TABLE 16
Peptide sequences.

| SEQ ID | |
|---|---|
| NO: | Sequence |
| 1 | FEKISNAE |
| 2 | FEKISAAE |
| 3 | GKEAAFAA |
| 4 | SRAFVGDADRD |
| 5 | SFVRIGLSD |
| 6 | SKSAAEHE |
| 7 | GEFENINW |
| 8 | EWKAAFIW |
| 9 | SNEIEIAN |
| 10 | SIEINSSE |
| 11 | FEKISTAE |
| 12 | EHFEHWSE |
| 13 | CGSRQFVADSDRDC |
| 14 | CGSRSFVGDSDRDC |
| 15 | CGSRAFVADADRDC |
| 16 | CGSRAFVGDADC |
| 17 | SRQFVCGDSDRD |
| 18 | SRSFVCDSDRD |
| 19 | AFVGDADRD |
| 20 | NEAIAWSA |
| 21 | SANWAIEW |
| 22 | FFFWKEWE |
| 23 | EKNKEKAN |
| 24 | WFIIEESG |
| 25 | AINNHEWE |
| 26 | ENSNHSAW |
| 27 | FEFSEWAW |
| 28 | NEKWHEAF |
| 29 | IWEFKNHE |
| 30 | WEIAKHSF |
| 31 | LKIWEWEI |
| 32 | SHFENNIW |
| 33 | WFWHAAIF |
| 34 | WLSAAFFH |
| 35 | ESFWFNNE |
| 36 | WHISHAAN |
| 37 | AFWWGHHF |
| 38 | FNNNHEWF |
| 39 | WEIWHFEE |
| 40 | GENNINSN |
| 41 | FIHHIWFS |
| 42 | FWIAIEAI |
| 43 | WESIIFAA |
| 44 | NKIIWANS |
| 45 | NAAFIWNH |
| 46 | NKGHSNEE |
| 47 | SESIIAWW |
| 48 | KAHEHIFW |
| 49 | WSSISEGG |
| 50 | GHFENINW |
| 51 | GASFFISW |
| 52 | GSWGGHHW |
| 53 | HFGNHAHS |
| 54 | SWFHWNGW |
| 55 | GWAANWGF |
| 56 | FGKSAAAA |
| 57 | HENISNSW |
| 58 | NNWHEWHI |
| 59 | AFIHEAWS |
| 60 | GNSEKAAW |
| 61 | HNAWFAAA |
| 62 | FFFAENWE |
| 63 | SNSEWANI |
| 64 | FWSAFINE |
| 65 | NEISSSWF |
| 66 | FSSAAIWN |
| 67 | HAWENNFG |
| 68 | GNSNAAHF |
| 69 | FFFNAFAH |
| 70 | SHIKNSAN |
| 71 | ANFGAHSK |
| 72 | KKWAIGSK |
| 73 | NWEFWSHN |
| 74 | NIFHFNSN |
| 75 | NESHINIS |
| 76 | NWFWSFNE |
| 77 | SKAAAFSH |
| 78 | WIIAWNHE |
| 79 | SHFAWASE |
| 80 | ASWSENNI |
| 81 | IKEIKENN |
| 82 | NNWEAWEN |
| 83 | FNEFNKAN |
| 84 | WKIEENNE |
| 85 | HWEENWAE |
| 86 | NKGHSHEE |
| 87 | CGSRQFVADSDRDC-GSG |
| 88 | CGSRSFVGDSDRDC-GSG |
| 89 | FEKISAAE-GSG |
| 90 | FEKISNAE-GSG |
| 91 | GKEAAFAA-GSG |
| 92 | SRQFVCDSDRD |
| 93 | SRQFVCDSDRD-GSG |
| 94 | SRTFVCDSDRD |
| 95 | SRTFVCDSDRD-GSG |
| 96 | SVFRIGLSD |
| 97 | SVFRIGLSD-GSG |
| 98 | CGSRAFVADADRDC-GSG |
| 99 | CGSRAFVGDADC-GSG |
| 100 | SRQFVCGDSDRD-GSG |
| 101 | SRSFVCDSDRD-GSG |
| 102 | SRAFVGDADRD-GSG |
| 103 | AFVGDADRD-GSG |
| 104 | SFVRIGLSD-GSG |
| 105 | FEKISTAE-GSG |
| 106 | SKSAAEHE-GSG |
| 107 | GKEAAFAA-G |
| 108 | GKEAAFAA-GSGSGSG |
| 109 | GKEAAFAA-GSGPGSG |
| 110 | FEKISNAEC |
| 111 | GKEAAFAAC |
| 112 | SRAFVGDADRDC |

What is claimed is:

1. A peptide for purifying a lentivirus from a sample comprising:
at least one cationic amino acid and/or at least one anionic amino acid;
at least one aromatic amino acid; and
one or more aliphatic amino acids.

2. The peptide of claim 1, wherein the peptide is 5 to 20 amino acids in length.

3. The peptide of claim 1 or claim 2, wherein the peptide is at least 8 amino acids in length.

4. The peptide of any one of claims 1-3, wherein the peptide is 8 amino acids in length.

5. The peptide of claim 1 or claim 2, wherein the peptide is 12-20 amino acids in length.

6. The peptide of any one of claims 1-5, wherein the peptide comprises at least one cationic amino acid and at least one negatively charged amino acid.

7. The peptide of any one of claims 1-6, wherein a cationic amino acid is adjacent to an anionic amino acid.

8. The peptide of any one of claims 1-7, wherein each of the at least one cationic amino acid is individually selected from histidine, lysine, and arginine.

9. The peptide of any one of claims 1-8, wherein the at least one cationic amino acid is lysine.

10. The peptide of any one of claims 1-9, wherein each of the at least one anionic amino acid is individually selected from aspartic acid and glutamic acid.

11. The peptide of any one of claims 1-10, wherein the at least one anionic amino acid is glutamic acid.

12. The peptide of any one of claims 1-11, wherein each of the at least one aromatic amino acid is individually selected from histidine, phenylalanine, tyrosine, and tryptophan.

13. The peptide of any one of claims 1-12, wherein the at least one aromatic amino acid is phenylalanine.

14. The peptide of any one of claims 1-12, wherein the at least one aromatic amino acid is histidine.

15. The peptide of any one of claims 1-14, wherein the one or more aliphatic amino acids are selected from alanine, glycine, isoleucine, leucine, proline, and valine.

16. The peptide of any one of claims 1-15, wherein the peptide does not comprise asparagine, glutamine, and/or tryptophan.

17. The peptide of any one of claims 1-16, wherein the peptide is a cyclic peptide.

18. The peptide of any one of claims 1-17, wherein the cyclic peptide is cyclized via disulfide bond between two cysteine residues.

19. The peptide of any one of claims 1-18, wherein the peptide comprises an amino acid sequence having at least 80% sequence identity with one of SEQ ID NOs: 1-86.

20. The peptide of any one of claims 1-19, wherein the peptide comprises an amino acid sequence having one of SEQ ID NOs: 1-86.

21. The peptide of any one of claims 1-20, wherein the peptide comprises an amino acid sequence having one of SEQ ID NOs: 1, 3, or 4.

22. The peptide of any one of claims 1-18, wherein the peptide comprises an amino acid sequence having 1, 2, 3, 4, or 5 substitutions as compared with one of SEQ ID NOs: 1-86.

23. The peptide of any one of claims 1-20, wherein the peptide comprises an amino acid sequence having 1, 2, 3, 4, or 5 substitutions as compared with one of SEQ ID NOs: 1, 3, and 4.

24. The peptide of any one of claims 1-22, wherein the peptide further comprises a linker.

25. The peptide of claim 24, wherein the linker is bound to the C-terminus of the peptide, and wherein the linker is a glycine-rich linker.

26. A composition for purifying a lentivirus from a sample comprising at least one peptide of any one of claims 1-25.

27. The composition of claim 26, wherein the at least one peptide is bound to a solid support.

28. The composition of claim 27, wherein the solid support comprises a non-porous or porous particle, a membrane, a plastic surface, a fiber or a woven or non-woven fibermat, a hydrogel, a microplate, a monolith, and/or a microfluidic device.

29. The composition of claim 27 or claim 28, wherein the solid support comprises polymethacrylate, polyolefin, polyester, polystyrene, polysaccharide, polyvinyl ether, iron oxide, silica, titania, agarose, and/or zirconia.

30. An adsorbent comprising at least one peptide of any one of claims 1-25 or a composition of any one of claims 26-29.

31. The adsorbent of claim 30, wherein the adsorbent has a binding capacity of at least 10⁸ cell-transducing LVV units per mL of adsorbent (TU/mL).

32. The adsorbent of claim 30 or claim 31, wherein the adsorbent has a binding capacity of at least 10⁹ cell-transducing LVV units per mL of adsorbent (TU/mL).

33. The adsorbent of any of claims 30-32, wherein the adsorbent affords a productivity of at least 10⁸ cell-transducing LVV units per mL of adsorbent per minute of purification time (TU/mL-min).

34. The adsorbent of any of claims 30-33, wherein the adsorbent affords a productivity of at least 10⁹ cell-transducing LVV units per mL of adsorbent per minute of purification time (TU/mL-min).

35. A method of purifying a lentivirus from a sample, the method comprising:
contacting at least one peptide of any one of claims 1-25, the composition of any one of claims 26-29, or the adsorbent of any one of claims 30-34, with a sample comprising the lentivirus, wherein the at least one peptide binds the lentivirus; and
eluting the lentivirus from the at least one peptide.

36. The method of claim 35, wherein the sample is a biological fluid.

37. The method of claim 36, wherein the biological fluid is a cell culture fluid.

38. The method of claim 36 or claim 37, wherein the biological fluid comprises a supernatant and/or a cellular lysate.

39. The method of any one of claims 35-38, wherein the biological fluid is derived from a virus production cell line.

40. The method of any one of claims 35-39, wherein the method further comprises a washing step before eluting the lentivirus.

41. The method of any one of claims 35-40, wherein the method produces at least a 100-fold reduction in host cell proteins.

42. The method of any one of claims 35-41, wherein the method results in at least a 35% yield for cell-transducing LVV units.

43. The method of any one of claims 35-42, wherein the method results in a productivity of at least 10⁸ cell-transducing LVV units per each milliliter of adsorbent used and per minute of purification time.


SEQUENCE LISTING (115 sequences)

SEQ ID NO: 1 - protein, 8 aa, Synthetic construct
FEKISNAE

SEQ ID NO: 2 - protein, 8 aa, Synthetic construct
FEKISAAE

SEQ ID NO: 3 - protein, 8 aa, Synthetic construct
GKEAAFAA

SEQ ID NO: 4 - protein, 11 aa, Synthetic construct
SRAFVGDADRD

SEQ ID NO: 5 - protein, 9 aa, Synthetic construct
SFVRIGLSD

SEQ ID NO: 6 - protein, 8 aa, Synthetic construct
SKSAAEHE

SEQ ID NO: 7 - protein, 8 aa, Synthetic construct
GEFENINW

SEQ ID NO: 8 - protein, 8 aa, Synthetic construct
EWKAAFIW

SEQ ID NO: 9 - protein, 8 aa, Synthetic construct
SNEIEIAN

SEQ ID NO: 10 - protein, 8 aa, Synthetic construct
SIEINSSE

SEQ ID NO: 11 - protein, 8 aa, Synthetic construct
FEKISTAE

SEQ ID NO: 12 - protein, 8 aa, Synthetic construct
EHFEHWSE

SEQ ID NO: 13 - protein, 14 aa, Synthetic construct
CGSRQFVADSDRDC

SEQ ID NO: 14 - protein, 14 aa, Synthetic construct
CGSRSFVGDSDRDC

SEQ ID NO: 15 - protein, 14 aa, Synthetic construct
CGSRAFVADADRDC

SEQ ID NO: 16 - protein, 12 aa, Synthetic construct
CGSRAFVGDADC

SEQ ID NO: 17 - protein, 12 aa, Synthetic construct
SRQFVCGDSDRD

SEQ ID NO: 18 - protein, 11 aa, Synthetic construct
SRSFVCDSDRD

SEQ ID NO: 19 - protein, 9 aa, Synthetic construct
AFVGDADRD

SEQ ID NO: 20 - protein, 8 aa, Synthetic construct
NEAIAWSA

SEQ ID NO: 21 - protein, 8 aa, Synthetic construct
SANWAIEW

SEQ ID NO: 22 - protein, 8 aa, Synthetic construct
FFFWKEWE

SEQ ID NO: 23 - protein, 8 aa, Synthetic construct
EKNKEKAN

SEQ ID NO: 24 - protein, 8 aa, Synthetic construct
WFIIEESG

SEQ ID NO: 25 - protein, 8 aa, Synthetic construct
AINNHEWE

SEQ ID NO: 26 - protein, 8 aa, Synthetic construct
ENSNHSAW

SEQ ID NO: 27 - protein, 8 aa, Synthetic construct
FEFSEWAW

SEQ ID NO: 28 - protein, 8 aa, Synthetic construct
NEKWHEAF

SEQ ID NO: 29 - protein, 8 aa, Synthetic construct
IWEFKNHE

SEQ ID NO: 30 - protein, 8 aa, Synthetic construct
WEIAKHSF

SEQ ID NO: 31 - protein, 8 aa, Synthetic construct
LKIWEWEI

SEQ ID NO: 32 - protein, 8 aa, Synthetic construct
SHFENNIW

SEQ ID NO: 33 - protein, 8 aa, Synthetic construct
WFWHAAIF

SEQ ID NO: 34 - protein, 8 aa, Synthetic construct
WLSAAFFH

SEQ ID NO: 35 - protein, 8 aa, Synthetic construct
ESFWFNNE

SEQ ID NO: 36 - protein, 8 aa, Synthetic construct
WHISHAAN

SEQ ID NO: 37 - protein, 8 aa, Synthetic construct
AFWWGHHF

SEQ ID NO: 38 - protein, 8 aa, Synthetic construct
FNNNHEWF

SEQ ID NO: 39 - protein, 8 aa, Synthetic construct
WEIWHFEE

SEQ ID NO: 40 - protein, 8 aa, Synthetic construct
GENNINSN

SEQ ID NO: 41 - protein, 8 aa, Synthetic construct
FIHHIWFS

SEQ ID NO: 42 - protein, 8 aa, Synthetic construct
FWIAIEAI

SEQ ID NO: 43 - protein, 8 aa, Synthetic construct
WESIIFAA

SEQ ID NO: 44 - protein, 8 aa, Synthetic construct
NKIIWANS

SEQ ID NO: 45 - protein, 8 aa, Synthetic construct
NAAFIWNH

SEQ ID NO: 46 - protein, 8 aa, Synthetic construct
NKGHSNEE

SEQ ID NO: 47 - protein, 8 aa, Synthetic construct
SESIIAWW

SEQ ID NO: 48 - protein, 8 aa, Synthetic construct
KAHEHIFW

SEQ ID NO: 49 - protein, 8 aa, Synthetic construct
WSSISEGG

SEQ ID NO: 50 - protein, 8 aa, Synthetic construct
GHFENINW

SEQ ID NO: 51 - protein, 8 aa, Synthetic construct
GASFFISW

SEQ ID NO: 52 - protein, 8 aa, Synthetic construct
GSWGGHHW

SEQ ID NO: 53 - protein, 8 aa, Synthetic construct
HFGNHAHS

SEQ ID NO: 54 - protein, 8 aa, Synthetic construct
SWFHWNGW

SEQ ID NO: 55 - protein, 8 aa, Synthetic construct
GWAANWGF

SEQ ID NO: 56 - protein, 8 aa, Synthetic construct
FGKSAAAA

SEQ ID NO: 57 - protein, 8 aa, Synthetic construct
HENISNSW

SEQ ID NO: 58 - protein, 8 aa, Synthetic construct
NNWHEWHI

SEQ ID NO: 59 - protein, 8 aa, Synthetic construct
AFIHEAWS

SEQ ID NO: 60 - protein, 8 aa, Synthetic construct
GNSEKAAW

SEQ ID NO: 61 - protein, 8 aa, Synthetic construct
HNAWFAAA

SEQ ID NO: 62 - protein, 8 aa, Synthetic construct
FFFAENWE

SEQ ID NO: 63 - protein, 8 aa, Synthetic construct
SNSEWANI

SEQ ID NO: 64 - protein, 8 aa, Synthetic construct
FWSAFINE

SEQ ID NO: 65 - protein, 8 aa, Synthetic construct
NEISSSWF

SEQ ID NO: 66 - protein, 8 aa, Synthetic construct
FSSAAIWN

SEQ ID NO: 67 - protein, 8 aa, Synthetic construct
HAWENNFG

SEQ ID NO: 68 - protein, 8 aa, Synthetic construct
GNSNAAHF

SEQ ID NO: 69 - protein, 8 aa, Synthetic construct
FFFNAFAH

SEQ ID NO: 70 - protein, 8 aa, Synthetic construct
SHIKNSAN

SEQ ID NO: 71 - protein, 8 aa, Synthetic construct
ANFGAHSK

SEQ ID NO: 72 - protein, 8 aa, Synthetic construct
KKWAIGSK

SEQ ID NO: 73 - protein, 8 aa, Synthetic construct
NWEFWSHN

SEQ ID NO: 74 - protein, 8 aa, Synthetic construct
NIFHFNSN

SEQ ID NO: 75 - protein, 8 aa, Synthetic construct
NESHINIS

SEQ ID NO: 76 - protein, 8 aa, Synthetic construct
NWFWSFNE

SEQ ID NO: 77 - protein, 8 aa, Synthetic construct
SKAAAFSH

SEQ ID NO: 78 - protein, 8 aa, Synthetic construct
WIIAWNHE

SEQ ID NO: 79 - protein, 8 aa, Synthetic construct
SHFAWASE

SEQ ID NO: 80 - protein, 8 aa, Synthetic construct
ASWSENNI

SEQ ID NO: 81 - protein, 8 aa, Synthetic construct
IKEIKENN

SEQ ID NO: 82 - protein, 8 aa, Synthetic construct
NNWEAWEN

SEQ ID NO: 83 - protein, 8 aa, Synthetic construct
FNEFNKAN

SEQ ID NO: 84 - protein, 8 aa, Synthetic construct
WKIEENNE

SEQ ID NO: 85 - protein, 8 aa, Synthetic construct
HWEENWAE

SEQ ID NO: 86 - protein, 8 aa, Synthetic construct
NKGHSHEE

SEQ ID NO: 87 - protein, 17 aa, Synthetic construct
CGSRQFVADSDRDCGSG

SEQ ID NO: 88 - protein, 17 aa, Synthetic construct
CGSRSFVGDSDRDCGSG

SEQ ID NO: 89 - protein, 11 aa, Synthetic construct
FEKISAAEGSG

SEQ ID NO: 90 - protein, 11 aa, Synthetic construct
FEKISNAEGSG

SEQ ID NO: 91 - protein, 11 aa, Synthetic construct
GKEAAFAAGSG

SEQ ID NO: 92 - protein, 11 aa, Synthetic construct
SRQFVCDSDRD

SEQ ID NO: 93 - protein, 14 aa, Synthetic construct
SRQFVCDSDRDGSG

SEQ ID NO: 94 - protein, 11 aa, Synthetic construct
SRTFVCDSDRD

SEQ ID NO: 95 - protein, 14 aa, Synthetic construct
SRTFVCDSDRDGSG

SEQ ID NO: 96 - protein, 9 aa, Synthetic construct
SVFRIGLSD

SEQ ID NO: 97 - protein, 12 aa, Synthetic construct
SVFRIGLSDGSG

SEQ ID NO: 98 - protein, 17 aa, Synthetic construct
CGSRAFVADADRDCGSG

SEQ ID NO: 99 - protein, 15 aa, Synthetic construct
CGSRAFVGDADCGSG

SEQ ID NO: 100 - protein, 15 aa, Synthetic construct
SRQFVCGDSDRDGSG

SEQ ID NO: 101 - protein, 14 aa, Synthetic construct
SRSFVCDSDRDGSG

SEQ ID NO: 102 - protein, 14 aa, Synthetic construct
SRAFVGDADRDGSG

SEQ ID NO: 103 - protein, 12 aa, Synthetic construct
AFVGDADRDGSG

SEQ ID NO: 104 - protein, 12 aa, Synthetic construct
SFVRIGLSDGSG

SEQ ID NO: 105 - protein, 11 aa, Synthetic construct
FEKISTAEGSG

SEQ ID NO: 106 - protein, 11 aa, Synthetic construct
SKSAAEHEGSG

SEQ ID NO: 107 - protein, 9 aa, Synthetic construct
GKEAAFAAG

SEQ ID NO: 108 - protein, 15 aa, Synthetic construct
GKEAAFAAGSGSGSG

SEQ ID NO: 109 - protein, 15 aa, Synthetic construct
GKEAAFAAGSGPGSG

SEQ ID NO: 110 - protein, 9 aa, Synthetic construct
FEKISNAEC

SEQ ID NO: 111 - protein, 9 aa, Synthetic construct
GKEAAFAAC

SEQ ID NO: 112 - protein, 12 aa, Synthetic construct
SRAFVGDADRDC

SEQ ID NO: 113 - DNA, 19 bp, Synthetic construct
  source 1..19: mol_type = other DNA
cccagttccgcccattctc

SEQ ID NO: 114 - DNA, 23 bp, Synthetic construct
  source 1..23: mol_type = other DNA
gcctcggcctctgcataaataaa

SEQ ID NO: 115 - DNA, 17 bp, Synthetic construct
  source 1..17: mol_type = other DNA
atggctgactaattttt